(19) United States
(12) Patent Application Publication
(10) Pub. No.: US 2025/0224675 A1
(43) Pub. Date: Jul. 10, 2025

(54) RESIST COMPOSITION, RESIST PATTERN FORMATION METHOD, AND COMPOUND

(71) Applicant: TOKYO OHKA KOGYO CO., LTD., Kawasaki-shi (JP)

(72) Inventor: Koshi ONISHI, Kawasaki-shi (JP)

(21) Appl. No.: 18/853,546

(22) PCT Filed: Apr. 21, 2023
(86) PCT No.: PCT/JP2023/015874

§ 371 Date: Oct. 2, 2024

(30) Foreign Application Priority Data
Apr. 26, 2022 (JP) 2022-072112

(51) Int. Cl. G03F 7/039; C07C 69/78; C07C 69/86; C07C 69/92; G03F 7/00; G03F 7/038

(52) U.S. Cl. CPC G03F 7/0395; C07C 69/78; C07C 69/86; C07C 69/92; G03F 7/0382; G03F 7/0388; G03F 7/70033

(57) ABSTRACT

A resist composition having favorable sensitivity, roughness characteristics, and etching resistance; a resist pattern formation method; and a novel compound that is useful as a resin component for the resist composition. The resist composition generates an acid upon exposure to light and changes solubility in developing solutions by the action of the acid, and includes a polymer compound having a constituent unit derived from a compound represented by general formula (a0-m0), as a resin component that changes in solubility in developing solutions by the action of an acid. In general formula (a0-m0), W⁰¹ is a polymerizable-group-containing group; W⁰² is an aromatic hydrocarbon group; Ra⁰¹ is a specific acid-dissociable group; Ra⁰² is an iodine atom or a bromine atom; and n is an integer of 1 or greater

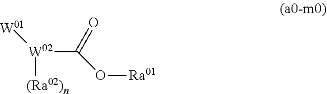

### TECHNICAL FIELD

The present invention relates to a resist composition, a resist pattern formation method, and a compound.

### BACKGROUND ART

In recent years, in the manufacture of semiconductor elements and liquid crystal display elements, advances in lithography technologies have led to rapid progress in the field of pattern miniaturization. These miniaturization methods typically involve shortening the wavelength (increasing the energy) of the exposure light source.

Resist materials require lithography characteristics such as a high resolution that enables reproduction of patterns with minute dimensions, and a high level of sensitivity to these kinds of exposure light sources.

As a resist material that satisfies these requirements, a chemically amplified resist composition containing a base material component whose solubility in a developing solution is changed by an action of an acid and an acid generator component that generates an acid upon light exposure has been used.

In the chemically amplified resist composition, a resin having a plurality of constitutional units is typically used in order to improve lithography characteristics and the like.

For example, Patent Document 1 proposes a resist composition containing a resin component that has a constitutional unit including a specific aromatic carboxylic acid structure substituted with a cyclic acid dissociable group and in which solubility in a developing solution is enhanced.

### CITATION LIST

### Patent Document

### Patent Document 1:

Japanese Unexamined Patent Application, First Publication No. 2019-219469

### SUMMARY OF INVENTION

### Technical Problem

With the further progress of the lithography technology and resist pattern fining, for example, it is aimed to form a fine pattern with a size of several tens of nanometers in lithography by EUV and EB. As a resist pattern dimension decreases, it is required to improve the lithography characteristics such as sensitivity and roughness reduction without the trade-off thereof.

In addition, with further miniaturization of a resist pattern, a resist film will be thinned, and a resist material is required to have further improved etching resistance in a case where etching is performed by using a resist pattern as a mask.

The present invention has been made in view of the above-described circumstances, and an object of the present invention is to provide a resist composition in which sensitivity, roughness characteristics, and etching resistance are all excellent, a resist pattern formation method, and a novel compound useful as a resin component of the resist composition.

### Solution to Problem

In order to achieve the above-described object, the present invention employs the following configurations.

That is, according to a first aspect of the present invention, there is provided a resist composition that generates an acid upon light exposure and in which solubility in a developing solution is changed by an action of the acid, the resist composition including: a resin component (A1) in which solubility in a developing solution is changed by an action of an acid, in which the resin component (A1) has a constitutional unit (a0) derived from a compound represented by General Formula (a0-m0).

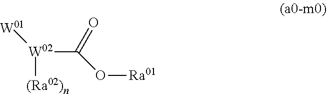

[In the formula, W⁰¹ represents a polymerizable group-containing group. W⁰² represents an aromatic hydrocarbon group which may have a substituent. Ra⁰¹ is an acid dissociable group and represents a group having an alicyclic group, which has no carbon-carbon unsaturated bond, a group having a carbon-carbon unsaturated bond, or a group having an aromatic group. The alicyclic group and the aromatic group may each have a substituent. Ra⁰² represents an iodine atom or a bromine atom, n represents an integer of 1 or greater.]

A second aspect according to the present invention is a resist pattern formation method including: a step of forming a resist film on a support using the resist composition according to the first aspect; a step of exposing the resist film to light; and a step of developing the resist film exposed to light to form a resist pattern.

According to a third aspect of the present invention, there is provided a compound which is represented by General Formula (a0-m0).

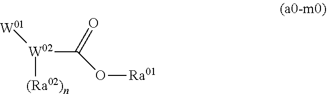

[In the formula, W⁰¹ represents a polymerizable group-containing group. W⁰² represents an aromatic hydrocarbon group which may have a substituent. Ra⁰¹ is an acid dissociable group and represents a group having an alicyclic group, which has no carbon-carbon unsaturated bond, a group having a carbon-carbon unsaturated bond, or a group having an aromatic group. The alicyclic group and the aromatic group may each have a substituent. Ra⁰² represents an iodine atom or a bromine atom, n represents an integer of 1 or greater.]

A fourth aspect according to the present invention is a polymer compound having a constitutional unit derived from the compound represented by General Formula (a0-m0).

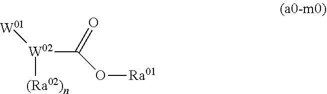

[in the formula, W⁰¹ represents a polymerizable group-containing group. W⁰² represents an aromatic hydrocarbon group which may have a substituent. Ra⁰¹ is an acid dissociable group and represents a group having an alicyclic group, which has no carbon-carbon unsaturated bond, a group having a carbon-carbon unsaturated bond, or a group having an aromatic group. The alicyclic group and the aromatic group may each have a substituent. Ra⁰² represents an iodine atom or a bromine atom, n represents an integer of 1 or greater.]

### Advantageous Effects of Invention

According to the present invention, a resist composition, in which sensitivity, a roughness characteristic, and an etching resistance are all excellent, a resist pattern formation method, and a novel compound useful as a resin component of the resist composition can be provided.

### DESCRIPTION OF EMBODIMENTS

In the present specification and the present claims, the term “aliphatic” is a relative concept used in relation to the term “aromatic”, and defines a group or compound that has no aromaticity.

The term “alkyl group” includes a linear, branched, or cyclic monovalent saturated hydrocarbon group unless otherwise specified. The same applies to the alkyl group in an alkoxy group.

The term “alkylene group” includes a linear, branched, or cyclic divalent saturated hydrocarbon group unless otherwise specified.

Examples of “halogen atom” include a fluorine atom, a chlorine atom, a bromine atom, and an iodine atom.

The term “constitutional unit” indicates a monomer unit constituting a polymer compound (a resin, a polymer, or a copolymer).

The expression “may have a substituent” includes both a case where a hydrogen atom (—H) is substituted with a monovalent group and a case where a methylene (—CH₂—) group is substituted with a divalent group.

The term “light exposure” is a general concept for irradiation with radiation.

The term “base material component” denotes an organic compound having a film-forming ability. Organic compounds used as the base material component are classified into non-polymers and polymers. As the non-polymer, a non-polymer having a molecular weight of 500 or more and less than 4000 (hereinafter, referred to as “low-molecular-weight compound”) is generally used. Hereinafter, “resin”, “polymer compound”, or “polymer” indicates a polymer having a molecular weight of 1000 or greater. As the molecular weight of the polymer, the weight-average molecular weight in terms of polystyrene according to gel permeation chromatography (GPC) is used.

The expression “constitutional unit to be derived” denotes a constitutional unit formed by cleavage of multiple bonds between carbon atoms, for example an ethylenic double bond.

In “acrylic acid ester”, the hydrogen atom bonded to the carbon atom at the α-position may be substituted with a substituent. The substituent (Rαx) that substitutes the hydrogen atom bonded to the carbon atom at the α-position is an atom other than the hydrogen atom or a group. Further, the acrylic acid ester includes itaconic acid diester in which the substituent (R) has been substituted with a substituent having an ester bond and α-hydroxyacryl ester in which the substituent (R) has been substituted with a hydroxyalkyl group or a group obtained by modifying a hydroxyl group thereof. Further, the carbon atom at the α-position of acrylic acid ester indicates the carbon atom to which the carbonyl group of acrylic acid is bonded, unless otherwise specified. Hereinafter, acrylic acid ester in which the hydrogen atom bonded to the carbon atom at the α-position has been substituted with a substituent is also referred to as α-substituted acrylic acid ester.

The term “derivative” includes those obtained by substituting a hydrogen atom at the α-position of a target compound with another substituent such as an alkyl group or a halogenated alkyl group, and derivatives thereof. Examples of the derivatives thereof include a derivative in which the hydrogen atom of the hydroxyl group of the object compound in which the hydrogen atom at the α-position may be substituted with a substituent is substituted with an organic group; and a derivative in which a substituent other than a hydroxyl group is bonded to the object compound in which the hydrogen atom at the α-position may be substituted with a substituent. It is noted that the α-position refers to the first carbon atom adjacent to the functional group unless otherwise specified.

Examples of the substituent that substitutes the hydrogen atom at the α-position of hydroxystyrene include those for R^{αx}.

In the present specification and the scope of the present claims, asymmetric carbons may be present and enantiomers or diastereomers may be present depending on the structures of the chemical formulae. In this case, these isomers are represented by one chemical formula. These isomers may be used alone or in the form of a mixture.

### (Resist Composition)

The resist composition according to the first aspect of the present invention is a resist composition that generates an acid upon light exposure and whose solubility in a developing solution is changed by the action of the acid.

The resist composition according to the embodiment contains a base material component (A) (hereinafter, also referred to as “component (A)”) whose solubility in a developing solution is changed by the action of the acid.

In a case where a resist film is formed using the resist composition according to the present embodiment and the formed resist film is subjected to selective exposure, an acid is generated at exposed portions of the resist film, and the generated acid acts on the component (A) to change the solubility of the component (A) in a developing solution. On the other hand, the solubility of the component (A) in a developing solution is not changed at unexposed portions of the resist film, which generates the difference in solubility in the developing solution between exposed portions and unexposed portions of the resist film.

The resist composition of the present embodiment may be a positive-tone resist composition or a negative-tone resist composition.

Further, in the formation of a resist pattern, the resist composition according to the present embodiment may be applied to an alkali developing process using an alkali developing solution in the developing treatment, or a solvent developing process using an organic developing solution in the developing treatment.

That is, the resist composition according to the present embodiment is a “positive-tone resist composition for an alkali developing process” that forms a positive-tone resist pattern in an alkali developing process, and a “negative-tone resist composition for a solvent developing process” that forms a negative-tone resist pattern in a solvent developing process.

### <Component (A)>

In the resist composition according to the present embodiment, the component (A) contains a resin component (A1) (hereinafter, also referred to as “component (A1)”) whose solubility in a developing solution is changed by the action of an acid, and the component (A1) has a constitutional unit (a0) derived from a compound represented by General Formula (a0-m0).

As the component (A), at least the component (A1) is used, and at least one of other polymer compound and a low-molecular-weight compound may be used in combination with the component (A1).

In the resist composition according to the present embodiment, the component (A) may be used alone or in combination of two or more kinds thereof.

In regard to component (A1)

The component (A1) has a constitutional unit (a0) derived from a compound represented by General Formula (a0-m0).

The component (A1) may have, in addition to the constitutional unit (a), constitutional units other than the constitutional unit (a0).

<<Constitutional Unit (a0)>>

The constitutional unit (a0) is a constitutional unit derived from a compound represented by General Formula (a0-m0).

In such a constitutional unit (a), R^{a01} in Formula (a0-m0) represents an acid dissociable group, and this acid dissociable group protects the oxy group (—O—) side of the carbonyloxy group [—C(═O)—O—] in Formula (a0-m0).

Here, “acid dissociable group” has acid dissociability, which means a bond between the acid dissociable group and an oxygen atom (an oxy group (—O—)) adjacent to the acid dissociable group can be cleaved by the action of an acid. In a case where the acid dissociable group is dissociated by the action of an acid, a polar group (carboxy group) having a polarity higher than that of the acid dissociable group is generated, and thus the polarity is increased. As a result, the polarity of an entire component (A1) is increased. Due to the increase in the polarity, the solubility of the component (A1) in a developing solution is relatively changed such that the solubility is increased in a case where the developing solution is an alkali developing solution and the solubility is decreased in a case where the developing solution is an organic developing solution.

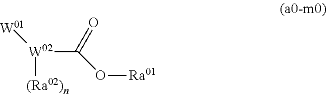

[In the formula, W⁰¹ represents a polymerizable group-containing group. W⁰² represents an aromatic hydrocarbon group which may have a substituent. Ra⁰¹ is an acid dissociable group and represents a group having an alicyclic group, which has no carbon-carbon unsaturated bond, a group having a carbon-carbon unsaturated bond, or a group having an aromatic group. The alicyclic group and the aromatic group may each have a substituent. Ra⁰² represents an iodine atom or a bromine atom, n represents an integer of 1 or greater.]

In Formula (a0-m0), W⁰¹ represents a polymerizable group-containing group.

The term “polymerizable group” as W⁰¹ denotes a group that enables a compound containing a polymerizable group to be polymerized by radical polymerization or the like, which is a group having multiple bonds between carbon atoms, such as an ethylenic double bond.

In the constitutional unit (a0), the multiple bonds in the polymerizable group are cleaved to form a main chain.

Examples of the polymerizable group as W⁰¹ include a vinyl group, an allyl group, an acryloyl group, a methacryloyl group, a fluorovinyl group, a difluorovinyl group, a trifluorovinyl group, a difluorotrifluoromethylvinyl group, a trifluoroallyl group, a perfluoroallyl group, a trifluoromethylacryloyl group, a nonylfluorobutylacryloyl group, a vinyl ether group, a fluorine-containing vinyl ether group, an allyl ether group, a fluorine-containing allyl ether group, a styryl group, a vinylnaphthyl group, a fluorine-containing styryl group, a fluorine-containing vinylnaphthyl group, a norbornyl group, a fluorine-containing norbornyl group, and a silyl group.

The term “polymerizable group-containing group” as W⁰¹ may denote a group formed of only a polymerizable group or a group formed of a polymerizable group and a group other than the polymerizable group. Examples of the group other than the polymerizable group include a divalent hydrocarbon group which may have a substituent and a divalent linking group having a heteroatom.

Divalent Hydrocarbon Group which May have substituent:

In a case where the group other than the polymerizable group represents a divalent hydrocarbon group which may have a substituent, the hydrocarbon group may be an aliphatic hydrocarbon group or an aromatic hydrocarbon group.

Aliphatic Hydrocarbon Group as a Group Other than the polymerizable Group

The aliphatic hydrocarbon group indicates a hydrocarbon group that has no aromaticity. The aliphatic hydrocarbon group may be saturated or unsaturated. In general, it is preferable that the aliphatic hydrocarbon group is saturated.

Examples of the aliphatic hydrocarbon group include a linear or branched aliphatic hydrocarbon group and an aliphatic hydrocarbon group having a ring in the structure thereof.

### Linear or Branched Aliphatic Hydrocarbon Group

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

The above linear or branched aliphatic hydrocarbon group may or may not have a substituent. Examples of the substituent include a fluorine atom, a fluorinated alkyl group having 1 to 5 carbon atoms which has been substituted with a fluorine atom, and a carbonyl group.

### Aliphatic Hydrocarbon Group Having Ring in Structure Thereof

Examples of the aliphatic hydrocarbon group having a ring in the structure thereof include a cyclic aliphatic hydrocarbon group which may have a substituent having a heteroatom in the ring structure thereof (a group in which two hydrogen atoms have been removed from an aliphatic hydrocarbon ring), a group in which the cyclic aliphatic hydrocarbon group is bonded to the terminal of a linear or branched aliphatic hydrocarbon group, and a group in which the cyclic aliphatic hydrocarbon group is interposed in the middle of a linear or branched aliphatic hydrocarbon group. Examples of the linear or branched aliphatic hydrocarbon group include the same groups as described above.

The cyclic aliphatic hydrocarbon group has preferably 3 to 20 carbon atoms and more preferably 3 to 12 carbon atoms.

The cyclic aliphatic hydrocarbon group may be a polycyclic group or a monocyclic group. As the monocyclic alicyclic hydrocarbon group, a group in which two hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group formed by removing two hydrogen atoms from a polycycloalkane is preferable. The number of carbon atoms in the polycycloalkane is preferably 7 to 12, and specifically, adamantane, norbornane, isobornane, tricyclo[5.2.1.0^{2,6}]decane, and tetracyclododecane are exemplary examples.

The cyclic aliphatic hydrocarbon group may or may not have a substituent. Examples of the substituent include an alkyl group, an alkoxy group, a halogen atom, a halogenated alkyl group, a hydroxyl group, and a carbonyl group.

The alkyl group as the substituent is preferably an alkyl group having 1 to 5 carbon atoms, and most preferably a methyl group, an ethyl group, a propyl group, an n-butyl group, or a tert-butyl group.

The alkoxy group as the substituent is preferably an alkoxy group having 1 to 5 carbon atoms, more preferably a methoxy group, an ethoxy group, an n-propoxy group, an iso-propoxy group, an n-butoxy group, or a tert-butoxy group, and most preferably a methoxy group or an ethoxy group.

Examples of the halogen atom for the substituent include a fluorine atom, a chlorine atom, a bromine atom, and an iodine atom, and a fluorine atom is preferable.

Examples of the halogenated alkyl group as the substituent include groups in which some or all hydrogen atoms in the above-described alkyl groups have been substituted with the above-described halogen atoms.

In the cyclic aliphatic hydrocarbon group, some carbon atoms constituting the ring structure thereof may be substituted with a substituent having a heteroatom. As the substituent having a heteroatom, —O—, —C(═O)—O—, —S—, —S(═O)₂—, or —S(═O)₂—O— is preferable.

Aromatic Hydrocarbon Group as Group Other than the Polymerizable Group

The aromatic hydrocarbon group is a hydrocarbon group having at least one aromatic ring.

The aromatic ring is not particularly limited as long as the aromatic ring is a cyclic conjugated system having (4n+2) n electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms. Provided that, the number of carbon atoms in a substituent is not included in the number of carbon atoms. Specific examples of the aromatic ring include aromatic hydrocarbon rings such as benzene, naphthalene, anthracene, and phenanthrene; and aromatic heterocyclic rings in which some carbon atoms constituting the above-described aromatic hydrocarbon rings have been substituted with heteroatoms. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group include a group (an arylene group or a heteroarylene group) obtained by removing two hydrogen atoms from the above-described aromatic hydrocarbon ring or the above-described aromatic heterocyclic ring; a group obtained by removing two hydrogen atoms from an aromatic compound (for example, biphenyl or fluorene) having two or more aromatic rings; and a group (for example, a group obtained by further removing one hydrogen atom from an aryl group in the arylalkyl group such as a benzyl group, a phenethyl group, a 1-naphthylmethyl group, a 2-naphthylmethyl group, a 1-naphthylethyl group, or a 2-naphthylethyl group) obtained by substituting one hydrogen atom of a group (an aryl group or a heteroaryl group) obtained by removing one hydrogen atom from the above aromatic hydrocarbon ring or the above aromatic heterocyclic ring, with an alkylene group. The above-described alkylene group bonded to the aryl group or heteroaryl group has preferably 1 to 4 carbon atoms, more preferably 1 or 2 carbon atoms, and particularly preferably 1 carbon atom.

In the aromatic hydrocarbon group, the hydrogen atom in the aromatic hydrocarbon group may be substituted with a substituent. For example, the hydrogen atom bonded to the aromatic ring in the aromatic hydrocarbon group may be substituted with a substituent. Examples of substituents include an alkyl group, an alkoxy group, a halogen atom, a halogenated alkyl group, and a hydroxyl group.

The alkyl group as the substituent is preferably an alkyl group having 1 to 5 carbon atoms, and most preferably a methyl group, an ethyl group, a propyl group, an n-butyl group, or a tert-butyl group.

Examples of the alkoxy group, the halogen atom, and the halogenated alkyl group as the substituents include those described as the substituent that substitutes a hydrogen atom in the cyclic aliphatic hydrocarbon group.

### Divalent Linking Group Having Heteroatom:

In a case where the group other than the polymerizable group is a divalent linking group having a heteroatom, preferred examples of the linking group include —O—, —C(═O)—O—, —C(═O)—, —O—C(═O)—O—C(═O)—NH—, —NH—, —NH—C(═NH)— (H may be substituted with a substituent such as an alkyl group, an acyl group, or the like), —S—, —S(═O)₂—, —S(═O)₂—O—, and a group represented by General Formula: —Y²¹—O—Y²²—, —Y²¹—, —Y²¹—C(═O)—O—, —C(═O)—O—Y²¹—, —[Y²¹—C(═O)—O]˜]Y²²—, —Y²¹—O—C(═O)—Y²²— or —Y²¹—S(═O)₂—O—Y²²— [in the formulae, Y²¹ and Y²² each independently represent a divalent hydrocarbon group which may have a substituent, 0 represents an oxygen atom, and in” represents an integer of 0 to 3].

In a case where the divalent linking group having a heteroatom is —C(═O)—NH—. —C(═O)—NH—C(═O)—, —NH—, or —NH—C(═NH)—. H may be substituted with a substituent such as an alkyl group or acyl. The substituent (an alkyl group, an acyl group, or the like) has preferably 1 to 10 carbon atoms, more preferably 1 to 8 carbon atoms, and particularly preferably 1 to 5 carbon atoms.

In General Formula —Y²¹—O—Y²²—, —Y²¹—O—, —Y²¹—C(═O)—O—, —C(═O)—O—Y²¹—, —[Y²¹—C(═O)—O]_{m″}—Y²²—, —Y²¹—O—C(═O)—Y==, or —Y²¹—S(═O)₂—O—Y²²—, Y²¹ and Y²² each independently represent a divalent hydrocarbon group which may have a substituent. Examples of the divalent hydrocarbon group include the same groups as those for the divalent hydrocarbon group which may have a substituent, described in the section of the divalent linking group above.

Y²¹ represents preferably a linear aliphatic hydrocarbon group, more preferably a linear alkylene group, still more preferably a linear alkylene group having 1 to 5 carbon atoms, and particularly preferably a methylene group or an ethylene group.

Y²² represents preferably a linear or branched aliphatic hydrocarbon group and more preferably a methylene group, an ethylene group, or an alkylmethylene group. The alkyl group in the alkylmethylene group is preferably a linear alkyl group having 1 to 5 carbon atoms, more preferably a linear alkyl group having 1 to 3 carbon atoms, and most preferably a methyl group.

In the group represented by Formula —[Y²¹—C(═O)—O]_{m″}—Y²²—, m″ represents an integer of 0 to 3, preferably an integer of 0 to 2, more preferably 0 or 1, and particularly preferably 1. That is, a group represented by Formula —Y²¹—C(═O)—O—Y²²— is particularly preferable as the group represented by Formula —[Y²¹—C(═O)—O]ₘ—Y²². Among these, a group represented by Formula —(CH₂)_{n″}—C(═O)—O—(CH₂)_{b′}— is preferable. In the formula, a′ represents an integer of 1 to 10, preferably an integer of 1 to 8, more preferably an integer of 1 to 5, still more preferably 1 or 2, and most preferably 1, b′ represents an integer of 1 to 10, preferably an integer of 1 to 8, more preferably an integer of 1 to 5, still more preferably 1 or 2, and most preferably 1.

It is suitable that W⁰¹ represents, for example, a group represented by Chemical Formula: C(R^{X11})(R^{X12})═C(R^{X13})—Ya^{x0}—.

In the chemical formula, R^{X11}, R^{X12}, and R^{X13} each represent a hydrogen atom, an alkyl group having 1 to 5 carbon atoms, or a halogenated alkyl group having 1 to 5 carbon atoms, and Ya^{x0} represents a single bond or a divalent linking group.

The alkyl group having 1 to 5 carbon atoms as R^{X11}, R^{X12}, and R^{X13} is preferably a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. The halogenated alkyl group having 1 to 5 carbon atoms is a group in which some or all hydrogen atoms in the alkyl group having 1 to 5 carbon atoms have been substituted with halogen atoms. As the halogen atom, a fluorine atom is particularly preferable.

Among these, R^{X11} and R^{X12} each represent preferably a hydrogen atom, an alkyl group having 1 to 5 carbon atoms, or a fluorinated alkyl group having 1 to 5 carbon atoms, and from the viewpoint of industrial availability, more preferably a hydrogen atom or a methyl group, and particularly preferably a hydrogen atom.

In addition, R^{X13} represents preferably a hydrogen atom, an alkyl group having 1 to 5 carbon atoms, or a fluorinated alkyl group having 1 to 5 carbon atoms, and from the viewpoint of industrial availability, more preferably a hydrogen atom or a methyl group, and particularly preferably a hydrogen atom.

The divalent linking group as Ya^{x0} is not particularly limited, and suitable examples thereof include a divalent hydrocarbon group which may have a substituent and a divalent linking group having a heteroatom, each of which has the same definition as that for the divalent hydrocarbon group which may have a substituent and the divalent linking group having a heteroatom, described as groups other than the polymerizable group represented by W⁰¹.

Among the examples, it is preferable that Ya^{x0} represents an ester bond [—C(═O)—O—or —O—C(═O)—], an ether bond (—O—), a linear or branched alkylene group, an aromatic hydrocarbon group, or a combination thereof, or a single bond.

Among these, Ya^{x0} represents more preferably an ester bond [—C(═O)—O— or —O—C(═O)—] or a single bond and still more preferably a single bond.

In Formula (a0-m0), W⁰² represents an aromatic hydrocarbon group which may have a substituent.

Examples of the aromatic hydrocarbon group in W⁰² include a group obtained by removing (n+1) hydrogen atoms from an aromatic ring which may have a substituent. The aromatic ring here is not particularly limited as long as the aromatic ring is a cyclic conjugated system having (4n+2) π electrons. The aromatic ring has preferably 5 to 30 carbon atoms, more preferably 5 to 20 carbon atoms, still more preferably 6 to 15 carbon atoms, and particularly preferably 6 to 12 carbon atoms. Specific examples of the aromatic ring include aromatic hydrocarbon rings such as benzene, naphthalene, anthracene, and phenanthrene; and an aromatic heterocyclic ring obtained by substituting some carbon atoms constituting the above-described aromatic hydrocarbon ring with a heteroatom. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Examples of the aromatic hydrocarbon group as W⁰² also include a group obtained by removing (n+1) hydrogen atoms from an aromatic compound including an aromatic ring (for example, biphenyl and fluorene) which may have two or more substituents.

Among the above, W⁰² is preferably a group in which (n+1) hydrogen atoms have been removed from benzene, naphthalene, anthracene, or biphenyl, more preferably a group in which (n+1) hydrogen atoms have been removed from benzene or naphthalene, and still more preferably a group obtained by removing (n+1) hydrogen atoms from benzene.

The aromatic hydrocarbon group as W⁰² may have a substituent or may not have a substituent. Examples of the substituent include an alkyl group, a fluorine atom, a chlorine atom, a halogenated alkyl group, a hydroxyl group, an alkoxy group, a carbonyl group, a cyano group, an amino group, a nitro group, and an aryl group, and an alkoxy group is preferable.

The alkyl group as the substituent is preferably an alkyl group having 1 to 5 carbon atoms, and most preferably a methyl group, an ethyl group, a propyl group, an n-butyl group, or a tert-butyl group.

Examples of the halogenated alkyl group as the substituent include groups in which some or all hydrogen atoms in the above-described alkyl groups have been substituted with the above-described halogen atoms.

The substituent is preferably a linear or branched alkyl group having 1 to 5 carbon atoms, more preferably a linear or branched alkyl group having 1 to 3 carbon atoms, still more preferably an ethyl group or a methyl group, and particularly preferably a methyl group.

The aromatic hydrocarbon group as W² preferably has no substituent.

In Formula (a0-m0), Ra⁰² represents an iodine atom or a bromine atom, and represents preferably an iodine atom.

In Formula (a0-m0), n is an integer of 1 or greater, and an upper limit of n is determined according to a structure of the aromatic hydrocarbon group in W², n is, for example, an integer of 1 to 3, and is preferably 1 or 2.

In Formula (a0-m0), Ra⁰¹ represents an acid dissociable group.

Ra⁰¹ is an acid dissociable group and represents a group having an alicyclic group, which has no carbon-carbon unsaturated bond, a group having a carbon-carbon unsaturated bond, or a group having an aromatic group. The alicyclic group and the aromatic group may each have a substituent.

The aromatic group may have a fused ring structure of an alicyclic group and an aromatic group.

The alicyclic group which may have a substituent includes an alicyclic hydrocarbon group and a heteroalicyclic group.

The aromatic group which may have a substituent includes an aromatic hydrocarbon group and a heteroaromatic group.

Examples of the heteroatom in the heteroaromatic group and the heteroalicyclic group include an oxygen atom, a sulfur atom, and a nitrogen atom.

Examples of Ra⁰¹ which is an acid dissociable group include those which have been proposed as the acid dissociable groups of the base resin for a chemically amplified resist composition.

Specific examples of the acid dissociable group of the base resin for a chemically amplified resist composition include “acetal type acid dissociable group”, “tertiary alkyl ester type acid dissociable group”, and “secondary alkyl ester type acid dissociable group” described below.

### Acetal Type Acid Dissociable Group:

Examples of the acid dissociable group in Ra⁰¹ include an acid dissociable group (hereinafter, referred to as “acetal type acid dissociable group”) represented by General Formula (a1-r-1).

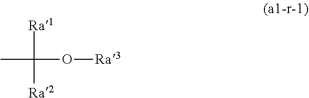

[in the formula, Ra′¹ and Ra′² each represent a hydrogen atom or an alkyl group. Ra′³ represents a group having an alicyclic group, which has no carbon-carbon unsaturated bond or a group having an aromatic group.]

In Formula (a1-r-1), it is preferable that at least one of Ra′¹ or Ra′² represents a hydrogen atom and more preferable that both Ra′¹ and Ra′² represent a hydrogen atom.

In a case where Ra′¹ or Ra′² represents an alkyl group, an alkyl group having 1 to 5 carbon atoms is preferable as the alkyl group. Specific preferred examples thereof include linear or branched alkyl groups. More specific examples thereof include a methyl group, an ethyl group, a propyl group, an isopropyl group, an n-butyl group, an isobutyl group, a tert-butyl group, a pentyl group, an isopentyl group, and a neopentyl group. Among these, a methyl group or an ethyl group is more preferable, and a methyl group is particularly preferable.

The alicyclic group in Ra′³ may be a polycyclic group or a monocyclic group.

The alicyclic group which is a monocyclic group is preferably a group in which one hydrogen atom has been removed from a monocycloalkane. The monocycloalkane has preferably 3 to 6 carbon atoms, and specific examples thereof include cyclopentane and cyclohexane.

As the alicyclic group which is a polycyclic group, a group formed by removing one hydrogen atom from a polycycloalkane is preferable. The number of carbon atoms in the polycycloalkane is preferably 7 to 12, and specifically, adamantane, norbornane, isobornane, tricyclo[5.2.1.0^{2,6}]decane, and tetracyclododecane are exemplary examples.

The aromatic group for Ra′³ is a hydrocarbon group which has at least one aromatic ring. The aromatic ring is not particularly limited as long as the aromatic ring is a cyclic conjugated system having (4n+2) n electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms.

Specific examples of the aromatic ring include aromatic hydrocarbon rings such as benzene, naphthalene, anthracene, and phenanthrene; and aromatic heterocyclic rings in which some carbon atoms constituting the above-described aromatic hydrocarbon rings have been substituted with heteroatoms. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic group as Ra′³ include a group in which one hydrogen atom has been removed from the above-described aromatic hydrocarbon ring or aromatic heterocyclic ring (such as an aryl group or a heteroaryl group); a group in which one hydrogen atom has been removed from an aromatic compound having two or more aromatic rings (such as biphenyl or fluorene); and a group in which one hydrogen atom of the above-described aromatic hydrocarbon ring or aromatic heterocyclic ring has been substituted with an alkylene group (for example, an arylalkyl group such as a benzyl group, a phenethyl group, a 1-naphthylmethyl group, a 2-naphthylmethyl group, a I-naphthylethyl group, or a 2-naphthylethyl group). The number of carbon atoms in the alkylene group bonded to the aromatic hydrocarbon ring or aromatic heterocyclic ring is preferably in a range of 1 to 4, more preferably 1 or 2, and particularly preferably 1.

The alicyclic group and the aromatic group in Ra⁰¹ may each have a substituent.

Examples of the substituent include —R^{P1}, —R^{P2}—O—R^{P1}, —R^{P2}—CO—R^{P1}, —R^{P2}—CO—OR^{P1}, —R^{P2}—O—CO—R^{P1}, —R^{P2}—OH, —R^{N2}—CN, and —R^{P2}—COOH (hereinafter, these substituents will also be collectively referred to as “Ra^{x5}”).

Here, R^{P1} represents a chain-like monovalent saturated hydrocarbon group having 1 to 10 carbon atoms, a monovalent aliphatic cyclic saturated hydrocarbon group having 3 to 20 carbon atoms, or a monovalent aromatic hydrocarbon group having 6 to 30 carbon atoms. Further, R^{P} represents a single bond, a chain-like divalent saturated hydrocarbon group having 1 to 10 carbon atoms, a divalent aliphatic cyclic saturated hydrocarbon group having 3 to 20 carbon atoms, or a divalent aromatic hydrocarbon group having 6 to 30 carbon atoms.

Provided that, some or all hydrogen atoms in the chain-like saturated hydrocarbon group, the aliphatic cyclic saturated hydrocarbon group, and the aromatic hydrocarbon group as R^{P1} and R² may be substituted with fluorine atoms. In the aliphatic cyclic hydrocarbon group, one or more of the above-described substituents may be included as a single kind, or one or more of the above-described substituents may be included as a plurality of kinds.

Examples of the chain-like monovalent saturated hydrocarbon group having 1 to 10 carbon atoms include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2]octanyl group, a tricyclo[5.2.1.0.^{2,6}] decanyl group, a tricyclo[3.3.1.1^{3,7}] decanyl group, a tetracyclo[6.2.1.1^{3,6}.0^{2,7}]dodecanyl group, or an adamantyl group.

Examples of the monovalent aromatic hydrocarbon group having 6 to 30 carbon atoms include a group formed by removing one hydrogen atom from an aromatic hydrocarbon ring such as benzene, biphenyl, fluorene, naphthalene, anthracene, or phenanthrene.

### Tertiary Alkyl Ester Type Acid Dissociable Group:

Examples of the acid dissociable group in Ra⁰¹ include an acid dissociable group represented by General Formula (a1-r-2).

Among examples of the acid dissociable group represented by Formula (a1-r-2), a group formed of an alkyl group is referred to as “tertiary alkyl ester type acid dissociable group”.

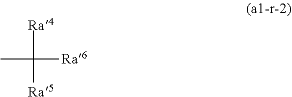

[In the formula. Ra′⁴ to Ra′⁰ each represent a hydrocarbon group, and Ra'⁵ and Ra′⁰ may be bonded to each other to form a ring. Provided that, Ra′⁴ to Ra′⁰ form a group having an alicyclic group, which has no carbon-carbon unsaturated bond, a group having a carbon-carbon unsaturated bond, or a group having an aromatic group.]

Examples of the hydrocarbon group as Ra′⁴ to Ra′⁰ include a linear or branched alkyl group, a chain-like or cyclic alkenyl group, and a cyclic hydrocarbon group.

The linear alkyl group as Ra′⁴ to Ra′⁰ has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group as Ra′⁴ to Ra′⁰ has preferably 3 to 10 carbon atoms and more preferably 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group. Among these, an isopropyl group is preferable.

Examples of the cyclic hydrocarbon group in Ra′⁴ to Ra′⁰ include an aliphatic hydrocarbon group which is a monocyclic group, an aliphatic hydrocarbon group which is a polycyclic group, and an aromatic hydrocarbon group, and the same as those for Ra′ are included.

As the chain-like or cyclic alkenyl group as Ra′⁴ to Ra′⁰, an alkenyl group having 2 to 10 carbon atoms is preferable.

In a case where Ra′⁵ and Ra′⁶ are bonded to each other to form a ring, suitable examples thereof include a group represented by General Formula (a1-r2-1), a group represented by General Formula (a1-r2-2), and a group represented by General Formula (a1-r2-3).

Meanwhile, in a case where Ra′⁴ to Ra′⁰ independently represent a hydrocarbon group without being bonded to one another, suitable examples thereof include a group represented by General Formula (a1-r2-4).

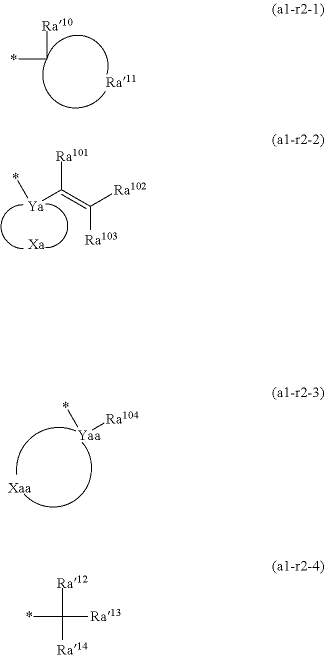

[In Formula (at-r2-1), Ra′¹⁰ represents a linear or branched alkyl group having 1 to 12 carbon atoms, in which a part thereof may be substituted with a halogen atom or a heteroatom-containing group. Ra⁰¹ represents a group forming an aliphatic cyclic group with the carbon atom to which Ra′¹⁰ has been bonded.

In Formula (a1-r2-2), Ya represents a carbon atom. Xa represents a group forming a cyclic hydrocarbon group with Ya. Some or all hydrogen atoms in this cyclic hydrocarbon group may be substituted. Ra¹⁰¹ to Ra¹⁰³ each independently represent a hydrogen atom, a chain-like monovalent saturated hydrocarbon group having 1 to 10 carbon atoms, or a monovalent aliphatic cyclic saturated hydrocarbon group having 3 to 20 carbon atoms. Some or all hydrogen atoms in the chain-like saturated hydrocarbon group and the aliphatic cyclic saturated hydrocarbon group may be substituted. Two or more of Ra¹⁰¹ to Ra¹⁰³ may be bonded to each other to form a cyclic structure.

In Formula (a1-r2-3), Yaa represents a carbon atom. Xaa represents a group forming an aliphatic cyclic group with Yaa. Ra¹⁰⁴ represents an aromatic group which may have a substituent.

In Formula (a1-r2-4), Ra′¹² and Ra′¹³ each independently represent a monovalent hydrocarbon group having 1 to 10 carbon atoms. Some or all hydrogen atoms in this hydrocarbon group may be substituted. Ra′¹⁴ represents a hydrocarbon group which may have a substituent. One or more selected from the group consisting of Ra′¹², Ra′¹³, and Ra′¹⁴ is a group having an alicyclic group, which has no carbon-carbon unsaturated bond, a group having a carbon-carbon unsaturated bond, or a group having an aromatic group.
* represents a bonding site.]

In Formula (a1-r2-1), Ra′¹⁰ represents a linear or branched alkyl group having 1 to 12 carbon atoms, in which a part thereof may be substituted with a halogen atom or a heteroatom-containing group.

The linear alkyl group as Ra′¹⁰ has 1 to 12 carbon atoms, preferably 1 to 10 carbon atoms, and particularly preferably 1 to 5 carbon atoms.

The branched alkyl group as Ra′¹⁰ has preferably 3 to 10 carbon atoms and more preferably 3 to 5 carbon atoms. Specific examples include an isopropyl group, an isobutyl group, a tert-butyl group, an isopentyl group, a neopentyl group, a 1,1-diethylpropyl group, and a 2,2-dimethylbutyl group.

The alkyl group in Ra′¹⁰ may be partially substituted with a halogen atom or a heteroatom-containing group. For example, some hydrogen atoms constituting the alkyl group may be substituted with a halogen atom or a heteroatom-containing group. Further, some carbon atoms (methylene group or the like) constituting the alkyl group may be substituted with a heteroatom-containing group.

Examples of the heteroatoms here include an oxygen atom, a nitrogen atom, and a sulfur atom. Examples of the heteroatom-containing group include (—O—), —C(═O)—O—, —O—C(═O)—, —C(═O)—, —O—C(═O)—O—, —C(═O)—NH—, —NH—, —S—, —S(═O)₂—, and —S(═O)₂—O—.

Ra′¹¹ is preferably the group described as the aliphatic hydrocarbon group (the alicyclic group) which is a monocyclic group or a polycyclic group as Ra′³ in General Formula (a1-r-1). Among them, it is preferably a monocyclic alicyclic hydrocarbon group, and specifically, it is more preferably a cyclopentyl group or a cyclohexyl group. The carbon atoms constituting the alicyclic group may be substituted with a heteroatom or a heteroatom-containing group.

Specific examples of the group represented by Formula (a1-r2-1) are shown below.

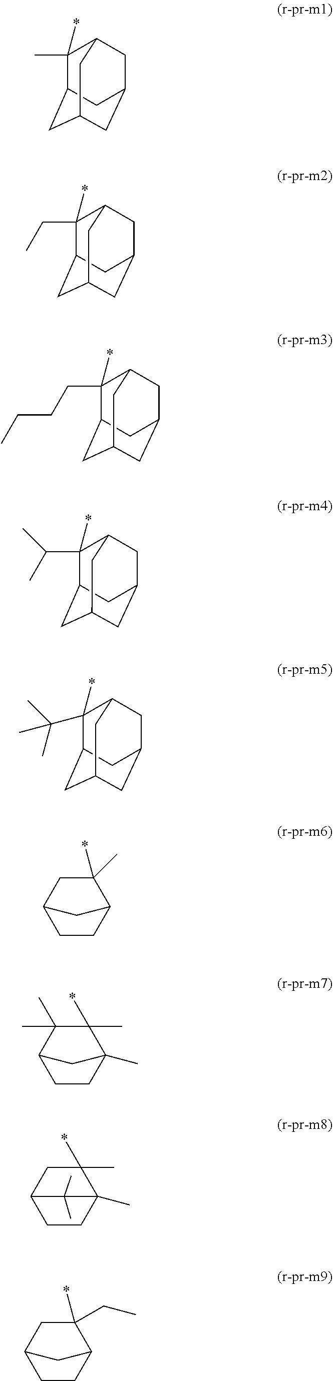

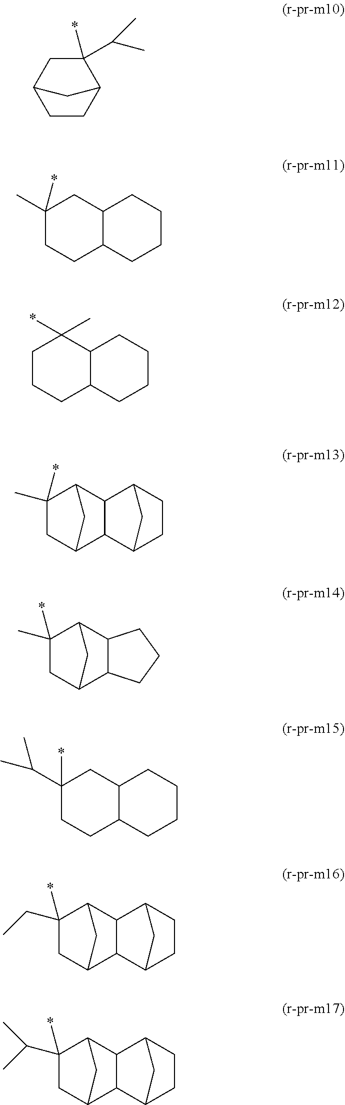

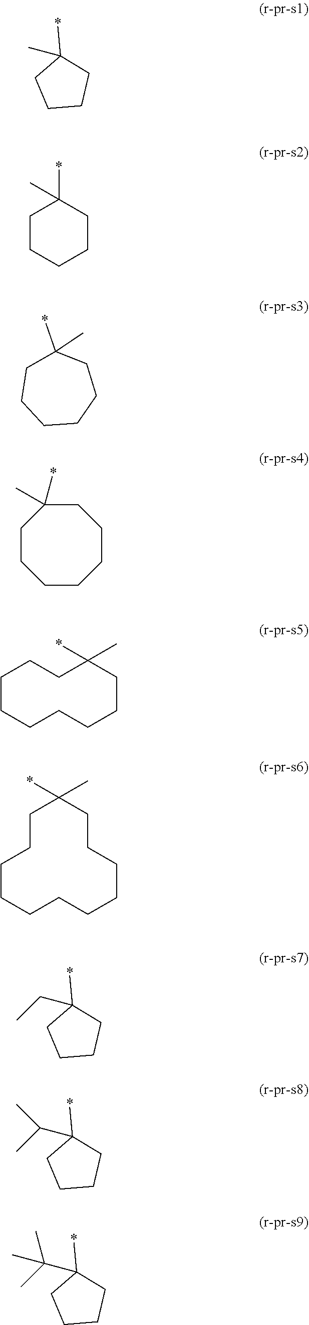

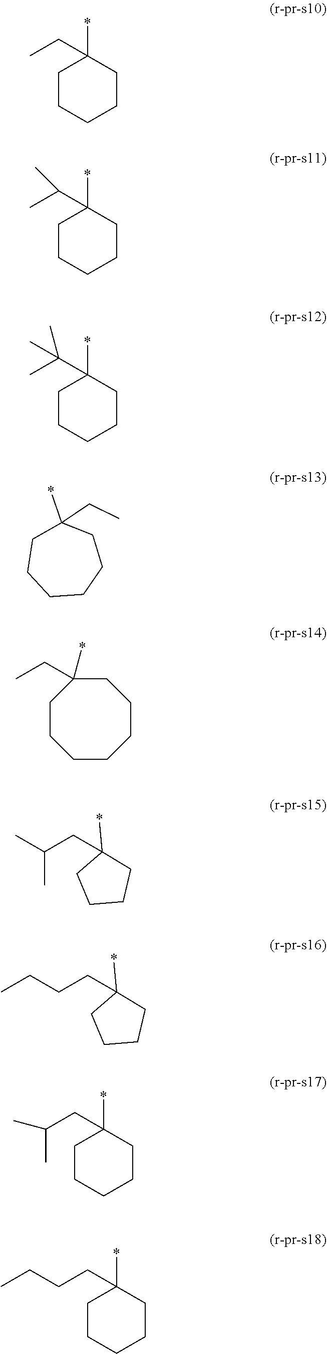

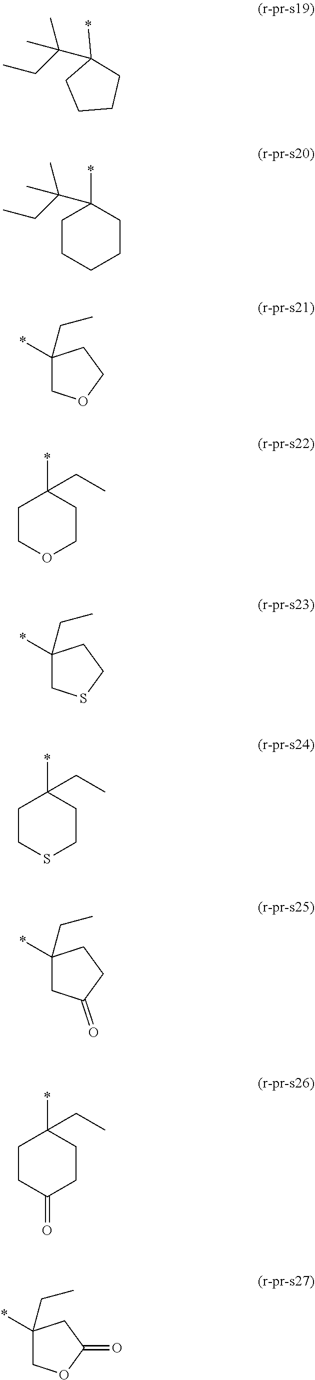

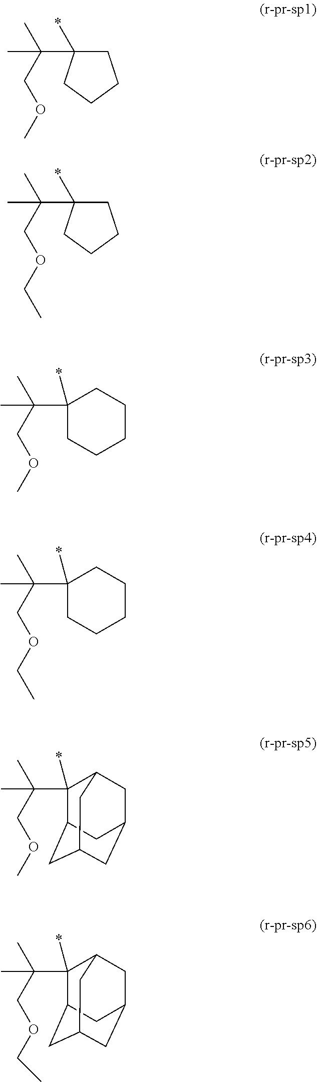

In Formula (a1-r2-2), examples of the cyclic hydrocarbon group that is formed by Xa with Ya include a group in which one or more hydrogen atoms have been further removed from the cyclic monovalent hydrocarbon group (alicyclic group) as Ra′³ in Formula (a1-r-1).

The cyclic hydrocarbon group that is formed by Xa with Ya may have a substituent. Examples of these substituents include the same substituents that the alicyclic group as Ra′³ described above may have. In addition, carbon atoms constituting the cyclic hydrocarbon group formed by Xa and Ya may be substituted with a heteroatom or a heteroatom-containing group.

In Formula (a1-r2-2), examples of the chain-like monovalent saturated hydrocarbon group having 1 to 10 carbon atoms as Ra¹⁰¹ to Ra¹⁰³ include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms as Ra¹⁰¹ to Ra¹⁰³ include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2]octanyl group, a tricyclo[5.2.1.0^{2,6}] decanyl group, a tricyclo[3.3.1.1^{3,7}]decanyl group, a tetracyclo[6.2.1.1^{3,6}0]^{2,7}] dodecanyl group, or an adamantyl group. From the viewpoint of ease of synthesis, Ra¹⁰¹ to Ra¹⁰³ represent preferably a hydrogen atom or a chain-like monovalent saturated hydrocarbon group having 1 to 10 carbon atoms, more preferably a hydrogen atom, a methyl group, or an ethyl group, and particularly preferably a hydrogen atom.

Examples of the substituent included in the chain-like saturated hydrocarbon group or the aliphatic cyclic saturated hydrocarbon group represented by Ra¹⁰¹ to Ra¹⁰³ include the same substituents as those for Ra′^{x5}.

Examples of the group having a carbon-carbon double bond generated by two or more of Ra¹⁰¹ to Ra¹⁰³ being bonded to each other to form a cyclic structure include a cyclopentenyl group, a cyclohexenyl group, a methylcyclopentenyl group, a methylcyclohexenyl group, a cyclopentylidenethenyl group, and a cyclohexylidenethenyl group. Among these, from the viewpoint ofcase of synthesis, a cyclopentenyl group, a cyclohexenyl group, or a cyclopentylidenethenyl group is preferable.

Specific examples of the group represented by Formula (a1-r2-2) are shown below.

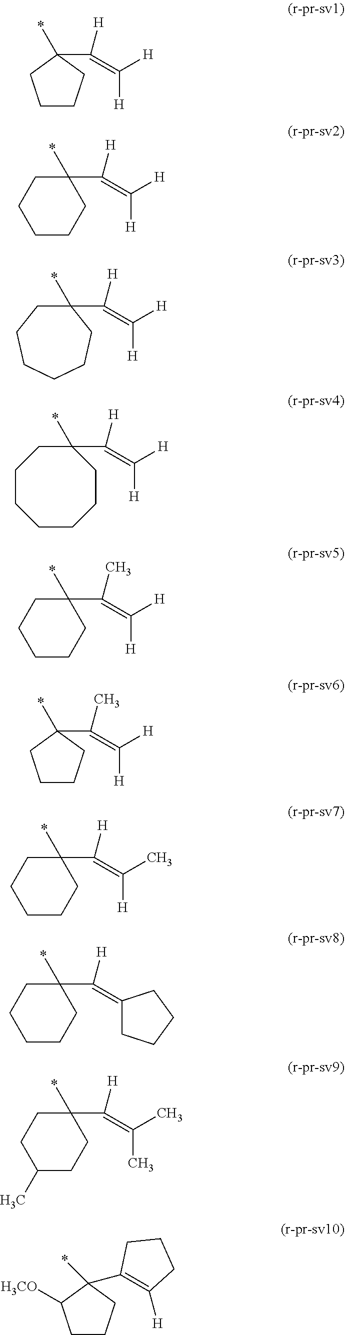

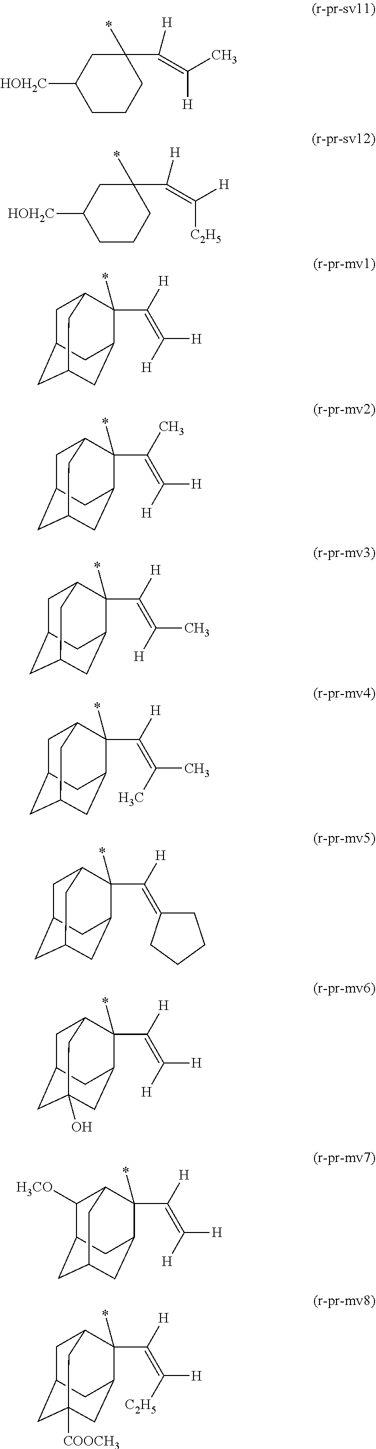

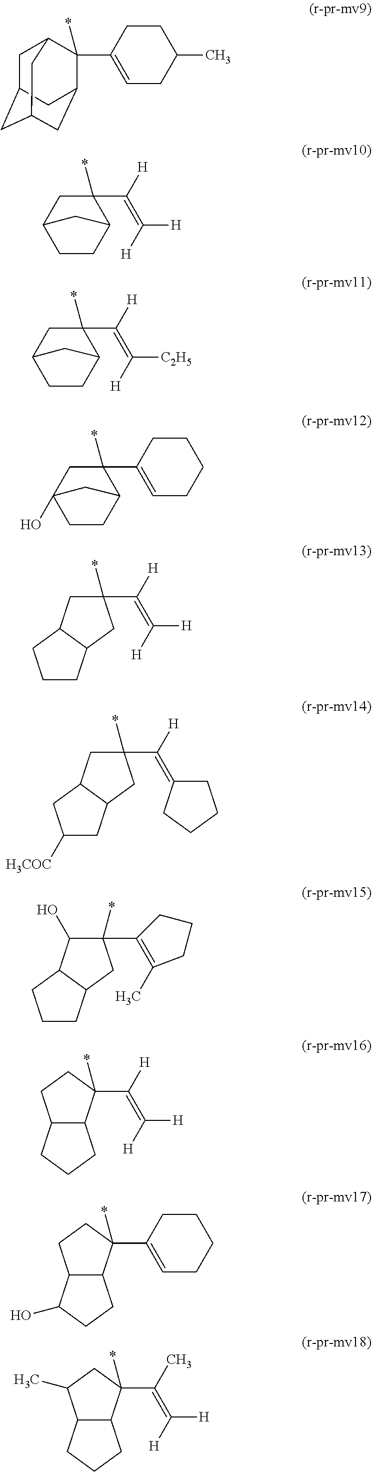

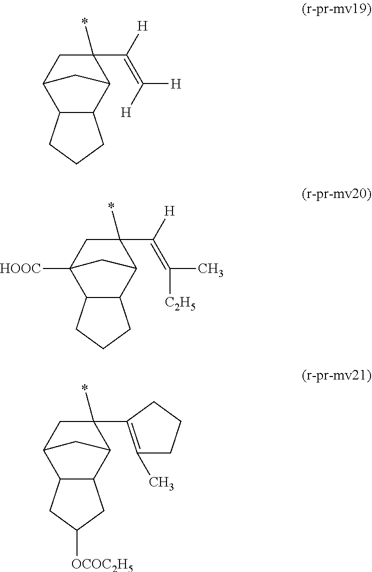

In Formula (a1-r2-3), as the aliphatic cyclic group that is formed by Xaa with Yaa, the group that is an exemplary example of the alicyclic group, which is a monocyclic group or a polycyclic group as Ra′³ in Formula (a1-r-1) is preferable. The aliphatic cyclic group includes an aliphatic cyclic hydrocarbon group and a heteroaliphatic cyclic group.

In Formula (a1-r2-3), examples of the aromatic group as Ra¹⁰⁴ include a group in which one or more hydrogen atoms have been removed from an aromatic ring having 5 to 30 ring-constituting atoms. The aromatic group includes an aromatic hydrocarbon group and a heteroaromatic group. Among the examples. Ra¹⁰⁴ represents preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atom % have been removed from benzene or naphthalene, and most preferably a group in which one or more hydrogen atoms have been removed from benzene.

Examples of the substituent that Ra¹⁰⁴ in Formula (a1-r2-3) may have include a methyl group, an ethyl group, a propyl group, a hydroxyl group, a carboxyl group, a halogen atom, an alkoxy group (a methoxy group, an ethoxy group, a propoxy group, a butoxy group, or the like), and an alkyloxycarbonyl group.

Specific examples of the group represented by Formula (a1-r2-3) are shown below.

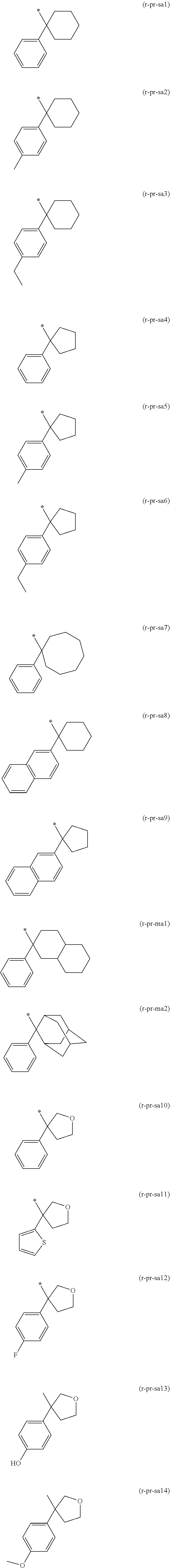

In Formula (a1-r2-4), one or more selected from the group consisting of Ra′¹², Ra′¹³, and Ra′¹⁴ is a group having an alicyclic group, which has no carbon-carbon unsaturated bond, a group having a carbon-carbon unsaturated bond, or a group having an aromatic group.

Examples thereof include a case where Ra⁰¹² and Ra⁰¹ each independently represent a chain-like monovalent hydrocarbon group having 1 to 10 carbon atoms, and Ra′¹⁴ represents a hydrocarbon group (examples of the hydrocarbon group in Ra′¹⁴ include a linear or branched alkyl group, a linear or branched unsaturated hydrocarbon group, or a cyclic hydrocarbon group) which may have a substituent.

Examples of the chain-like monovalent hydrocarbon group having 1 to 10 carbon atoms as Ra′¹² and Ra′¹³ include those which are exemplary examples as the chain-like monovalent hydrocarbon group having 1 to 10 carbon atoms as Ra¹⁰¹ to Ra¹⁰³. Some or all hydrogen atoms in this chain-like hydrocarbon group may be substituted. One or more selected from the group consisting of Ra′¹² and Ra′¹³ may have a carbon-carbon unsaturated bond. Ra′¹² and Ra′¹³ represent preferably an alkyl group having 1 to 5 carbon atoms, more preferably an alkyl group having 1 to 5 carbon atoms, still more preferably a methyl group or an ethyl group, and particularly preferably a methyl group.

In a case where the chain-like hydrocarbon group represented by Ra′¹² and Ra′¹³ is substituted, examples of the substituent thereof include the same substituents as those for Ra^{x5}.

In Formula (a1-r2-4), the linear alkyl group as Ra′¹⁴ has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group as Ra′¹⁴ has preferably 3 to 10 carbon atoms and more preferably 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group. Among these, an isopropyl group is preferable.

Examples of the linear or branched unsaturated hydrocarbon group in Ra′⁴ include an alkenyl group and an alkynyl group.

Examples of the linear alkenyl group in Ra′¹⁴ include a vinyl group, a propenyl group (an allyl group), and a butynyl group. Examples of the branched alkenyl group include a 1-methylvinyl group, a 2-methylvinyl group, a 1-methylpropenyl group, and a 2-methylpropenyl group.

In a case where Ra′¹⁴ represents a cyclic hydrocarbon group, the hydrocarbon group may be an aliphatic hydrocarbon group or an aromatic hydrocarbon group and may be a polycyclic group or a monocyclic group.

As the aliphatic hydrocarbon group which is a monocyclic group, a group in which one hydrogen atom has been removed from a monocycloalkane is preferable. The monocycloalkane has preferably 3 to 6 carbon atoms, and specific examples thereof include cyclopentane and cyclohexane.

As the aliphatic hydrocarbon group which is a polycyclic group, a group formed by removing one hydrogen atom from a polycycloalkane is preferable. The number of carbon atoms in the polycycloalkane is preferably 7 to 12, and specifically, adamantane, norbornane, isobornane, tricyclo[5.2.1.0^{2,6}]decane, and tetracyclododecane are exemplary examples.

Examples of the aromatic hydrocarbon group as Ra′¹⁴ include the same groups as those for the aromatic hydrocarbon group as Ra¹⁰⁴. Among these, Ra′¹⁴ represents preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from naphthalene or anthracene, and most preferably a group in which one or more hydrogen atoms have been removed from naphthalene.

Examples of the substituent that Ra′¹⁴ may have include the same substituents that Ra¹⁰⁴ may have.

In a case where Ra′¹⁴ in Formula (a1-r2-4) represents a naphthyl group, the position bonded to the tertiary carbon atom in Formula (a1-r2-4) may be the 1-position or the 2-position of the naphthyl group.

In a case where Ra′¹⁴ in Formula (a1-r2-4) represents an anthryl group, the position bonded to the tertiary carbon atom in Formula (a1-r2-4) may be the 1-position, the 2-position, or the 9-position of the anthryl group.

Specific examples of the group represented by Formula (a1-r2-4) are shown below.

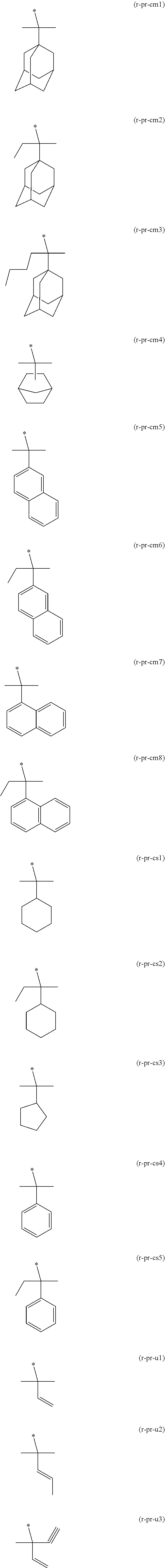

### Secondary Alkyl Ester Type Acid Dissociable Group:

Examples of the acid dissociable group in Ra⁰¹ include an acid dissociable group represented by General Formula (a1-r-4).

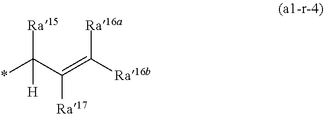

[In the formula, Ra′¹⁵ represents a hydrocarbon group. Ra⁰¹⁶a and Ra′^{b} each independently represent a hydrogen atom, a halogen atom, or an alkyl group. Ra′⁷ represents a hydrogen atom or a hydrocarbon group. Ra′¹⁵ or Ra′¹⁶ and Ra′^{16b} may be bonded to each other to form a ring. Ra′^{16a} or Ra′^{16b} and Ra′¹⁷ may be bonded to each other to form a ring. * represents a bonding site of the carbonyloxy group in General Formula (a0-m0) with an oxygen atom (—O—).

In Formula (a1-r-4), examples of the hydrocarbon group in Ra′¹⁵ and Ra′¹⁷ include a linear or branched alkyl group and a cyclic hydrocarbon group.

The linear alkyl group as Ra⁰¹⁵ has 1 to 12 carbon atoms, preferably 1 to 10 carbon atoms, and particularly preferably 1 to 5 carbon atoms.

The branched alkyl group as Ra′¹⁵ has preferably 3 to 10 carbon atoms and more preferably 3 to 5 carbon atoms. Specific examples include an isopropyl group, an isobutyl group, a tert-butyl group, an isopentyl group, a neopentyl group, a 1, I-diethylpropyl group, and a 2,2-dimethylbutyl group.

Examples of the cyclic hydrocarbon group in Ra′¹⁵ include the same cyclic hydrocarbon group as in Ra′¹⁴ described above.

Examples of the alkyl group as Ra′^{16a} and Ra′^{16b} in Formula (a1-r-4) include the same groups as those for the alkyl group as Ra′¹³.

In Formula (a1-r-4), the hydrocarbon group as Ra′¹⁵ and Ra′¹⁷, and the alkyl group as Ra′^{16a} and Ra′^{16b} may have a substituent. Examples of the substituent include Ra′^{x5} described above.

Ra′¹⁵ or Ra′^{16b} and Ra′^{16b} may be bonded to each other to form a ring.

Ra′^{16a} or Ra′^{16b} and Ra′¹⁷ may be bonded to each other to form a ring.

The rings which may be formed by these being bonded to each other may each be a polycycle or a monocycle, or may be an alicycle or an aromatic ring. The alicycle and the aromatic ring may have a heteroatom.

Among the examples described above, as the ring (ring (x)) formed by Ra′¹⁵ and Ra′^{16a} or Ra′^{16b} being bonded to each other, monocycloalkene, a ring in which some carbon atoms of monocycloalkene have been substituted with heteroatoms (such as an oxygen atom and a sulfur atom), or monocycloalkadiene is preferable, cycloalkene having 3 to 6 carbon atoms is preferable, and cyclopentene or cyclohexene is preferable.

Among the examples described above, as the ring (ring (y)) formed by Ra′^{16a} or Ra′^{16b} and Ra′¹⁷ being bonded to each other, an aromatic ring is preferable, and benzene is particularly preferable. The aromatic ring may have a heteroatom, and examples thereof include a thiophene ring.

Alternatively, the ring (x) and the ring (y) may be bonded to each other to form a condensed ring. Specific examples of the condensed ring include indane.

The ring (x), the ring (y), and the condensed ring formed by the ring (x) and the ring (y) being bonded to each other may each have a substituent. Examples of the substituent include Ra‘ ’ described above.

Specific examples of the group represented by Formula (a1-r-4) are shown below.

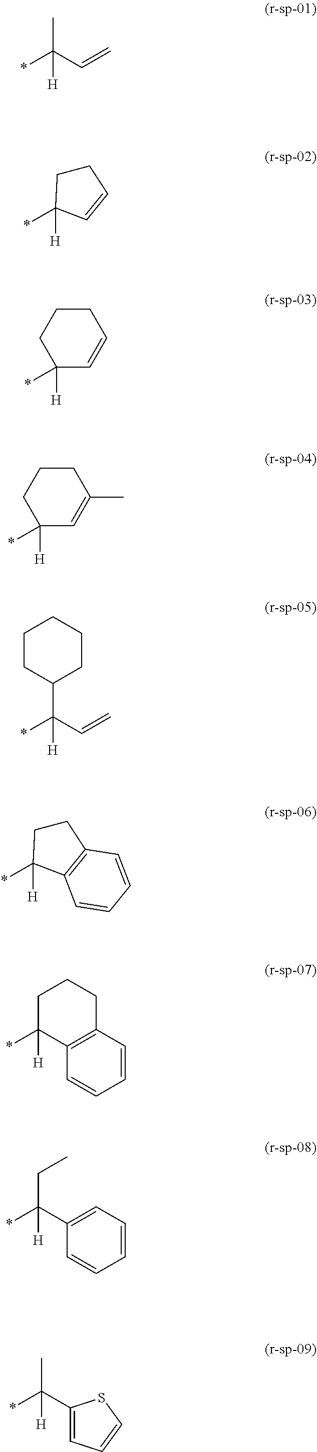

Among the above, Ra⁰¹ represents preferably the “tertiary alkyl ester type acid dissociable group” or the “secondary alkyl ester type acid dissociable group”, and more preferably the acid dissociable group represented by General Formula (a1-r-2) or the acid dissociable group represented by General Formula (a1-r-4).

Among the above, the constitutional unit (a0) is preferably a constitutional unit represented by General Formula (a0-1).

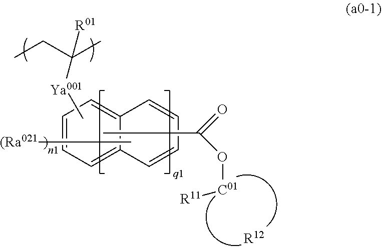

In the formula, R⁰¹ represents an alkyl group having 1 to 5 carbon atoms, a halogenated alkyl group having 1 to 5 carbon atoms, or a hydrogen atom. Ya⁰⁰¹ represents a divalent linking group or a single bond. C⁰¹ represents a tertiary carbon atom. R¹¹ represents a chain-like saturated hydrocarbon group which may have a substituent. R¹² represents a group forming an alicyclic group with C⁰¹, which has no carbon-carbon unsaturated bond. The alicyclic group here may have a substituent. Ra⁰²¹ represents an iodine atom or a bromine atom. q1 represents an integer of 0 to 3, n1 represents an integer of 1 or greater. Provided that, n1≤q1×2+4 is satisfied. The benzene ring in Formula (a0-1) may have a substituent other than Ra⁰²¹.

In Formula (a0-1), R⁰¹ represents an alkyl group having 1 to 5 carbon atoms, a halogenated alkyl group having 1 to 5 carbon atoms, or a hydrogen atom.

As the alkyl group having 1 to 5 carbon atoms as R⁰¹, a linear or branched alkyl group having 1 to 5 carbon atoms is preferable, and specifically, a methyl group, an ethyl group, a propyl group, an isopropyl group, an n-butyl group, an isobutyl group, a tert-butyl group, a pentyl group, an isopentyl group, a neopentyl group, and the like are exemplary examples. The halogenated alkyl group having 1 to 5 carbon atoms is a group in which some or all hydrogen atoms in the alkyl group having 1 to 5 carbon atoms have been substituted with halogen atoms. As the halogen atom, a fluorine atom is particularly preferable.

R⁰¹ represents preferably a hydrogen atom, an alkyl group having 1 to 5 carbon atoms, or a fluorinated alkyl group having 1 to 5 carbon atoms, and it is most preferably a hydrogen atom or a methyl group in terms of industrial availability.

In Formula (a0-1), Ya⁰⁰¹ represents a divalent linking group or a single bond.

The divalent linking group as Ya⁰⁰¹ is not particularly limited, and suitable examples thereof include a divalent hydrocarbon group which may have a substituent and a divalent linking group having a heteroatom, each of which has the same definition as that for the divalent hydrocarbon group which may have a substituent and the divalent linking group having a heteroatom, described as groups other than the polymerizable group represented by W⁰¹.

Among the above, Ya⁰⁰¹ is preferably an ester bond [—C(═O)—O— or —O—C(═O)—], an ether bond (—O—), a linear or branched alkylene group, an aromatic hydrocarbon group, or a combination thereof, or a single bond. Among these, Ya⁰⁰¹ represents more preferably an ester bond [—C(═O)—O— or —O—C(═O)—] or a single bond and still more preferably a single bond.

In Formula (a0-1), the group formed by —C⁰¹(R¹¹)(R¹²) represents an acid dissociable group, and examples thereof include preferably an acid dissociable group represented by General Formula (a1-r2-1), in which R¹² represents a group forming an alicyclic group with C⁰¹, which has no carbon-carbon unsaturated bond.

in Formula (a0-1), Ra⁰²¹ represents an iodine atom or a bromine atom.

In Formula (a0-1), q1 represents an integer of 0 to 3. A case where q1 is 0 gives a benzene structure, a case where q1 is 1 gives a naphthalene structure, a case where q1 is 2 gives an anthracene structure, and a case where q1 is 3 gives a tetracene structure.

In Formula (a0-1), n1 represents an integer of 1 or greater, preferably an integer of 1 to 5, more preferably an integer of 1 to 3, and still more preferably 1 or 2.

Provided that, n1≤q1×2+4 is satisfied.

For example, in a case where q is 0 and has a benzene structure, in the benzene structure, all hydrogen atoms other than the hydrogen atom substituted with (—CH₂—C(R⁰¹)—)—Ya⁰⁰¹— and —C(═O)—O—C⁰¹(R¹¹)(R¹²) group may be substituted with the Ra⁰²¹ group.

In addition, in the benzene structure, the substituent positions of the —C(═O)—O—C⁰¹(R¹¹)(R¹²) group and the Ra⁰²¹ group are each not particularly limited.

Specific examples of the constitutional unit (a0) having a different structure of Ra⁰¹ will be shown below, in which a substituent position with respect to the aromatic hydrocarbon group (W⁰²) is fixed.

In the formulae shown below, R^{α} represents a hydrogen atom, a methyl group, or a trifluoromethyl group. R⁰²⁰ represents an iodine atom or a bromine atom.

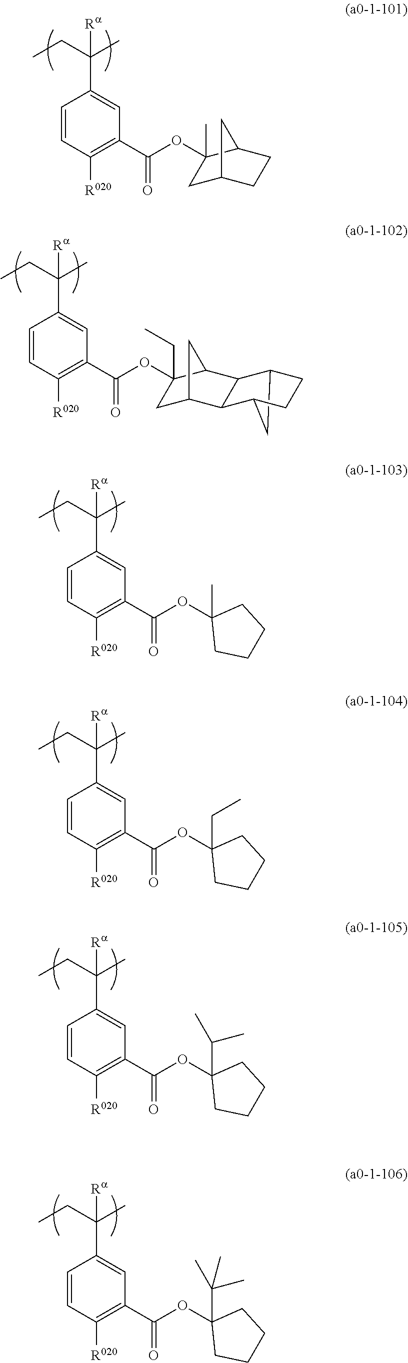

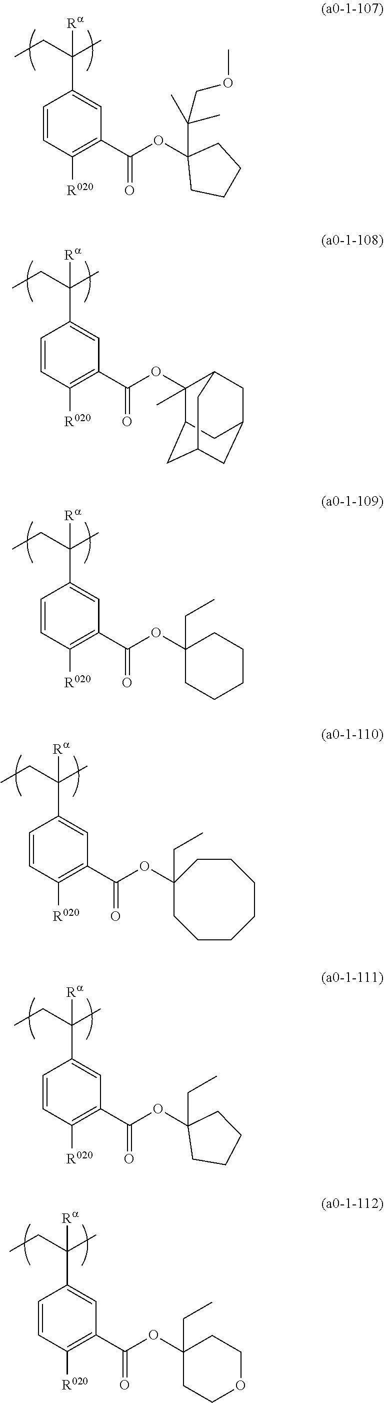

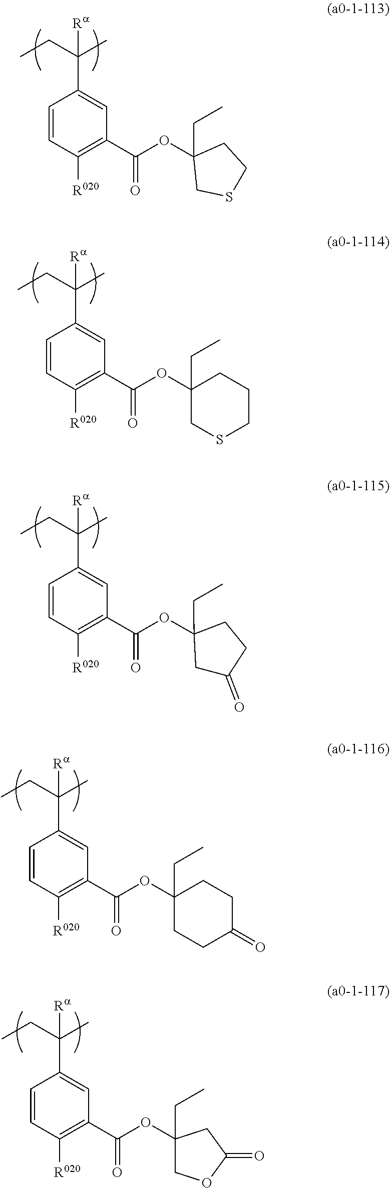

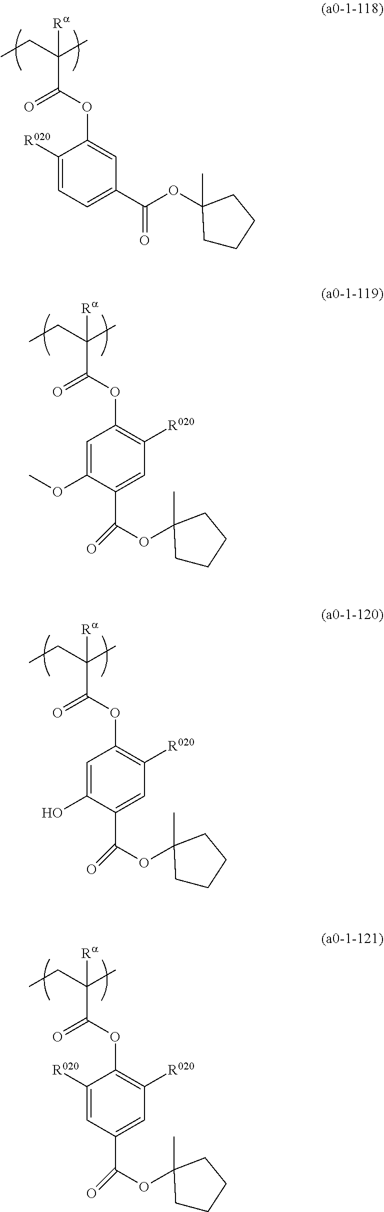

In the resist composition according to the present embodiment, as the constitutional unit represented by General Formula (a0-1), one or more selected from the group consisting of Chemical Formulae (a0-1-101) to (a0-1-121) are preferable, one or more selected from the group consisting of Chemical Formulae (a0-1-101), (a0-1-103), (a0-1-106), (a0-1-108), (a0-1-118), and (a0-1-121) are more preferable, and one or more selected from the group consisting of Chemical Formulae (a0-1-103), (a0-1-106). (a0-1-118), (a0-1-119), and (a0-1-121) are still more preferable, one or more selected from the group consisting of Chemical Formulae (a0-1-119) and (a0-1-121) are particularly preferable,

Chemical Formula (a0-1-121) is most preferable.

Alternatively, the constitutional unit (a0) is preferably the constitutional unit represented by General Formula (a0-2) among the above-described constitutional units.

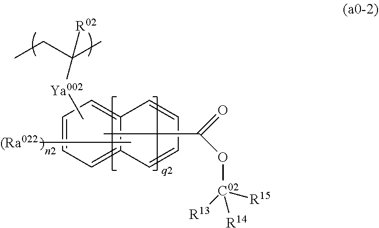

[in the formula, R⁰² represents an alkyl group having 1 to 5 carbon atoms, a halogenated alkyl group having 1 to 5 carbon atoms, or a hydrogen atom. Ya⁰⁰² represents a divalent linking group or a single bond. C⁰² represents a secondary carbon atom or a tertiary carbon atom, and any of α-positions of C⁰² represents a carbon atom constituting a carbon-carbon unsaturated bond with a β-position carbon atom or a carbon atom constituting an aromatic ring. R¹³ represents an aromatic group which may have a substituent, a chain-like unsaturated hydrocarbon group which may have a substituent, or a hydrogen atom. R¹⁴ and R¹⁵ each independently represent a chain-like hydrocarbon group which may have a substituent or a hydrogen atom, or R¹⁴ and R¹⁵ are bonded to each other to form a cyclic group which may have a substituent. Ra⁰²² represents an iodine atom or a bromine atom. q2 represents an integer of 0 to 3, n2 represents an integer of 1 or greater. Provided that, n2≤q2×2+4 is satisfied. The benzene ring in Formula (a0-2) may have a substituent other than Ra⁰²².]

In Formula (a0-2), —C⁰²(R¹³)(R¹⁴)(R¹⁵) group represents an acid dissociable group. C⁰² represents a secondary carbon atom or a tertiary carbon atom.

In Formula (a0-2), in a case where C⁰² represents a secondary carbon atom, any one of R¹³, R¹⁴, or R″ represents a hydrogen atom.

In a case where R¹³ represents a hydrogen atom, R¹⁴ and R″each independently represent a chain-like hydrocarbon group which may have a substituent, or R¹⁴ and R¹⁵ may be bonded to each other to form a cyclic group which may have a substituent. Alternatively, in a case where one of R¹⁴ or R″ represents a hydrogen atom, the other represents a chain-like hydrocarbon group which may have a substituent, and R¹³ represents an aromatic group which may have a substituent or a chain-like unsaturated hydrocarbon group which may have a substituent.

In Formula (a0-2), in a case where C⁰² represents a tertiary carbon atom, R¹, R¹⁴, and R″ are not all hydrogen atoms.

R¹³ represents an aromatic group which may have a substituent or a chain-like unsaturated hydrocarbon group which may have a substituent, and R¹⁴ and R¹⁵ each independently represent a chain-like hydrocarbon group which may have a substituent or R¹⁴ and R″ may be bonded to each other to form a cyclic group which may have a substituent.

In Formula (a0-2), R⁰² represents an alkyl group having 1 to 5 carbon atoms, a halogenated alkyl group having 1 to 5 carbon atoms, or a hydrogen atom, Examples of R⁰² include those described above for R⁰¹.

In Formula (a0-2), Ya⁰⁰² represents a divalent linking group or a single bond. Examples of Ya⁰⁰² include those described above in Ya⁰⁰¹.

In Formula (a0-2), examples of —C⁰²(R¹³)(R¹⁴)(R¹⁵) group include preferably the acid dissociable group represented by General Formula (a1-r2-2), the acid dissociable group represented by General Formula (a1-r2-3), the acid dissociable group represented by General Formula (a1-r2-4), or the acid dissociable group represented by General Formula (a1-r-4).

in Formula (a0-2), Ra⁰⁰² represents an iodine atom or a bromine atom.

In Formula (a0-2), q represents an integer of 0 to 3. A case where q is 0 gives a benzene structure, a case where q is 1 gives a naphthalene structure, a case where q is 2 gives an anthracene structure, and a case where q is 3 gives a tetracene structure.

In Formula (a0-2), n1 represents an integer of 1 or greater, preferably an integer of 1 to 5, more preferably an integer of 1 to 3, and still more preferably 1 or 2.

Provided that, n1≤q1×2+4 is satisfied.

For example, in a case where q is 0 and has a benzene structure, in the benzene structure, all hydrogen atoms other than the hydrogen atom substituted with (—CH₂—C(R⁰²)—)—Ya⁰⁰¹— and —C(═O)—O—C⁰²(R¹³)(R¹⁴)(R¹⁵) group may be substituted with the Ra⁰²² group.

In addition, in the benzene structure, the substituent positions of the —C(═O)—O—C⁰²(R¹³)(¹⁴)R¹⁵) group and the Ra⁰²² group are each not particularly limited.

Specific examples of the constitutional unit (a0) having a different structure of Ra⁰¹ will be shown below, in which a substituent position with respect to the aromatic hydrocarbon group (W⁰²) is fixed.

In the formulae shown below, R^{α} represents a hydrogen atom, a methyl group, or a trifluoromethyl group. R⁰²⁰ represents an iodine atom or a bromine atom.

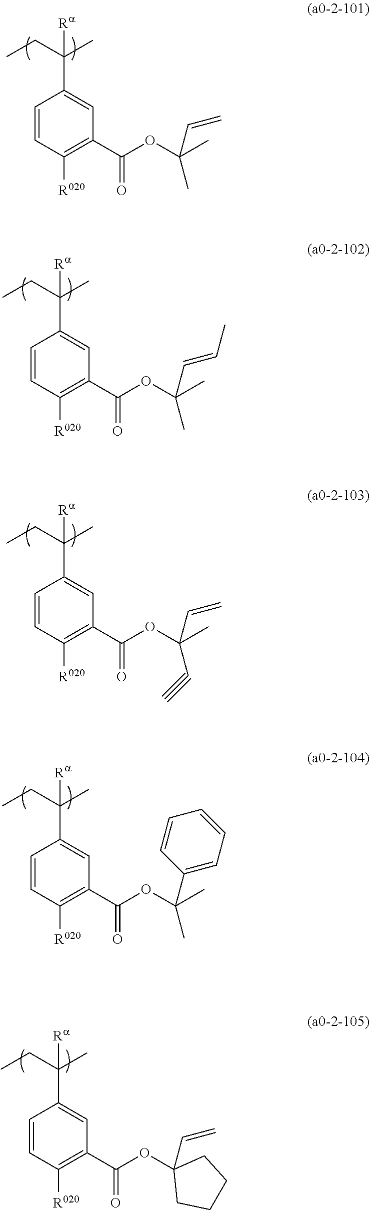

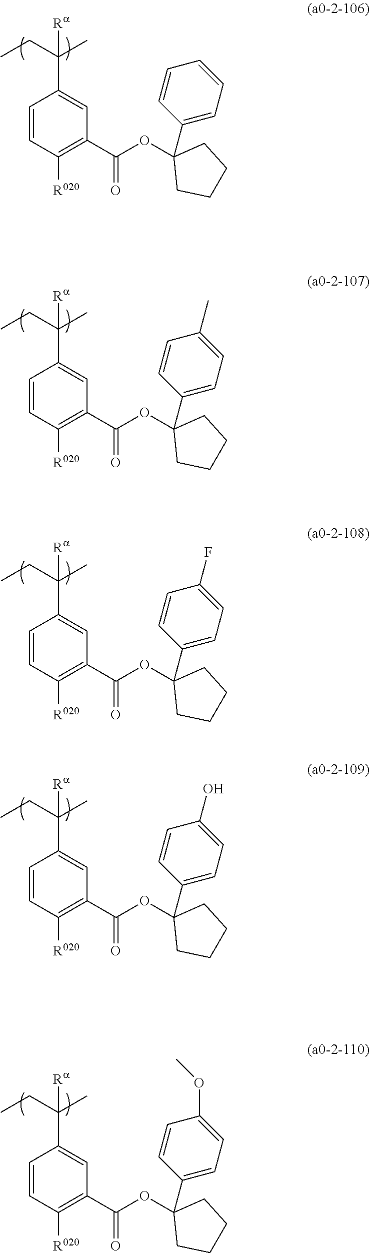

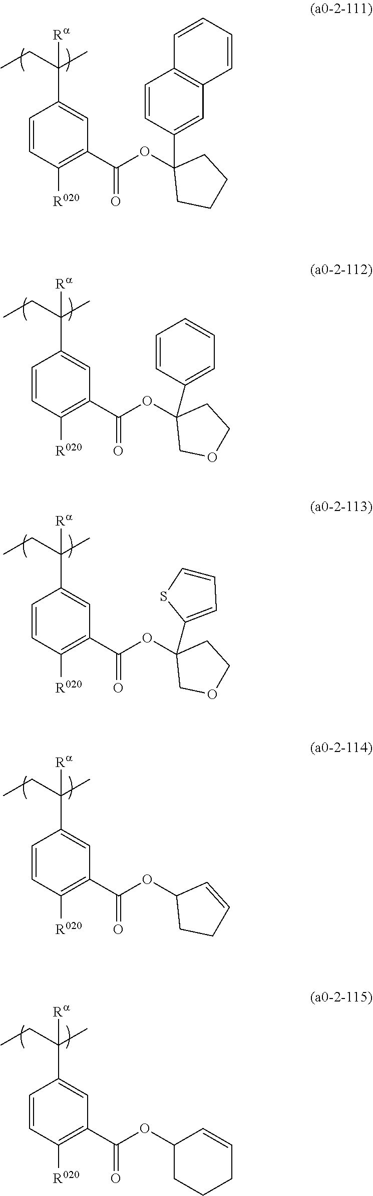

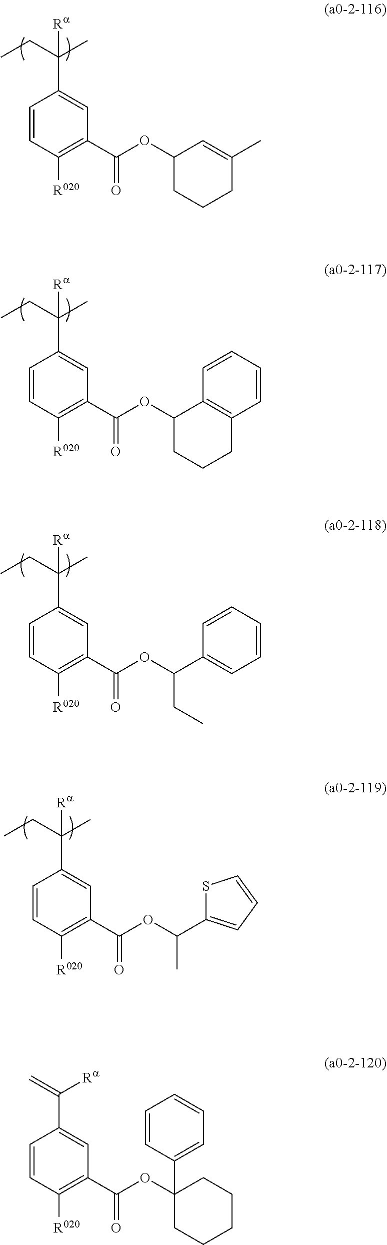

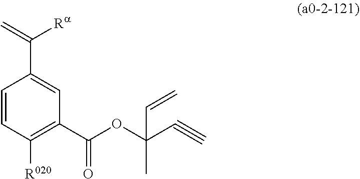

In the resist composition according to the present embodiment, as the constitutional unit represented by General Formula (a0-2), one or more selected from the group consisting of the above-described Chemical Formulae (a0-2-101) to (a0-2-121) are preferable,
one or more selected from the group consisting of Chemical Formulae (a0-2-105), (a0-2-113), (a0-2-115), (a0-2-117), (a0-2-120), and (a0-1-121) are more preferable, and
one or more selected from the group consisting of Chemical Formulae (a0-2-105), (a0-2-113), (a0-2-115), (a0-2-117), and (a0-2-120) are more preferable.

Specific examples of the substituent positions of the —C(═O)—O—Ra⁰¹ group and the Ra⁰² group in the aromatic hydrocarbon group (W⁰²) in General Formula (a0-m0) are shown below.

In the formulae shown below, R″ represents a hydrogen atom, a methyl group, or a trifluoromethyl group. R⁰¹ represents an optional acid dissociable group. R⁰²⁰ represents an iodine atom or a bromine atom.

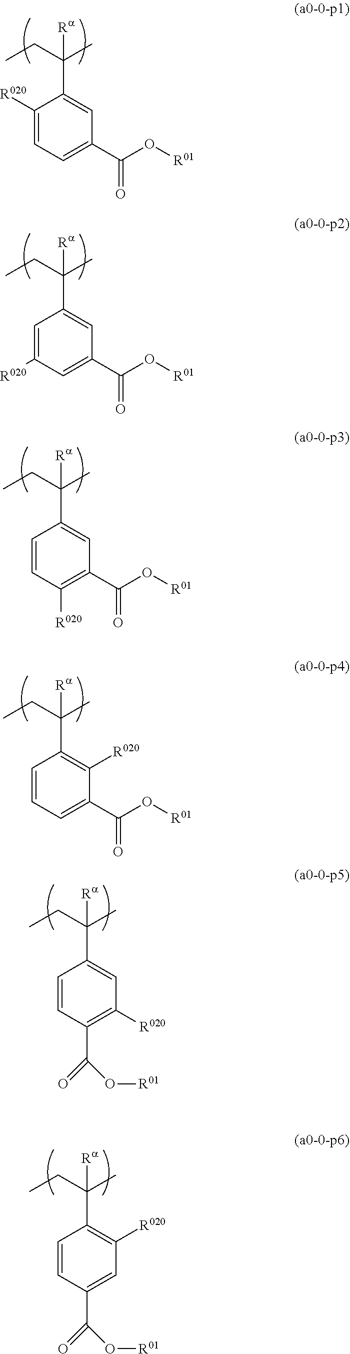

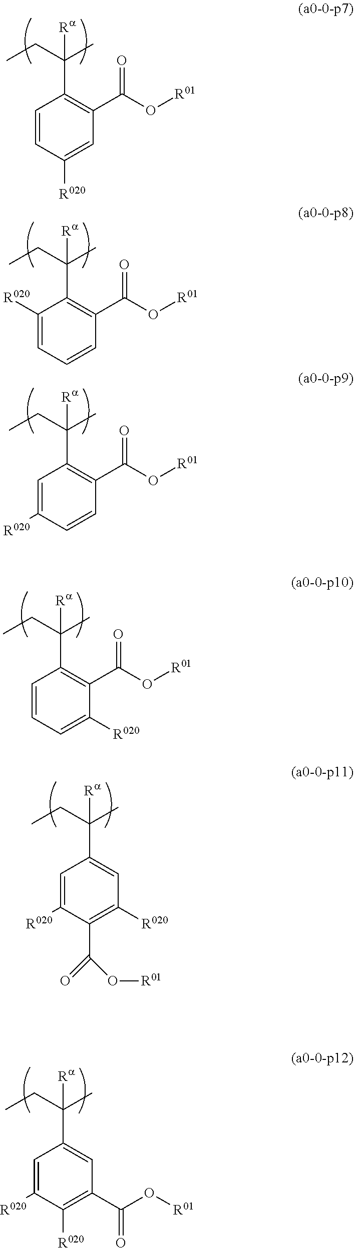

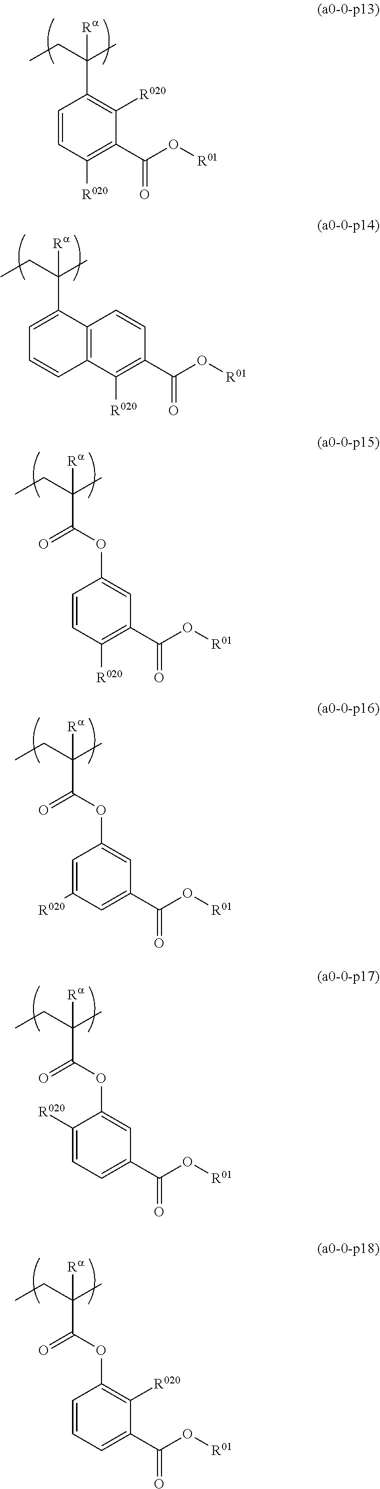

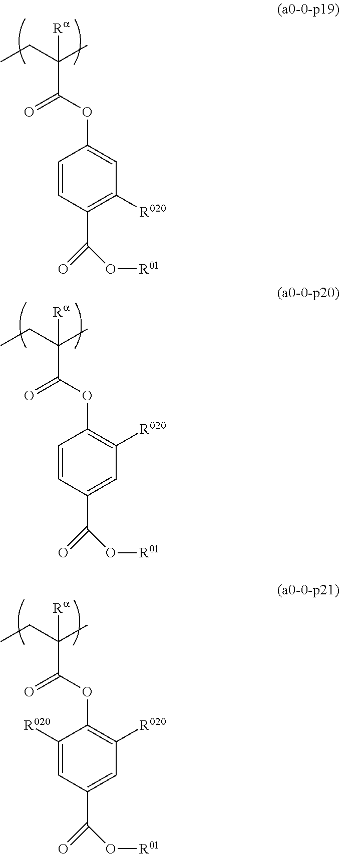

The aromatic hydrocarbon group (W⁰²) in General Formula (a0-m0) may have a substituent R⁰³, in addition to the —C(═O)—O—Ra⁰¹ group and the Ra⁰² group. In the formulae shown below, R″ represents a hydrogen atom, a methyl group, or a trifluoromethyl group. R⁰¹ represents an optional acid dissociable group. R⁰²⁰ represents an iodine atom or a bromine atom. R⁰³ may be an alkyl group, a fluorine atom, a chlorine atom, a halogenated alkyl group, a hydroxyl group, an alkoxy group, a carbonyl group, a cyano group, an amino group, a nitro group, an aryl group, or the like.

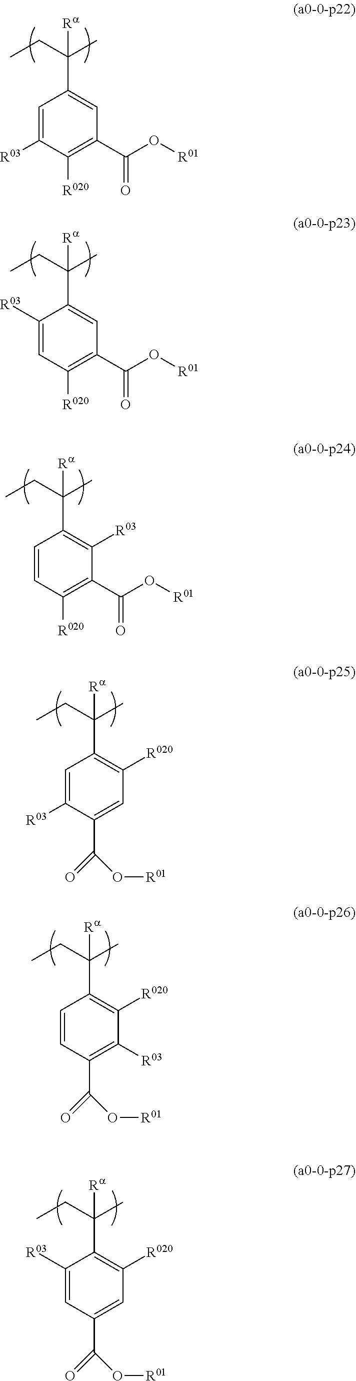

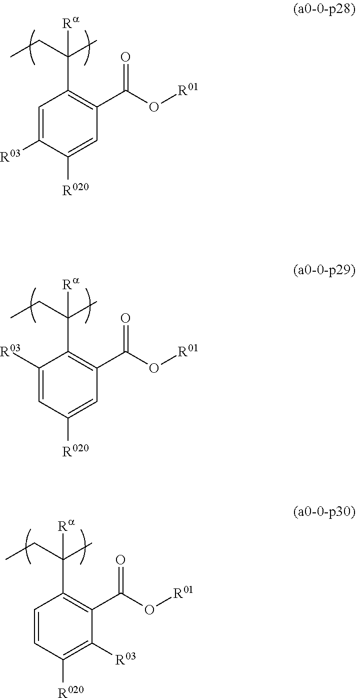

In the resist composition according to the present embodiment, as the constitutional unit (a0), one or more selected from the group consisting of Chemical Formulae (a0-0-p1) to (a0-0-p30) are preferable, and one or more selected from the group consisting of Chemical Formulae (a0-0-p3), (a0-0-p6), (a0-0-p7), (a0-0-p17), (a0-0-p21), and (a0-0-p25) are more preferable.

The constitutional unit (a0) of the component (A1) may be used alone or in combination of two or more kinds thereof.

The proportion of the constitutional unit (a0) in the component (A1) is preferably 20% by mole or greater and 80% by mole or less, more preferably 30% by mole or greater and 70% by mole or less, and still more preferably 40% by mole or greater and 60% by mole or less with respect to the total amount (100% by mole) of all constitutional units constituting the component (A1).

In a case where the proportion of the constitutional unit (a0) is greater than or equal to the lower limits of the above-described preferable ranges, the sensitivity, all the roughness characteristic, and the etching resistance are likely to be enhanced. Meanwhile, in a case where the proportion of the constitutional unit (a0) is less than or equal to the upper limits of the above-described preferable ranges, the constitutional unit (a0) and other constitutional units are likely to be balanced.

<<Other Constitutional Units in Addition to Constitutional Unit (a0)>>

The component (A1) may have other constitutional units as necessary in addition to the constitutional unit (a0) described above.

Examples of other constitutional units include a constitutional unit (a1) containing an acid decomposable group whose polarity is increased by the action of an acid; a constitutional unit (a10) represented by General Formula (a10-1); a constitutional unit (a2) which contains a lactone-containing cyclic group; and a constitutional unit (a8) derived from a compound represented by General Formula (a8-1); a constitutional unit that generates an acid upon light exposure. Further, constitutional units corresponding to the constitutional unit (a0) described above are excluded from the other constitutional units.

### In Regard to Constitutional Unit (A1):

The constitutional unit (a1) is a constitutional unit that contains an acid decomposable group whose polarity is increased by the action of an acid. However, those corresponding to the constitutional unit (a0) described are excluded.

The term “acid decomposable group” indicates a group having acid decomposability in which at least a part of a bond in the structure of the acid decomposable group can be cleaved by the action of an acid.

Examples of the acid decomposable group whose polarity is increased by the action of an acid include groups which are decomposed by the action of an acid to generate a polar group. Examples of the polar group include a carboxy group, a hydroxyl group, an amino group, and a sulfo group (—SO₃H).

More specific examples of the acid decomposable group include a group in which the above-described polar group has been protected with an acid dissociable group (such as a group in which a hydrogen atom of the OH-containing polar group has been protected with an acid dissociable group).

Examples of the acid dissociable group are the same as those which have been proposed as the acid dissociable groups of the base resin for a chemically amplified resist composition.

Specific examples of a group proposed as the acid dissociable group of the base resin for a chemically amplified resist composition include, among the polar groups,
an acetal type acid dissociable group which protects a carboxy group or a hydroxyl group,
a tertiary alkyl ester type acid dissociable group which protects a carboxy group,
a tertiary alkyl oxycarbonyl acid dissociable group which protects a hydroxyl group,
a secondary alkyl ester type acid dissociable group which protects a carboxy group.

Examples of the constitutional unit (a1) include a constitutional unit derived from acrylic acid ester in which the hydrogen atom bonded to the carbon atom at the α-position may be substituted with a substituent; a constitutional unit derived from acrylamide; a constitutional unit in which at least some hydrogen atoms in a hydroxyl group of a constitutional unit derived from hydroxystyrene or a hydroxystyrene derivative are protected by a substituent containing the acid decomposable group; and a constitutional unit in which at least some hydrogen atoms in —C(═O)—OH of a constitutional unit derived from vinylbenzoic acid or a vinylbenzoic acid derivative are protected by a substituent containing the acid decomposable group.

Specific examples of the constitutional unit (a1) are shown below.

In the formulae shown below, R″ represents a hydrogen atom, a methyl group, or a trifluoromethyl group.

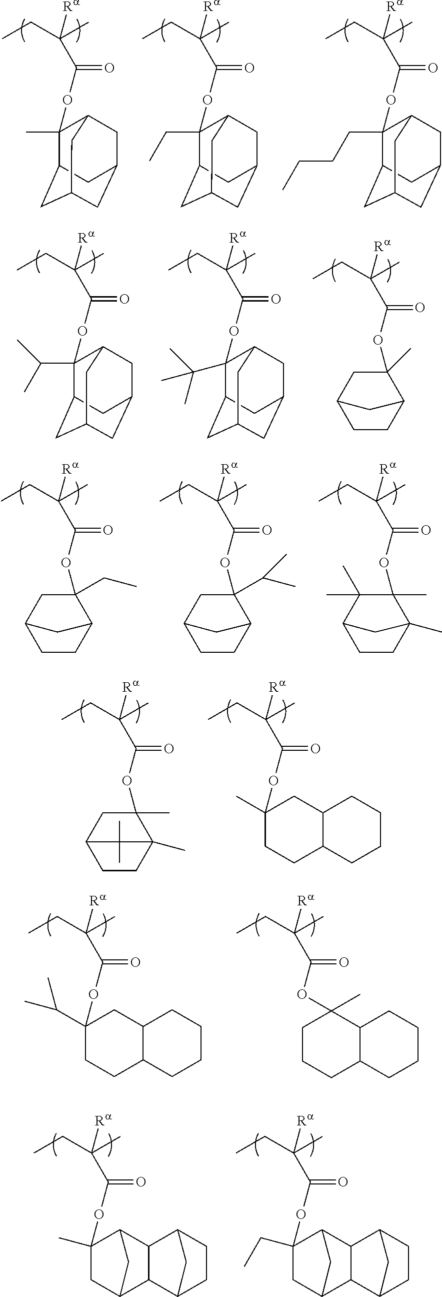

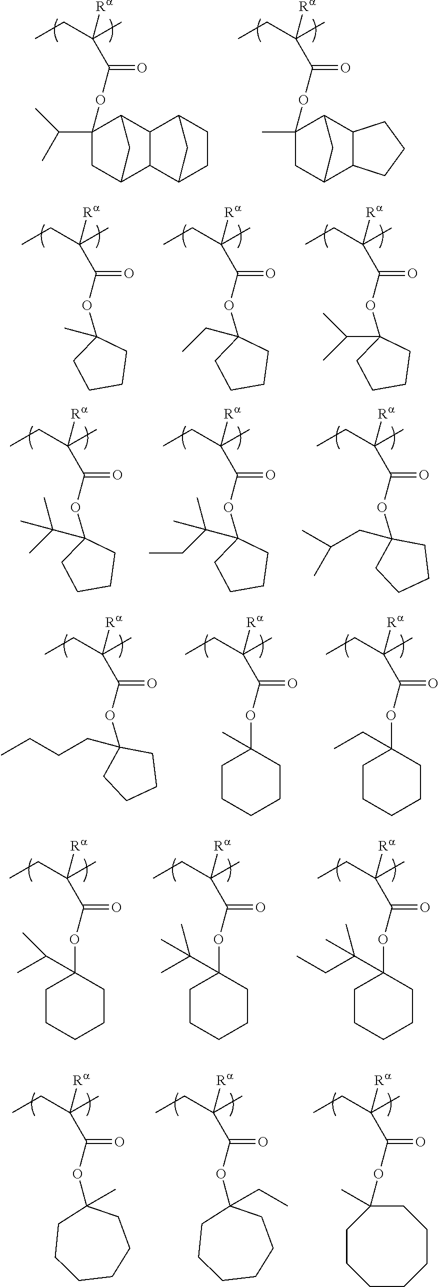

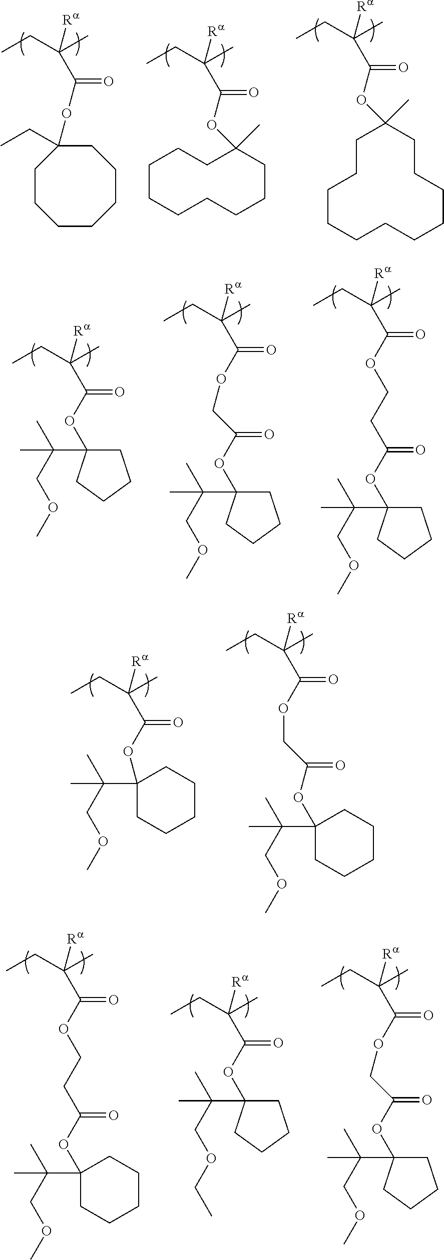

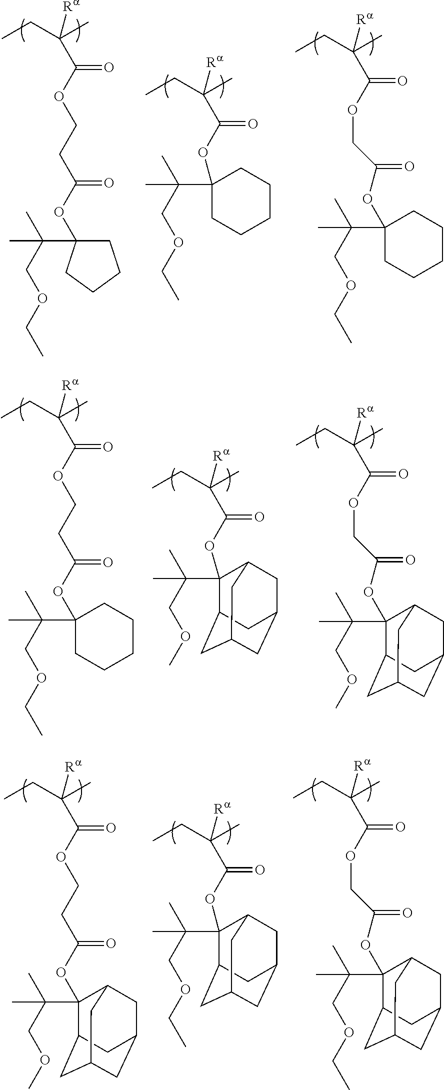

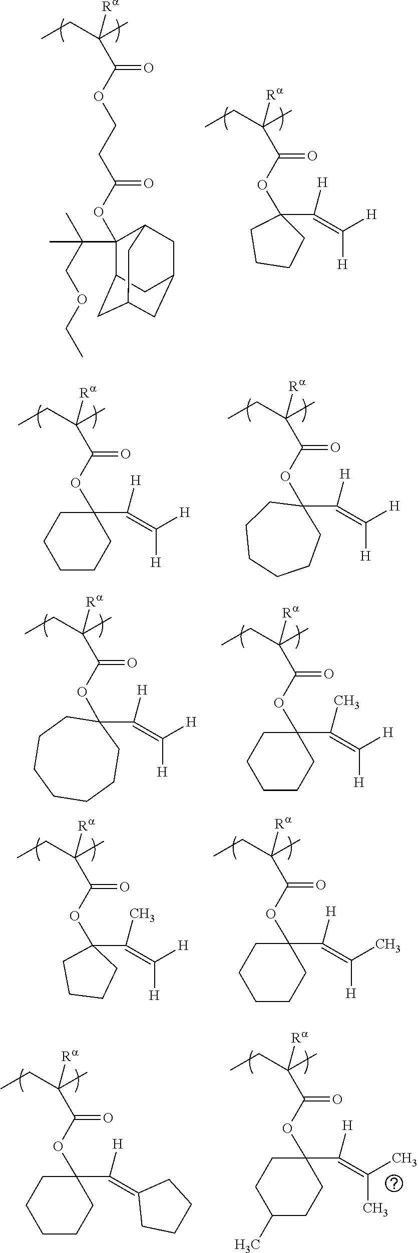

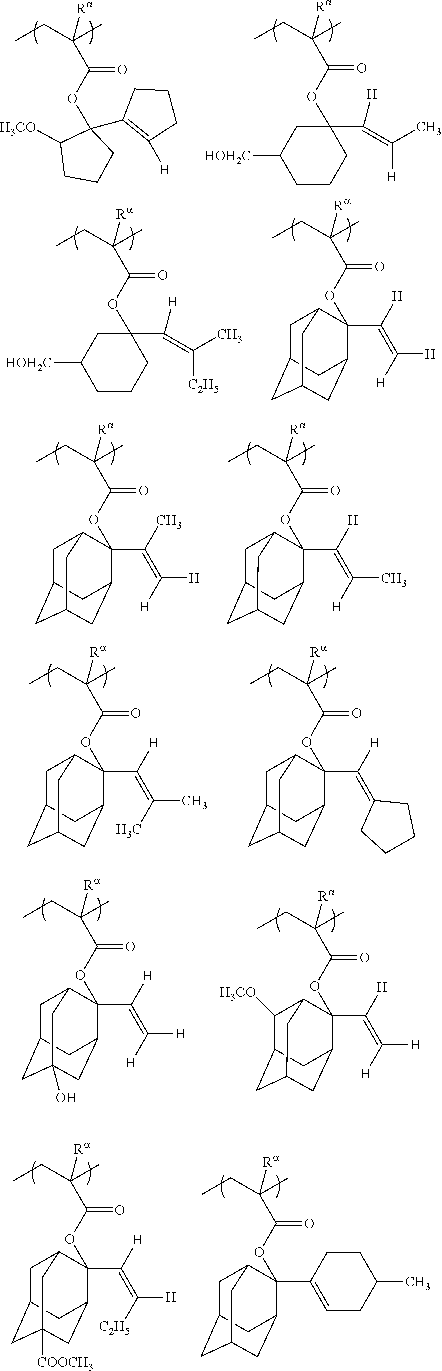

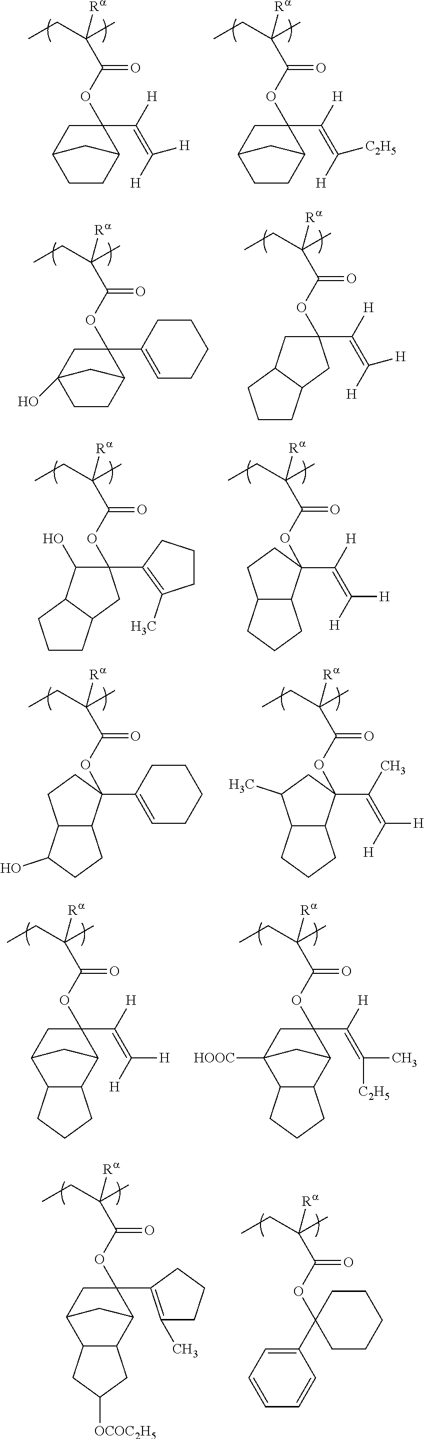

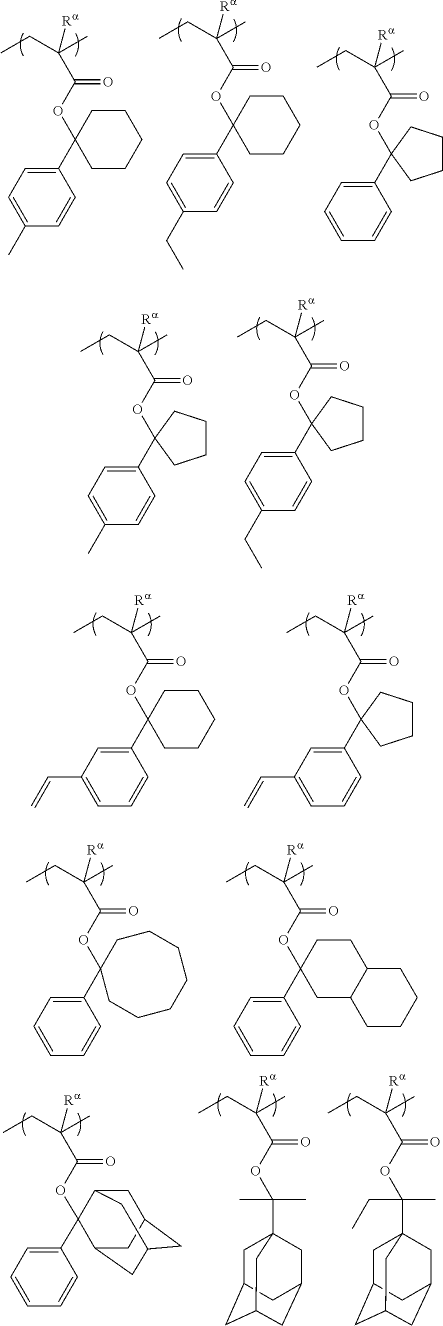

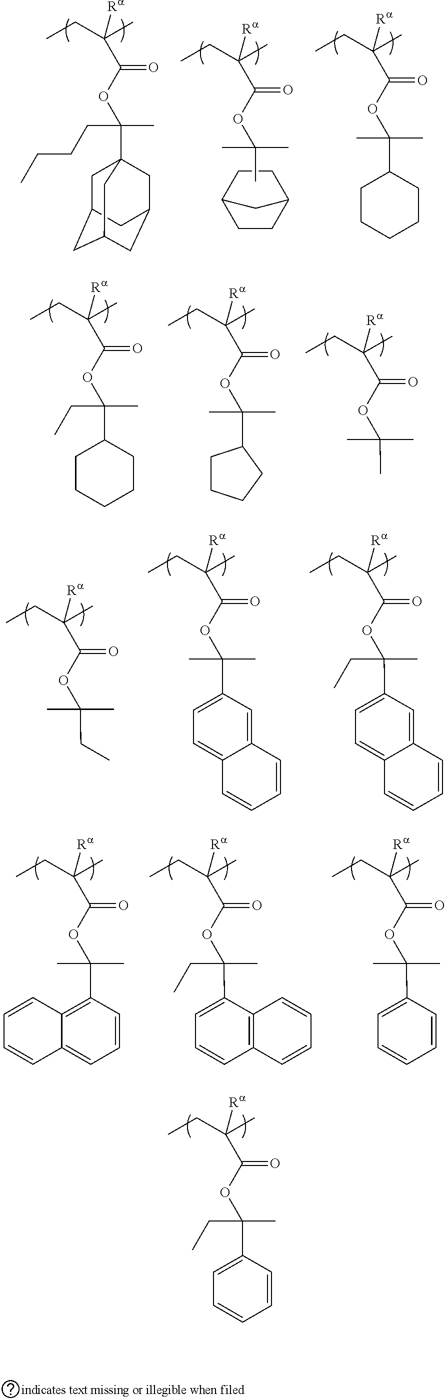

The constitutional unit (a1) included in the component (A1) may be used alone or two or more kinds thereof.

In a case where the component (A1) has the constitutional unit (a1), the proportion of the constitutional unit (a1) in the component (A1) is preferably 20% by mole or less and more preferably greater than 0% by mole and 20% by mole or less with respect to the total amount (100% by mole) of all constitutional units constituting the component (A1).

In regard to constitutional unit (a10):

The constitutional unit (a10) is represented by General Formula (a10-1). However, those corresponding to the constitutional unit (a0) described are excluded.

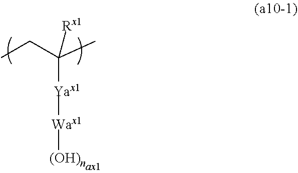

[In the formulae, R represents a hydrogen atom, an alkyl group having 1 to 5 carbon atoms, or a halogenated alkyl group having 1 to 5 carbon atoms. Ya^{x1} represents a single bond or a divalent linking group. Wa^{x1} represents an aromatic hydrocarbon group which may have a substituent, nₐₓ₁ represents an integer of 1 or greater.]

In Formula (a10-1), R represents preferably a hydrogen atom, an alkyl group having 1 to 5 carbon atoms, or a fluorinated alkyl group having 1 to 5 carbon atoms, and from the viewpoint of industrial availability, more preferably a hydrogen atom, a methyl group, or trifluoromethyl group, still more preferably a hydrogen atom or a methyl group, and particularly preferably a hydrogen atom.

In Formula (a10-1), Ya^{x1} represents a single bond or a divalent linking group.

In the chemical formula, the divalent linking group as Ya^{x1} is not particularly limited, and suitable examples thereof include a divalent hydrocarbon group which may have a substituent and a divalent linking group having a heteroatom, each of which has the same definition as that for the divalent hydrocarbon group which may have a substituent and the divalent linking group having a heteroatom, described as groups other than the polymerizable group represented by W⁰¹.

Ya^{x1} represents preferably a single bond, an ester bond [—C(═O)—O— or —O—C(=O)—], an ether bond (—O—), a linear or branched alkylene group, or a combination thereof and more preferably a single bond or an ester bond [—C(═O)—O— or —O—C(═O)—].

In Formula (a10-1), Wa^{x1} represents an aromatic hydrocarbon group which may have a substituent.

Examples of the aromatic hydrocarbon group as Wa^{x1} include a group in which (nₐₓ₁+1) hydrogen atoms have been removed from an aromatic ring which may have a substituent.

The aromatic ring here is not particularly limited as long as the aromatic ring is a cyclic conjugated system having (4n+2) n electrons. The aromatic ring has preferably 5 to 30 carbon atoms, more preferably 5 to 20 carbon atoms, still more preferably 6 to 15 carbon atoms, and particularly preferably 6 to 12 carbon atoms.

Specific examples of the aromatic ring include aromatic hydrocarbon rings such as benzene, naphthalene, anthracene, and phenanthrene; and an aromatic heterocyclic ring obtained by substituting some carbon atoms constituting the above-described aromatic hydrocarbon ring with a heteroatom. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Further, examples of the aromatic hydrocarbon group as Wa^{x1} also include a group in which (nₐₓ₁+1) hydrogen atoms have been removed from an aromatic compound having an aromatic ring (for example, biphenyl or fluorene) which may have two or more substituents.

Among the examples, Wa^{x1} represents preferably a group in which (nₐₓ₁+1) hydrogen atoms have been removed from benzene, naphthalene, anthracene, or biphenyl, more preferably a group in which (nₐₓ₁+1) hydrogen atoms have been removed from benzene or naphthalene, and still more preferably a group in which (nₐₓ₁+1) hydrogen atoms have been removed from benzene.

The aromatic hydrocarbon group as Wa^{x1} may or may not have a substituent. Examples of substituents include an alkyl group, an alkoxy group, a halogen atom, and a halogenated alkyl group.

Examples of the alkyl group, the alkoxy group, the halogen atom, and the halogenated alkyl group as the substituent include the same groups described as the substituent of the cyclic aliphatic hydrocarbon group as W⁰¹. The substituent is preferably a linear or branched alkyl group having 1 to 5 carbon atoms, more preferably a linear or branched alkyl group having 1 to 3 carbon atoms, still more preferably an ethyl group or a methyl group, and particularly preferably a methyl group.

It is preferable that the aromatic hydrocarbon group as Wa^{x1} has no substituent.

In Formula (a10-1), nₐₓ₁ represents an integer of 1 or greater, preferably an integer of 1 to 10, more preferably an integer of 1 to 5, still more preferably 1, 2, or 3, and particularly preferably 1 or 2.

Specific examples of the constitutional unit (a10) represented by Formula (a10-1) are described below.

In the formulae shown below, R^{α} represents a hydrogen atom, a methyl group, or a trifluoromethyl group.

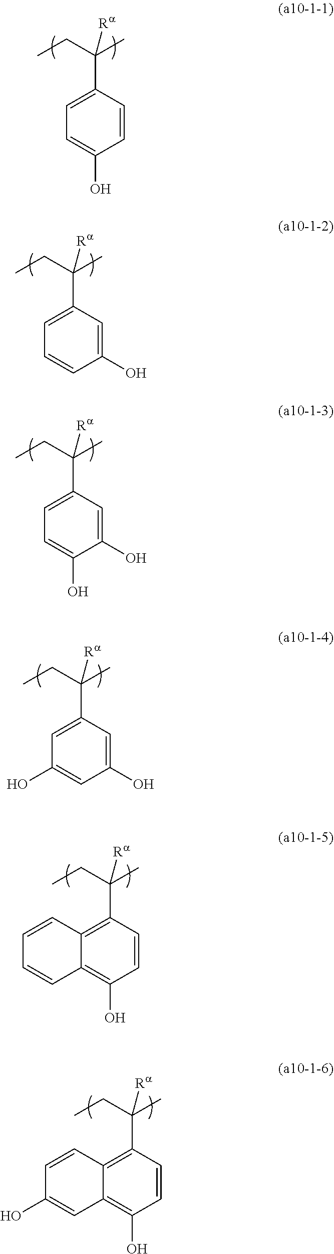

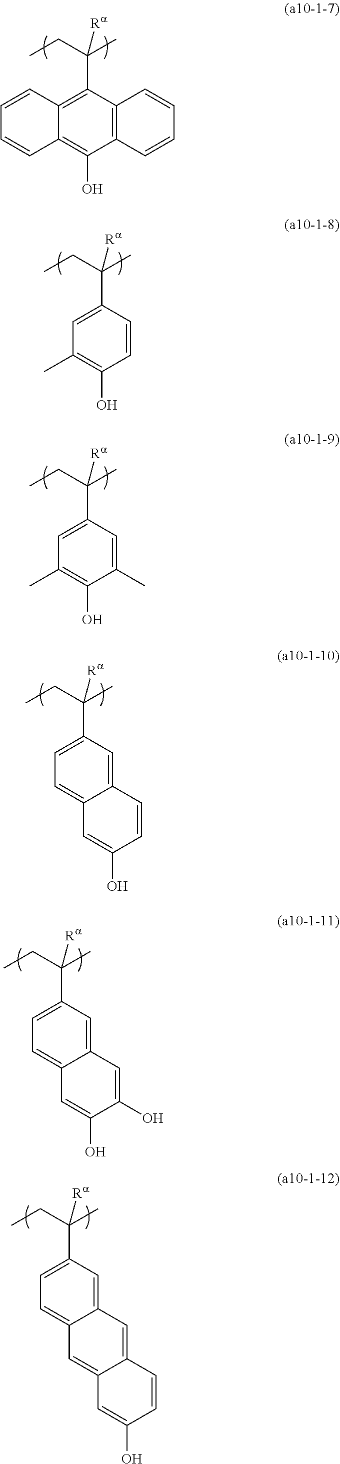

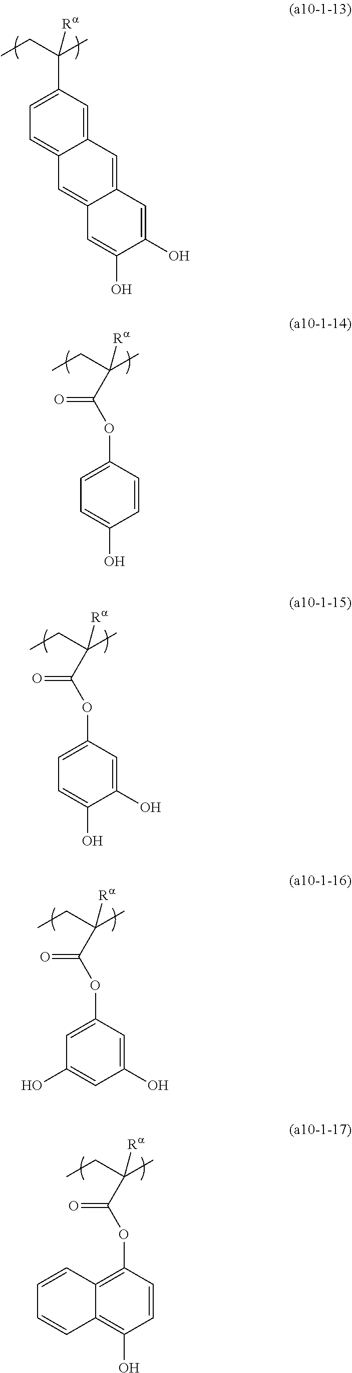

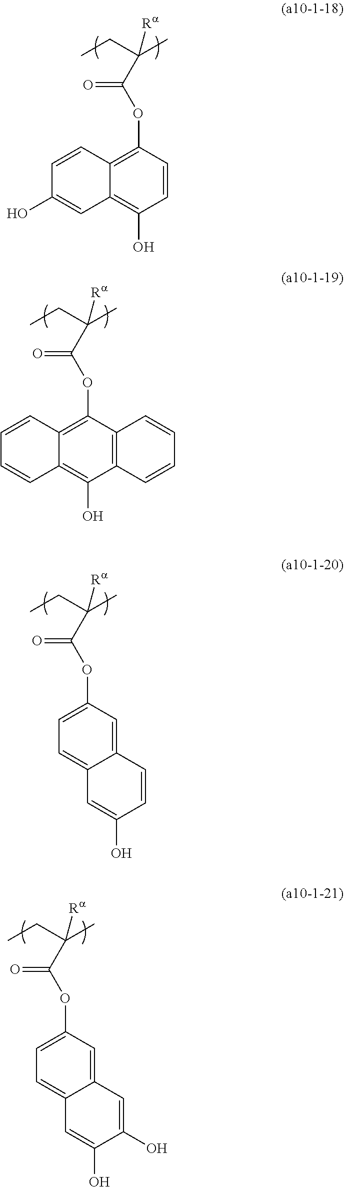

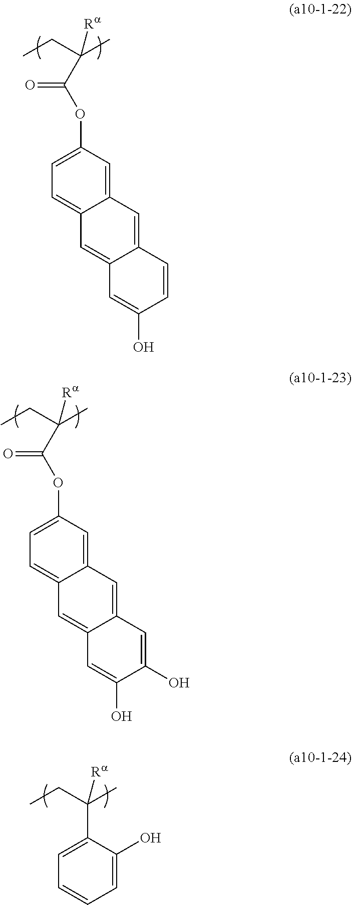

The constitutional unit (a10) included in the component (A1) may be used alone or two or more kinds thereof.

In a case where the component (A1) has the constitutional unit (a10), the proportion of the constitutional unit (a10) in the component (A1) is preferably in a range of 20% to 80% by mole, more preferably in a range of 30% to 70% by mole, and still more preferably in a range of 30% to 60% by mole with respect to the total amount (100% by mole) of all constitutional units constituting the component (A1).

In a case where the proportion of the constitutional unit (a10) is greater than or equal to the lower limits of the above-described preferable ranges, the sensitivity is likely to be further increased. Meanwhile, in a case where the proportion thereof is less than or equal to the upper limits of the above-described preferable ranges, the constitutional unit (a10) and other constitutional units are likely to be balanced.

### In Regard to Constitutional Unit (a2):

The component (A1) may further have a constitutional unit (a2) (provided that, constitutional units corresponding to the constitutional unit (a0) and the constitutional unit (a1) are excluded) containing a lactone-containing cyclic group, in addition to the constitutional unit (a0).

In a case where the component (A1) is used to form a resist film, the lactone-containing cyclic group of the constitutional unit (a2) is effective in increasing the adhesiveness of the resist film to the substrate. Further, in a case where the component (A1) contains the constitutional unit (a2), the lithography characteristics and the like are improved due to the effects of appropriately adjusting the acid diffusion length, increasing the adhesiveness of the resist film to the substrate, and appropriately adjusting the solubility during the development.

The term “lactone-containing cyclic group” indicates a cyclic group that has a ring (lactone ring) containing —O—C(═O)— in the ring skeleton. In a case where the lactone ring is counted as the first ring and the group contains only the lactone ring, the group is referred to as a monocyclic group. Further, in a case where the group has other ring structures, the group is referred to as a polycyclic group regardless of the structures. The lactone-containing cyclic group may be a monocyclic group or a polycyclic group.

The lactone-containing cyclic group in the constitutional unit (a2) is not particularly limited, and an optional constitutional unit can be used. Specific examples thereof include groups each represented by General Formulae (a2-r-1) to (a2-r-7).

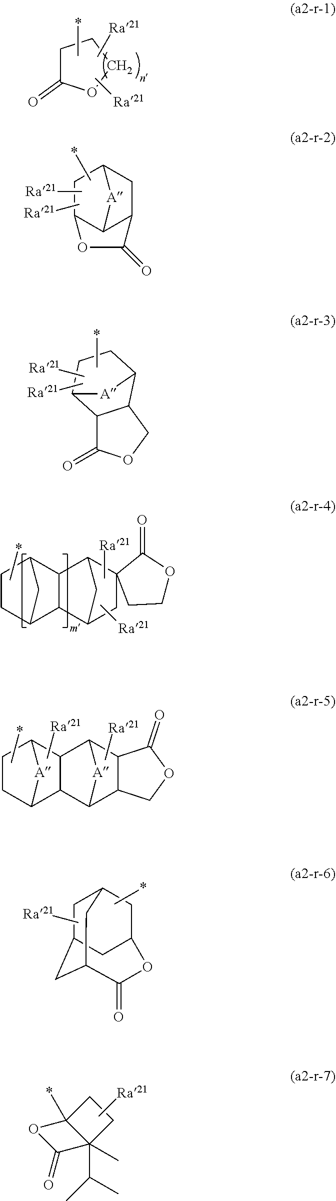

[In the formulae, a plurality of Ra′²¹'s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group, R″ represents a hydrogen atom, an alkyl group, or a lactone-containing cyclic group, A″ represents an alkylene group having 1 to 5 carbon atoms which may have an oxygen atom (—O—) or a sulfur atom (—S—), an oxygen atom, or a sulfur atom, n′ represents an integer of 0 to 2, and m′ represents 0 or 1. * represents a bonding site.]

In General Formulae (a2-r-1) to (a2-r-7), it is preferable that the alkyl group as Ra′²¹ is an alkyl group having 1 to 6 carbon atoms. It is preferable that the alkyl group is linear or branched. Specific examples thereof include a methyl group, an ethyl group, a propyl group, an isopropyl group, an n-butyl group, an isobutyl group, a tert-butyl group, a pentyl group, an isopentyl group, a neopentyl group, and a hexyl group. Among these, a methyl group or ethyl group is preferable, and a methyl group is particularly preferable.

It is preferable that the alkoxy group as Ra′²¹ is an alkoxy group having 1 to 6 carbon atoms. Further, it is preferable that the alkoxy group is linear or branched. Specific examples of the alkoxy groups include a group formed by linking the above-described alkyl group, which is an exemplary example of the alkyl group represented by Ra′²¹ to an oxygen atom (—O—).

As the halogen atom as Ra′²¹, a fluorine atom is preferable.

Examples of the halogenated alkyl group as Ra′²¹ include groups in which some or all hydrogen atoms in the alkyl group as Ra′²¹ are substituted with the halogen atoms. As the halogenated alkyl group, a fluorinated alkyl group is preferable, and a perfluoroalkyl group is particularly preferable.

In —COOR″ and —OC(═O)R″ as Ra″²¹, each R″ represents a hydrogen atom, an alkyl group, or a lactone-containing cyclic group.

The alkyl group as R″ may be linear, branched, or cyclic and has preferably 1 to carbon atoms.

In a case where R″ represents a linear or branched alkyl group, an alkyl group having 1 to 10 carbon atoms is preferable, an alkyl group having 1 to 5 carbon atoms is more preferable, and a methyl group or an ethyl group is particularly preferable.

In a case where R″ represents a cyclic alkyl group, the number of carbon atoms thereof is preferably in a range of 3 to 15, more preferably in a range of 4 to 12, and most preferably in a range of 5 to 10. Specific examples thereof can include groups in which one or more hydrogen atoms have been removed from a monocycloalkane, which may or may not be substituted with a fluorine atom or a fluorinated alkyl group; and groups in which one or more hydrogen atoms have been removed from a polycycloalkane such as bicycloalkane, tricycloalkane, or tetracycloalkane. More specific examples thereof include groups in which one or more hydrogen atoms have been removed from a monocycloalkane such as cyclopentane or cyclohexane; and groups in which one or more hydrogen atoms have been removed from a polycycloalkane such as adamantane, norbornane, isobornane, tricyclo[5.2.1.0^{2,6}]decane, or tetracyclododecane.

Examples of the lactone-containing cyclic group as R″include the same groups as those for the groups each represented by General Formulae (a2-r-1) to (a2-r-7).

As the hydroxyalkyl group as Ra′²¹, a hydroxyalkyl group having 1 to 6 carbon atoms is preferable, and specific examples thereof include a group in which at least one hydrogen atom in the alkyl group as Ra′²¹ has been substituted with a hydroxyl group.

Among the examples, it is preferable that Ra′²¹'s each independently represent a hydrogen atom or a cyano group.

In General Formulae (a2-r-2), (a2-r-3) and (a2-r-5), as the alkylene group having 1 to 5 carbon atoms as A″, a linear or branched alkylene group is preferable, and examples thereof include a methylene group, an ethylene group, an n-propylene group, and an isopropylene group. In a case where the alkylene group has an oxygen atom or a sulfur atom, specific examples thereof include groups in which —O— or —S— is interposed in the terminal of the alkylene group or between the carbon atoms of the alkylene group. Further, examples thereof include —O—CH₂—, —CH₂-0-CH₂—, —S—CH₂—, and —CH₂—S—CH₂—. A″ represents preferably an alkylene group having 1 to 5 carbon atoms or —O—, more preferably an alkylene group having 1 to 5 carbon atoms, and most preferably a methylene group.

Specific examples of the groups each represented by General Formulae (a2-r-1) to (a2-r-7) are shown below.

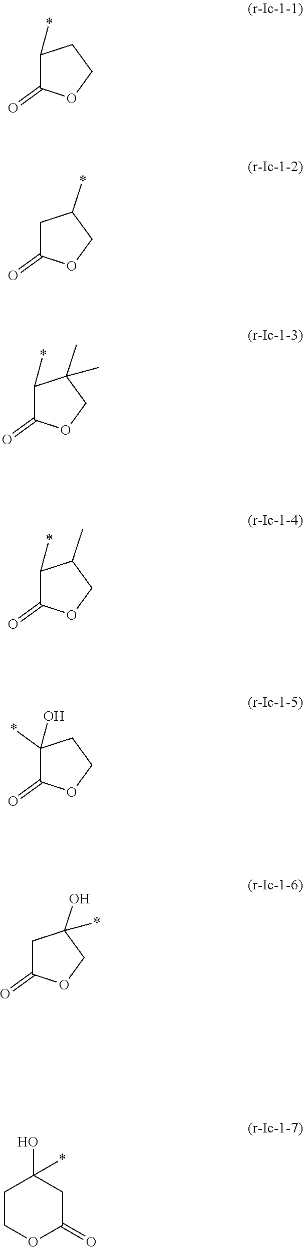

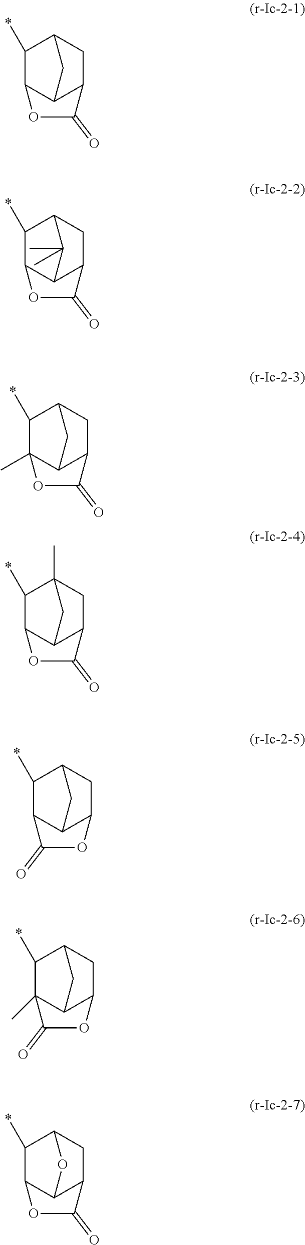

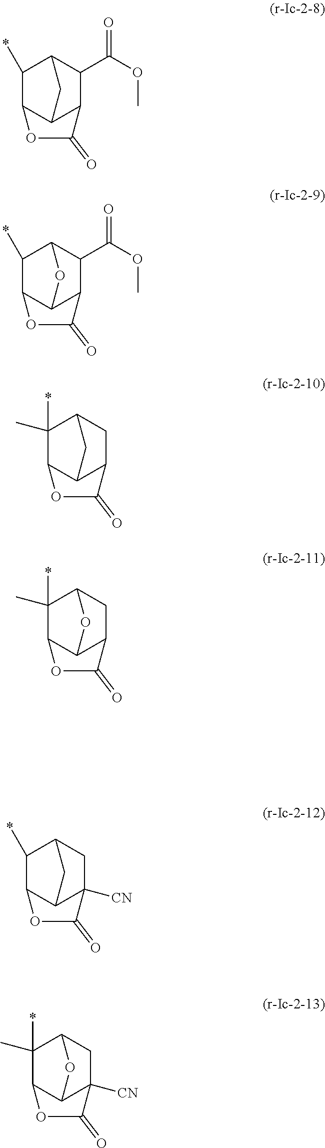

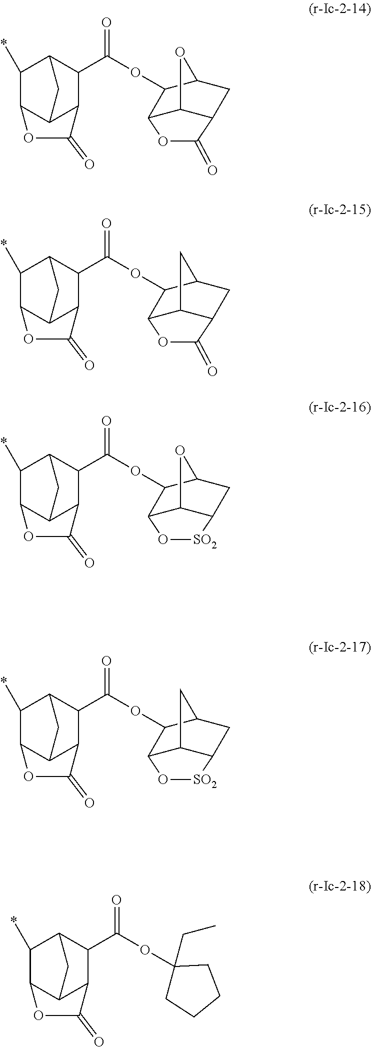

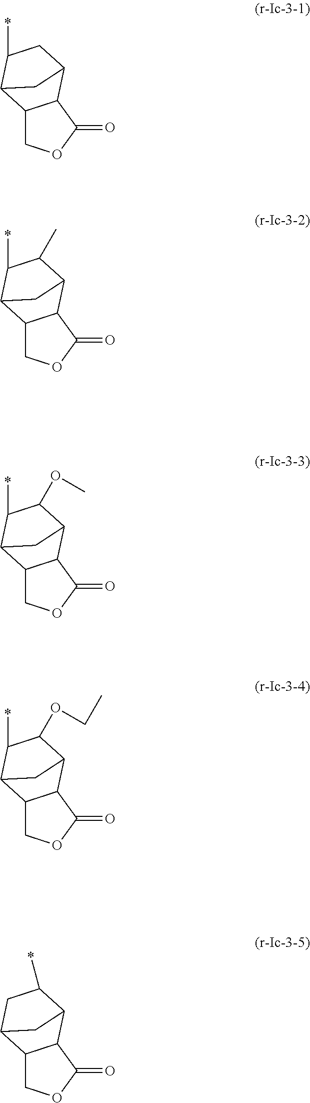

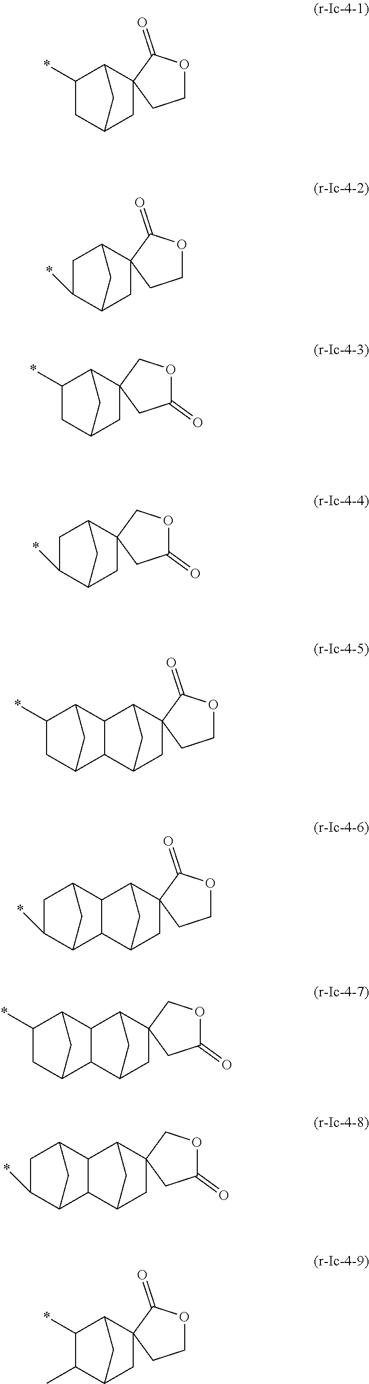

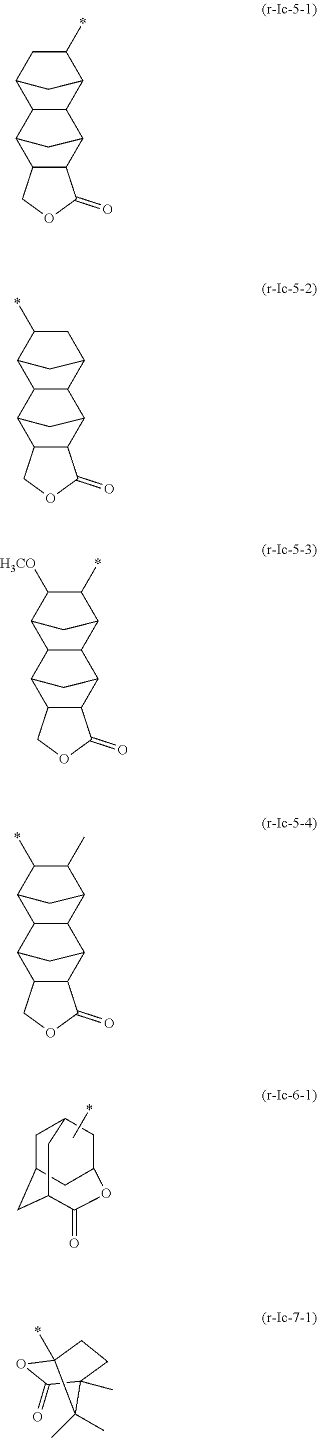

As the constitutional unit (a2), a constitutional unit derived from acrylic acid ester in which the hydrogen atom bonded to the carbon atom at the α-position may be substituted with a substituent is preferable.

It is preferable that such a constitutional unit (a2) is represented by General Formula (a2-1).

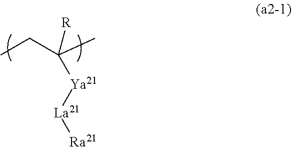

[in the formula, R represents a hydrogen atom, an alkyl group having 1 to 5 carbon atoms, or a halogenated alkyl group having 1 to 5 carbon atoms. Ya²¹ represents a single bond or a divalent linking group. La²¹ represents —O—, —COO—, —CON(R′)—, —OCO—, —CONHCO—, or —CONHCS—, and R′ represents a hydrogen atom or a methyl group. Provided that, in a case where La²¹ represents —O—, Ya²¹ does not represent —CO—. Ra²¹ represents a lactone-containing cyclic group.]

In Formula (a2-1), R has the same definition as described above. R represents preferably a hydrogen atom, an alkyl group having 1 to 5 carbon atoms, or a fluorinated alkyl group having 1 to 5 carbon atoms and particularly preferably a hydrogen atom or a methyl group from the viewpoint of the industrial availability.

In Formula (a2-1), the divalent linking group as Ya²¹ is not particularly limited, and suitable examples thereof include a divalent hydrocarbon group which may have a substituent and a divalent linking group having a heteroatom, each of which has the same definition as the divalent hydrocarbon group which may have a substituent and the divalent linking group having a heteroatom, described as groups other than the polymerizable group represented by W⁰¹.

Ya²¹ represents preferably a single bond, an ester bond [—C(═O)—O—], an ether bond (—O—), a linear or branched alkylene group, or a combination thereof and particularly preferably a single bond.

In Formula (a2-1), it is preferable that Ya²¹ represents a single bond and La²¹ represents —COO— or —OCO—.

In Formula (a2-1), Ra²¹ represents a lactone-containing cyclic group.

Suitable examples of the lactone-containing cyclic group as Ra²¹ include groups each represented by General Formulae (a2-r-1) to (a2-r-7).

Among these, groups each represented by General Formula (a2-r-1) or (a2-r-2) are preferable. Specifically, groups each represented by Chemical Formulae (r-1c-1-1) to (r-1c-1-7) and (r-1c-2-1) to (r-1c-2-18) are preferable, and groups each represented by Chemical Formulae (r-1c-1-1), (r-1c-2-1), and (r-1c-2-7) are more preferable.

The constitutional unit (a2) included in the component (A1) may be used alone or two or more kinds thereof.

In a case where the component (A1) has the constitutional unit (a2), the proportion of the constitutional unit (a2) is preferably greater than 0% by mole and 20% by mole or less with respect to the total amount (100% by mole) of all constitutional units constituting the component (A1).

In a case where the proportion of the constitutional unit (a2) is greater than or equal to the lower limits of the above-described preferable ranges, the effect obtained by allowing the component (A1) to have the constitutional unit (a2) can be sufficiently achieved. Meanwhile, in a case where the proportion thereof is less than or equal to the above-described upper limits of the above-described preferable ranges, the constitutional unit (a2) and other constitutional units can be balanced, and various lithography characteristics are enhanced.

### In Regard to Constitutional Unit (a8):

The constitutional unit (a8) is a constitutional unit derived from a compound represented by General Formula (a8-1). Provided that, constitutional units corresponding to the constitutional unit (a0) are excluded.

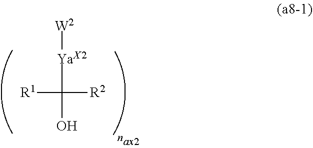

[In the formula. W² represents a polymerizable group-containing group. Ya^{x2} represents a single bond or an (nₓ₂+1)-valent linking group. Ya^{x2} and W² may form a condensed ring. R¹ represents a fluorinated alkyl group having 1 to 12 carbon atoms. R² represents an organic group having 1 to 12 carbon atoms which may have a fluorine atom or a hydrogen atom. R² and Ya^{x2} may be bonded to each other to form a ring structure, nₐₓ₂ represents an integer of 1 to 3.1

The description of the polymerizable group-containing group as W² in Formula (a8-1) is the same as the description of the polymerizable group-containing group as W⁰¹ in General Formula (a0-1).

Suitable examples of the polymerizable group-containing group as W² include a group represented by Chemical Formula: C(R^{X11})(R^{X12})═C(R^{Xl};)—Ya^{x0}—.

In the chemical formula, R^{X11}, R^{X12}, and R^{X13} each represent a hydrogen atom, an alkyl group having 1 to 5 carbon atoms, or a halogenated alkyl group having 1 to 5 carbon atoms, and Ya^{x0} represents a single bond or a divalent linking group and has the same definition as that for the group represented by Chemical Formula: C(R^{X11})(R^{X12})═C(R^{X13})—Ya^{x0}—described in the section of W⁰¹ in General Formula (a0-1).

Specific examples of the constitutional unit (a8) are shown below.

In the following formulae, R′ represents a hydrogen atom, a methyl group, or a trifluoromethyl group.

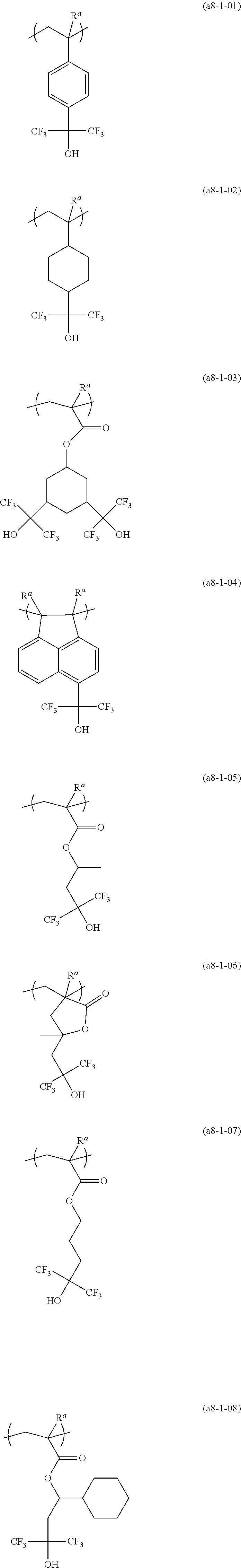

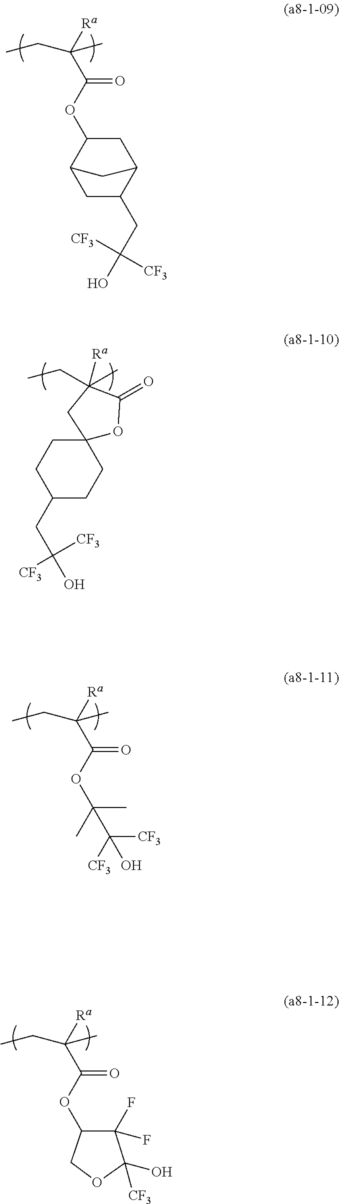

The constitutional unit (a8) contained in the component (A1) may be used alone or two or more kinds thereof.

In a case where the component (A1) contains the constitutional unit (a8), the proportion of the constitutional unit (a8) is preferably greater than 0% by mole and 20% by mole or less with respect to the total amount (100% by mole) of all constitutional units constituting the component (A1).

The component (A1) contained in the resist composition may be used alone or in combination of two or more kinds thereof.

In the resist composition according to the present embodiment, the component (A1) includes a polymer compound having a repeating structure of the constitutional unit (a0).

Among the examples, suitable examples of the component (A1) include a polymer compound having a repeating structure of the constitutional unit (a0) and the constitutional unit (a10).

More specific suitable examples of the polymer compound contained in the component (A1) include a polymer compound having a repeating structure of the constitutional unit (a0) and the constitutional unit (a10); a polymer compound having a repeating structure of the constitutional unit (a), the constitutional unit (a10), and the constitutional unit (a1); and a polymer compound having a repeating structure of the constitutional unit (a0), the constitutional unit (a10), and the constitutional unit (a2).

Such a component (A1) can be produced by dissolving a monomer, from which each constitutional unit is derived, in a polymerization solvent and adding a radical polymerization initiator such as azobisisobutyronitrile (AIBN) or dimethyl azobisisobutyrate (for example, V-601) to the solution so that the polymerization is carried out.

Alternatively, the component (A1) can be produced by dissolving, in a polymerization solvent, a monomer from which the constitutional unit (a0) is derived and, as necessary, a monomer from which a constitutional unit (for example, the constitutional unit (a10)) other than the constitutional unit (a0) is derived, adding thereto a radical polymerization initiator as described above to carry out polymerization, and then carrying out a deprotection reaction.

Further, a —C(CF₃)₂—OH group may be introduced to the terminal during the polymerization using a combination of chain transfer agents such as HS—CH₂—CH₂—CH₂—C(CF₃)₂—OH. As described above, a copolymer into which a hydroxyalkyl group, formed by substitution of some hydrogen atoms in the alkyl group with fluorine atoms, has been introduced is effective for reducing development defects and reducing line edge roughness (LER: uneven irregularities of a line side wall).

The weight-average molecular weight (Mw) (in terms of polystyrene according to gel permeation chromatography (GPC)) of the component (A1) is not particularly limited, but is preferably in a range of 1000 to 50000, more preferably in a range of 2000 to 30000, and still more preferably in a range of 3000 to 20000.

In a case where the Mw of the component (A1) is less than or equal to the upper limits of the above-described preferable ranges, a satisfactory solubility in a resist solvent for a resist enough to be used as a resist is exhibited. On the contrary, in a case where the Mw of the component (A1) is greater than or equal to the lower limits of the above-described preferable ranges, the dry etching resistance and the cross-sectional shape of the resist pattern are excellent.

The dispersity (Mw/Mn) of the component (A1) is not particularly limited; however, it is preferably in a range of 1.0 to 3.0, more preferably in a range of 1.0 to 2.5, and particularly preferably in a range of 1.0 to 2.0. Further, Mn represents the number average molecular weight.

### In Regard to Component (A2)

In the resist composition of the present embodiment, a base material component (hereinafter, also referred to as “component (A2)”) which does not correspond to the component (A1) and whose solubility in a developing solution is changed by the action of an acid may be used in combination as the component (A).

The component (A2) is not particularly limited and may be optionally selected from a plurality of components of the related art which have been known as base material components for a chemically amplified resist composition and used.

As the component (A2), a polymer compound or a low-molecular-weight compound may be used alone or in combination of two or more kinds thereof.

The proportion of the component (A1) in the component (A) is preferably 25% by mass or greater, more preferably 50% by mass or greater, and still more preferably 75% by mass or greater, and may be 100% by mass with respect to the total mass of the component (A). In a case where the proportion thereof is 25% by mass or greater, a resist pattern having excellent various lithography characteristics such as high sensitivity, high resolution, and the property of suppressing film reduction, and improved roughness is likely to be formed.

In the resist composition of the present embodiment, the content of the component (A) may be adjusted according to the thickness of the resist film intended to be formed.

### <Other Components>

The resist composition according to the present embodiment may further contain other components in addition to the component (A) described above. Examples of the other components include a component (B), a component (D), a component (E), a component (F), and a component (S), which are described below.

### <<Acid Generator Component (B)>>

It is preferable that the resist composition according to the present embodiment further contains an acid generator component (B) that generates an acid upon light exposure.

The component (B) is not particularly limited, and those which have been proposed so far as an acid generator for a chemically amplified resist composition in the related art can be used.

Examples of the acid generator include various acid generators, for example, onium salt-based acid generators such as iodonium salts and sulfonium salts; oxime sulfonate-based acid generators; diazomethane-based acid generators such as bisalkyl or bisaryl sulfonyl diazomethanes and poly(bis-sulfonyl)diazomethanes; nitrobenzyl sulfonate-based acid generators, iminosulfonate-based acid generators, and disulfone-based acid generators.

Examples of the onium salt-based acid generators include a compound represented by General Formula (b-1) (hereinafter, also referred to as “component (b-1)”), a compound represented by General Formula (b-2) (hereinafter, also referred to as “component (b-2)”), and a compound represented by General Formula (b-3) (hereinafter, also referred to as “component (b-3)”).

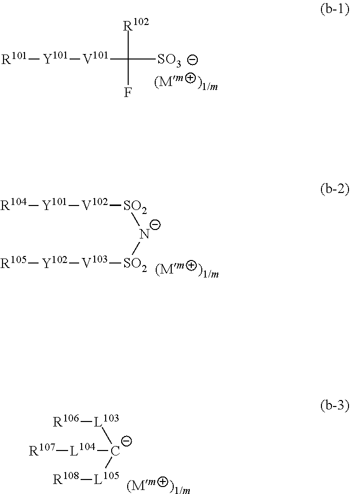

[In the formulae, R¹⁰¹ and R¹⁰⁴ to R¹⁰⁸ each independently represent a cyclic group which may have a substituent, a chain-like alkyl group which may have a substituent, or a chain-like alkenyl group which may have a substituent. R¹⁰⁴ and R¹⁰⁵ may be bonded to each other to form a ring structure. R¹⁰² represents a fluorinated alkyl group having 1 to 5 carbon atoms or a fluorine atom. Y¹⁰¹ represents a divalent linking group having an oxygen atom or a single bond. V¹⁰¹ to V¹⁰³ each independently represent a single bond, an alkylene group, or a fluorinated alkylene group. L¹⁰¹ and L¹⁰² each independently represent a single bond or an oxygen atom. L¹⁰³ to L¹⁰⁵ each independently represent a single bond, —CO—, or —SO₂—, in represents an integer of 1 or greater, and M′^{m′} represents an m-valent onium cation.]

### {Anion Moiety}

Anions in Component (b-1)

In Formula (b-1), R¹⁰¹ represents a cyclic group which may have a substituent, a chain-like alkyl group which may have a substituent, or a chain-like alkenyl group which may have a substituent.

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group with no aromaticity. Further, the aliphatic hydrocarbon group may be saturated or unsaturated. In general, it is preferable that the aliphatic hydrocarbon group is saturated.

The aromatic hydrocarbon group as R¹⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Provided that, the number of carbon atoms in a substituent is not included in the number of carbon atoms.

Specific examples of the aromatic ring of the aromatic hydrocarbon group as R¹⁰¹ include benzene, fluorene, naphthalene, anthracene, phenanthrene, biphenyl, and an aromatic heterocyclic ring in which some carbon atoms constituting any of these aromatic rings are substituted with heteroatoms. Examples of the heteroatom in the aromatic heterocyclic rings include an oxygen atom, a sulfur atom, and a nitrogen atom.

Specific examples of the aromatic hydrocarbon group as R¹⁰¹ include a group in which one hydrogen atom has been removed from the aromatic ring (an aryl group such as a phenyl group or a naphthyl group) and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R¹⁰¹ include an aliphatic hydrocarbon group having a ring in the structure thereof.

Examples of the aliphatic hydrocarbon group having a ring in the structure thereof include an alicyclic hydrocarbon group (a group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring polycyclic skeleton such as adamantane, norbornane, isobornane, tricyclo[5.2.1.0^{2,6}]decane, or tetracyclododecane; and a polycycloalkane having a condensed ring polycyclic skeleton such as a cyclic group having a steroid skeleton are preferable as the polycycloalkane.

Among these examples, as the cyclic aliphatic hydrocarbon group as R¹⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is still more preferable, and an adamantyl group is particularly preferable.

The linear aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and most preferably 1 to 3 carbon atoms. As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅-].

The branched aliphatic hydrocarbon group which may be bonded to the alicyclic hydrocarbon group has preferably 2 to 10 carbon atoms, more preferably 3 to 6 carbon atoms, still more preferably 3 or 4 carbon atoms, and most preferably 3 carbon atoms. As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂H₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

Further, the cyclic hydrocarbon group as R¹⁰¹ may have a heteroatom such as a heterocyclic ring. Specific examples thereof include lactone-containing cyclic groups each represented by General Formulae (a2-r-1) to (a2-r-7), heterocyclic groups each represented by Chemical Formulae (r-hr-1) to (r-hr-16), and —SO₂-containing cyclic groups each represented by General Formulae (b5-r-1) to (b5-r-4).

In the formulae, * represents a bonding site with respect to Y¹⁰¹ in Formula (b-1).

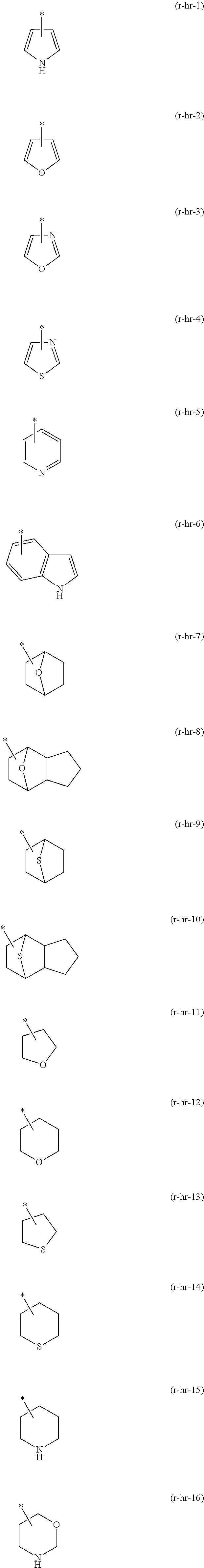

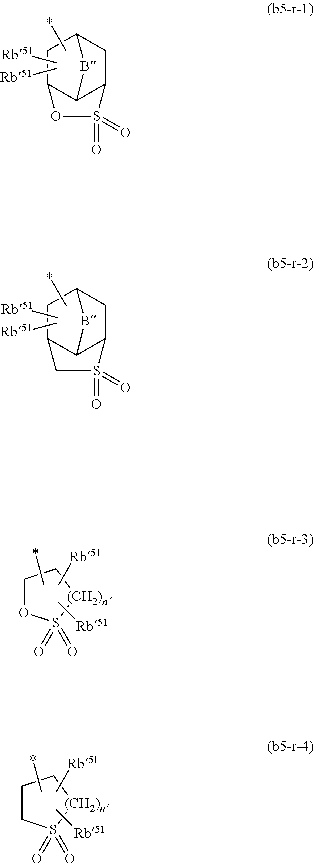

[In the formulae, Rb′⁵¹'s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group, R″ represents a hydrogen atom, an alkyl group, a lactone-containing cyclic group, or a —SO₂-containing cyclic group, B″ represents an alkylene group having 1 to 5 carbon atoms which may have an oxygen atom or a sulfur atom, an oxygen atom, or a sulfur atom, and n′ represents an integer of 0 to 2. * represents a bonding site.]

In General Formulae (b5-r-1) and (b5-r-2), B″ represents an alkylene group having 1 to 5 carbon atoms which may have an oxygen atom or a sulfur atom, an oxygen atom, or a sulfur atom.

B″ represents preferably an alkylene group having 1 to 5 carbon atoms or —O—, more preferably an alkylene group having 1 to 5 carbon atoms, and still more preferably a methylene group.

In General Formulae (b5-r-1) to (b5-r-4), Rb's” 's each independently represent a hydrogen atom, an alkyl group, an alkoxy group, a halogen atom, a halogenated alkyl group, a hydroxyl group, —COOR″, —OC(═O)R^{αx}, a hydroxyalkyl group, or a cyano group. Among these, it is preferable that Rb′⁵¹'s each independently represent a hydrogen atom or a cyano group.

Specific examples of the groups each represented by General Formulae (b5-r-1) to (b5-r-4) are shown below. In the formulae shown below, “Ac” represents an acetyl group.

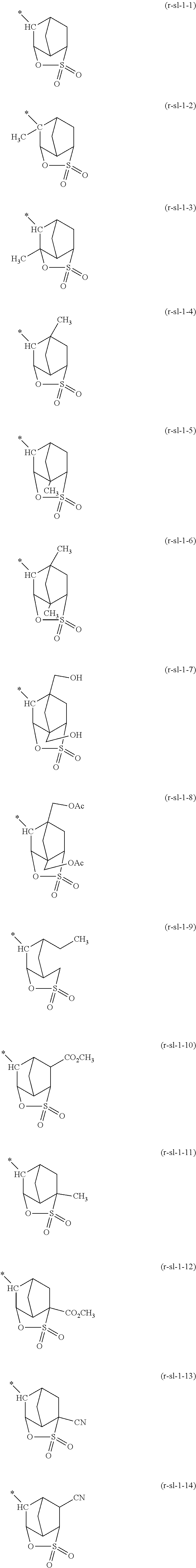

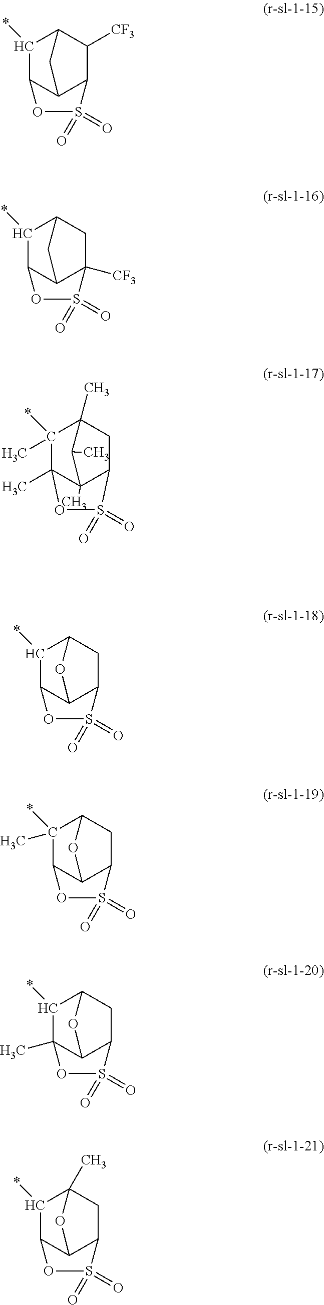

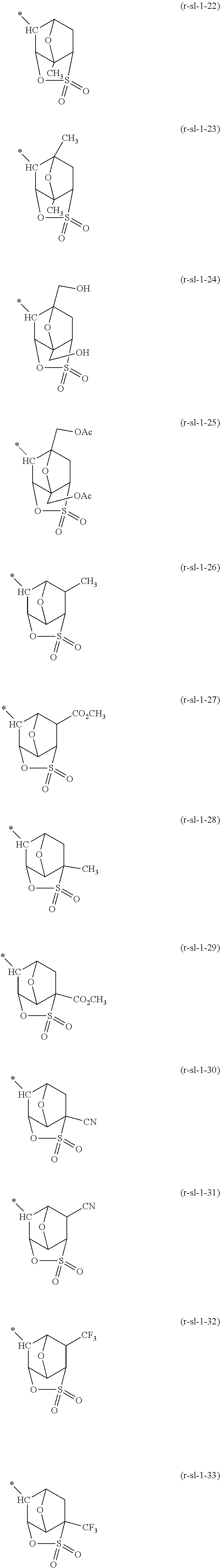

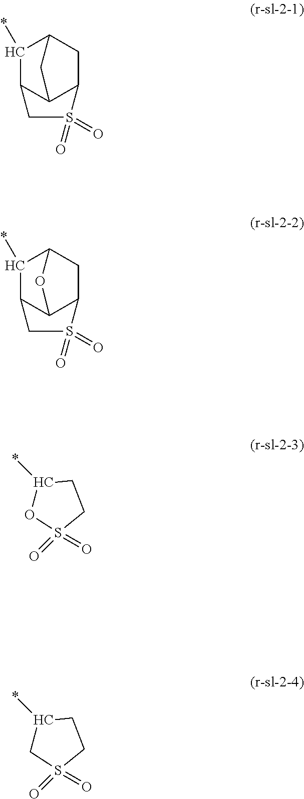

Examples of the substituent for the cyclic group as R¹⁰¹ include an alkyl group, an alkoxy group, a halogen atom, a halogenated alkyl group, a hydroxyl group, a carbonyl group, and a nitro group.

As the alkyl group as the substituent, an alkyl group having 1 to 5 carbon atoms is preferable, and a methyl group, an ethyl group, a propyl group, an n-butyl group, or a tert-butyl group is most preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

Examples of the halogen atom as the substituent include a fluorine atom, a chlorine atom, a bromine atom, and an iodine atom. Among these, a bromine atom and an iodine atom are preferable, and an iodine atom is more preferable. Example of the above-described halogenated alkyl group as the substituent includes a group in which some or all hydrogen atoms in an alkyl group having 1 to 5 carbon atoms such as a methyl group, an ethyl group, a propyl group, an n-butyl group, or a tert-butyl group have been substituted with the above-described halogen atoms. The carbonyl group as the substituent is a group that substitutes a methylene group (—CH₂—) constituting the cyclic hydrocarbon group.

The cyclic hydrocarbon group as R¹⁰¹ may be a condensed cyclic group having a condensed ring in which an aliphatic hydrocarbon ring and an aromatic ring are condensed. Examples of the condensed ring include those obtained by condensing one or more aromatic rings with a polycycloalkane having a crosslinked ring polycyclic skeleton. Specific examples of the crosslinked ring polycycloalkane include a bicycloalkane such as bicyclo[2.2.1]heptane (norbornane) and bicyclo[2.2.2]octane. As the condensed cyclic group, a group having a condensed ring in which two or three aromatic rings are condensed with a bicycloalkane is preferable, and a group having a condensed ring in which two or three aromatic rings are condensed with bicyclo[2.2.2]octane is more preferable. Specific suitable examples of the condensed cyclic group as R¹⁰¹ include groups represented by Formulae (r-br-1) and (r-br-2).

In the formulae, * represents a bonding site with respect to Y¹⁰¹ in Formula (b-1).

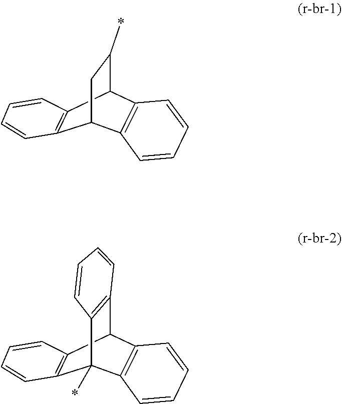 [SMILES: *C1CC2c3ccccc3C1c1ccccc12 ; *C12c3ccccc3C(c3ccccc31)c1ccccc12]

Examples of the substituent that the condensed cyclic group as R¹⁰¹ may have include an alkyl group, an alkoxy group, a halogen atom, a halogenated alkyl group, a hydroxyl group, a carbonyl group, a nitro group, an aromatic hydrocarbon group, and an alicyclic hydrocarbon group.

Examples of the alkyl group, the alkoxy group, the halogen atom, and the halogenated alkyl group as the substituent of the condensed cyclic group include those described as the substituent of the cyclic group as R¹⁰¹.

Examples of the aromatic hydrocarbon group as the substituent of the condensed cyclic group include a group in which one hydrogen atom has been removed from the aromatic ring (an aryl group such as a phenyl group or a naphthyl group), a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group), and a heterocyclic group represented by each of Chemical Formulae (r-hr-1) to (r-hr-6).

Examples of the alicyclic hydrocarbon group as the substituent of the condensed cyclic group include a group in which one hydrogen atom has been removed from a monocycloalkane such as cyclopentane or cyclohexane, a group in which one hydrogen atom has been removed from a polycycloalkane such as adamantane, norbornane, isobornane, tricyclo[5.2.1.0^{2,6}]decane, or tetracyclododecane, a lactone-containing cyclic group represented by each of General Formulae (a2-r-1) to (a2-r-7), a —SO₂-containing cyclic group represented by each of General Formulae (b5-r-1) to (b5-r-4), and a heterocyclic group represented by each of Chemical Formulae (r-hr-7) to (r-hr-16).

Chain-Like Alkyl Group which May have Substituent:

The chain-like alkyl group as R¹⁰¹ may be linear or branched.

The linear alkyl group has preferably 1 to 20 carbon atoms, more preferably 1 to 15 carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a I-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-Like Alkenyl Group which May have Substituent:

The chain-like alkenyl group as R¹⁰¹ may be linear or branched, and the number of carbon atoms thereof is preferably in a range of 2 to 10, more preferably in a range of 2 to 5, still more preferably in a range of 2 to 4, and particularly preferably 3. Examples of the linear alkenyl group include a vinyl group, a propenyl group (an allyl group), and a butynyl group. Examples of the branched alkenyl group include a 1-methylvinyl group, a 2-methylvinyl group, a 1-methylpropenyl group, and a 2-methylpropenyl group.

Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R¹⁰¹ include an alkoxy group, a halogen atom, a halogenated alkyl group, a hydroxyl group, a carbonyl group, a nitro group, an amino group, and a cyclic group as R¹⁰¹.

Among the examples, R¹⁰¹ represents preferably a cyclic group which may have a substituent and more preferably a cyclic hydrocarbon group which may have a substituent.

More specifically, as the cyclic hydrocarbon group, an aromatic hydrocarbon group, a group in which one or more hydrogen atoms have been removed from a polycycloalkane, a lactone-containing cyclic group represented by each of General Formulae (a2-r-1) to (a2-r-7), a —S02-containing cyclic group represented by each of General Formulae (b5-r-1) to (b5-r-4), and a condensed cyclic group having a condensed ring in which an aliphatic hydrocarbon ring and an aromatic ring are condensed are preferable, and an aromatic hydrocarbon group and a condensed cyclic group having a condensed ring in which an aliphatic hydrocarbon ring and an aromatic ring are condensed are more preferable.

Among these, it is still more preferable that R¹¹¹ represents an alkyl group having 1 to 5 carbon atoms, an aryl group having a substituent selected from the group consisting of a bromine atom and an iodine atom, or a group represented by any of Formulae (r-br-1) and (r-br-2).

In Formula (b-1), Y¹⁰¹ represents a divalent linking group having an oxygen atom or a single bond.

In a case where Y¹⁰¹ represents a divalent linking group containing an oxygen atom, Y¹⁰¹, may contain an atom other than the oxygen atom. Examples of atoms other than an oxygen atom include a carbon atom, a hydrogen atom, a sulfur atom, and a nitrogen atom.

Examples of the divalent linking group having an oxygen atom include a non-hydrocarbon oxygen atom-containing linking group such as an oxygen atom (an ether bond: —O—), an ester bond (—C(═O)—O—), an oxycarbonyl group (—O—C(═O)—), an amide bond (—C(═O)—NH—), a carbonyl group (—C(═O)—), or a carbonate bond (—O—C(═O)—O—); and combinations of the above-described non-hydrocarbon oxygen atom-containing linking groups with an alkylene group. Further, a sulfonyl group (—SO₂—) may be further linked to the combination. Examples of the divalent linking group having an oxygen atom include linking groups each represented by General Formulae (y-a1-1) to (y-a1-7).

Further, in General Formulae (y-a1-1) to (y-a1-7), V′¹⁰¹ in General Formulae (y-a1-1) to (y-a1-7) is bonded to R¹⁰¹ in Formula (b-1).

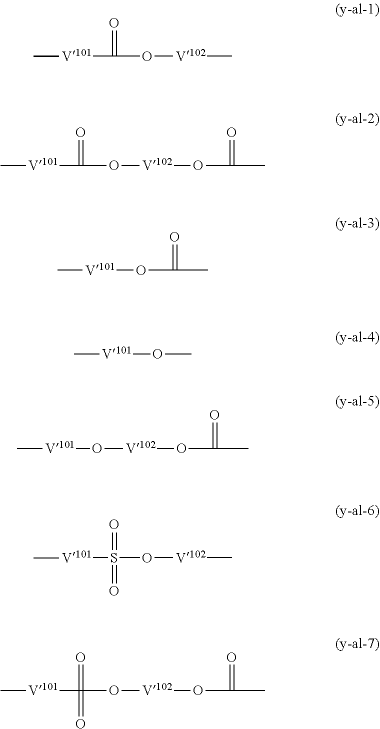

[In the formulae, V′¹⁰¹ represents an alkylene group having 1 to 5 carbon atoms or a single bond, and V′¹⁰² represents a divalent saturated hydrocarbon group having 1 to 30 carbon atoms or a single bond.]

As the divalent saturated hydrocarbon group as V′¹⁰², an alkylene group having 1 to 30 carbon atoms is preferable, an alkylene group having 1 to 10 carbon atoms is more preferable, and an alkylene group having 1 to 5 carbon atoms is still more preferable.

The alkylene group as V′¹⁰¹ and V′¹⁰² may be a linear alkylene group or a branched alkylene group, and a linear alkylene group is preferable.

Specific examples of the alkylene group as V′¹⁰¹ and V′¹⁰² include a methylene group [—CH₂—]; an alkylmethylene group such as —CH(CH₃)—, —CH(CH₂CH₃)—, —C(CH₃)₂—. —C(CH₃)(CH₂CH₃)—, —C(CH₃)(CH₂CH₂CH₃)—, or —C(CH₂CH₃)₂—; an ethylene group [—CH₂CH₂—]; an alkylethylene group such as —CH(CH₃)CH₂—, —CH(CH₃)CH(CH₃)—, —C(CH₃)₂CH₂—, or —CH(CH₂CH₀)CH₂—; a trimethylene group (n-propylene group) [—CH₂CH₂CH₂—]; an alkyltrimethylene group such as —CH(CH₃)CH₂CH₂— or —CH₂CH(CH₃)CH₂—; a tetramethylene group [—CH₂CH₂CH₂CH₂—]; an alkyltetramethylene group such as —CH(CH₃)CH₂CH₂CH₂— or —CH₂CH(CH₃)CH₂CH₂—; and a pentamethylene group [—CH₂CH₂CH₂CH₂CH₂-1.

Further, a part of methylene group in the alkylene group as V′¹⁰‘ and V’¹⁰ may be substituted with a divalent aliphatic cyclic group having 5 to 10 carbon atoms. The aliphatic cyclic group is preferably a divalent group in which one hydrogen atom has been removed from the cyclic aliphatic hydrocarbon group (a monocyclic aliphatic hydrocarbon group or a polycycic aliphatic hydrocarbon group) and is more preferably a cyclohexylene group, a 1,5-adamantylene group, or a 2.6-adamantylene group.

Y¹⁰¹ represents preferably a divalent linking group having an ester bond or a divalent linking group having an ether bond and more preferably an ester bond (—C(═O)—O—), an oxycarbonyl group (—O—C(═O)—), or a linking group represented by each of Formulae (y-a1-1) to (y-a1-5).

In Formula (b-1). V¹⁰¹ represents a single bond, an alkylene group, or a fluorinated alkylene group. It is preferable that the alkylene group and the fluorinated alkylene group as V¹⁰¹ have 1 to 4 carbon atoms. Examples of the fluorinated alkylene group as V¹⁰¹ include a group in which some or all hydrogen atoms in the alkylene group as V¹⁰¹ have been substituted with fluorine atoms. Among them, V¹⁰¹ is preferably a linear fluorinated alkylene group having 1 to 4 carbon atoms or a single bond.

In Formula (b-1), R¹⁰² represents a fluorine atom or a fluorinated alkyl group having 1 to 5 carbon atoms. R¹⁰² represents preferably a fluorine atom or a perfluoroalkyl group having 1 to 5 carbon atoms and more preferably a fluorine atom.

In a case where Y¹⁰¹ represents a single bond, specific example of the anion moiety represented by Formula (b-1) include a fluorinated alkylsulfonate anion such as a trifluoromethanesulfonate anion or a perfluorobutanesulfonate anion. Further, in a case where Y¹⁰¹ represents a divalent linking group having an oxygen atom, specific examples thereof include an anion represented by any of Formulae (an-1) to (an-3).

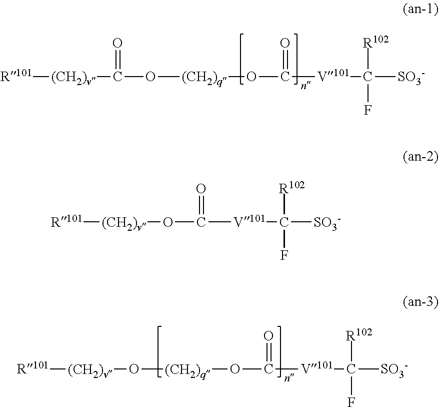

[in the formulae, R′¹⁰¹ represents an aliphatic cyclic group which may have a substituent, a —SO₂-containing cyclic group represented by each of General Formulae (b5-r-1) to (b5-r-4), a monovalent heterocyclic group represented by each of Chemical Formulae (r-hr-1) to (r-hr-6), an aryl group which may have a substituent, a condensed cyclic group represented by Chemical Formula (r-br-1) or (r-br-2), a chain-like alkyl group which may have a substituent, or an aromatic cyclic group which may have a substituent. R′¹⁰² represents an aliphatic cyclic group which may have a substituent, a condensed cyclic group represented by Formula (r-br-1) or (r-br-2), a lactone-containing cyclic group represented by each of General Formulae (a2-r-1) and (a2-r-3) to (a2-r-7), or a —SO₂-containing cyclic group represented by each of General Formulae (b5-r-1) to (b5-r-4). R″¹⁰³ represents an aromatic cyclic group which may have a substituent, an aliphatic cyclic group which may have a substituent, or a chain-like alkenyl group which may have a substituent. V′¹⁰¹ represents a single bond, an alkylene group having 1 to 4 carbon atoms, or a fluorinated alkylene group having 1 to 4 carbon atoms. R¹⁰² represents a fluorine atom or a fluorinated alkyl group having 1 to 5 carbon atoms. Each v″ independently represents an integer of 0 to 3, each q″ independently represents an integer of 0 to 20, and n″ represents 0 or 1.]

As the aliphatic cyclic group as R″¹⁰¹, R″¹⁰², and R″¹⁰³ which may have a substituent, the same groups as those for the cyclic aliphatic hydrocarbon group as R¹⁰¹ in Formula (b-1) are preferable. Examples of the substituent include the same groups as those for the substituent which may substitute the cyclic aliphatic hydrocarbon group as R¹⁰¹ in Formula (b-1).

As the aryl group which may have a substituent as R″¹⁰¹, an alkyl group having 1 to 5 carbon atoms or an aryl group having a substituent selected from the group consisting of a bromine atom and an iodine atom is preferable, an aryl group having a substituent selected from the group consisting of a bromine atom and an iodine atom is more preferable, and an aryl group having an iodine atom as a substituent is still more preferable.

As the aromatic cyclic group which may have a substituent as R″¹⁰¹ and R″¹⁰³, the same groups as those for the aromatic hydrocarbon group in the cyclic hydrocarbon group as R¹⁰¹ in Formula (b-1) are preferable. Examples of the substituent include the same groups as those for the substituent which may substitute the aromatic hydrocarbon group as R¹⁰¹ in Formula (b-1).

As the chain-like alkyl group as R″¹⁰¹ which may have a substituent, the same groups as those for the chain-like alkyl group as R¹¹⁰¹ in Formula (b-1) are preferable.

As the chain-like alkenyl group as R″(“which may have a substituent, the same groups as those for the chain-like alkenyl group as R¹¹¹ in Formula (b-1) are preferable.

Anions in Component (b-2)

In Formula (b-2), R¹⁰⁴ and R¹⁰⁵ each independently represent a cyclic group which may have a substituent, a chain-like alkyl group which may have a substituent, or a chain-like alkenyl group which may have a substituent, each of which has the same definition as that for R¹⁰¹ in Formula (b-1). Provided that, R¹⁰⁴ and R¹⁰⁵ may be bonded to each other to form a ring.

R¹⁰⁴ and R¹⁰⁵ represent preferably a chain-like alkyl group which may have a substituent and more preferably a linear or branched alkyl group or a linear or branched fluorinated alkyl group.

The chain-like alkyl group has preferably 1 to 10 carbon atoms, more preferably 1 to 7 carbon atoms, and still more preferably 1 to 3 carbon atoms. It is preferable that the number of carbon atoms in the chain-like alkyl group as R¹⁰⁴ and R¹⁰⁵ decreases within the range of the number of carbon atoms from the viewpoints of the satisfactory solubility in a solvent for a resist and the like.

In the chain-like alkyl group as R¹⁰⁴ and R¹⁰⁵, it is preferable that the number of hydrogen atoms substituted with fluorine atoms is as large as possible from the viewpoint that the acid strength increases and the transparency to high energy light or electron beams having a wavelength of 250 nm or less is improved. The proportion of fluorine atoms in the chain-like alkyl group, that is, the fluorination ratio is preferably in a range of 70% to 100% and more preferably in a range of 90% to 100%, and it is most preferable that the chain-like alkyl group is a perfluoroalkyl group in which all hydrogen atoms are substituted with fluorine atoms.

In Formula (b-2), V¹⁰² and V¹⁰³ each independently represent a single bond, an alkylene group, or a fluorinated alkylene group, and examples thereof include the same groups as those for V¹⁰¹ in Formula (b-1).

In Formula (b-2), L¹⁰¹ and L¹⁰² each independently represent a single bond or an oxygen atom.

Anions in Component (b-3)

In Formula (b-3), R¹⁰⁶ to R¹⁰⁸ each independently represent a cyclic group which may have a substituent, a chain-like alkyl group which may have a substituent, or a chain-like alkenyl group which may have a substituent, and examples thereof include the same groups as those for R¹⁰¹ in Formula (b-1).

In Formula (b-3), L¹⁰³ to L¹⁰⁵ each independently represent a single bond, —CO—, or —SO₂—.

Among the examples, as the anion moiety of the component (B), an anion in the component (b-1) is preferable.

### {Cation Moiety}

In Formulae (b-1), (b-2), and (b-3), M′^{m+} represents an m-valent onium cation. Among these, it is preferable that M′^{m+} represents a sulfonium cation or an iodonium cation, m represents an integer of 1 or greater.

Preferred examples of the cation moiety ((M′^{m+})_{1/m}) include organic cations each represented by General Formulae (ca-1) to (ca-3).

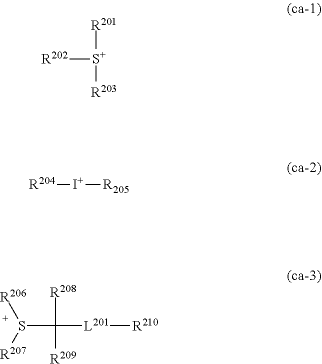

[In the formula, R²” to R²¹⁷ each independently represent an aryl group which may have a substituent, an alkyl group which may have a substituent, or an alkenyl group which may have a substituent. R²⁰¹ to R²⁰³, and R¹ and R²⁰⁷ may be bonded to each other to form a ring with the sulfur atoms in the formulae. R²⁸ and R²⁰⁹ each independently represent a hydrogen atom or an alkyl group having 1 to 5 carbon atoms. R²¹⁰ represents an aryl group which may have a substituent, an alkyl group which may have a substituent, an alkenyl group which may have a substituent, or a —SO₂-containing cyclic group which may have a substituent. L²⁰¹ represents —C(═O)— or —C(═O)—O—.]

In General Formulae (ca-1) to (ca-3), examples of the aryl group as R²⁰¹ to R²⁰⁷ include an unsubstituted aryl group having 6 to 20 carbon atoms. Among these, a phenyl group or a naphthyl group is preferable.

As the alkyl group as R²⁰¹ to R²⁰⁷, a chain-like or cyclic alkyl group having 1 to 30 carbon atoms is preferable.

It is preferable that the alkenyl group as R²⁰¹ to R²⁰⁷ has 2 to 10 carbon atoms.

Examples of the substituent that R²⁰¹ to R²¹⁷ and R²¹⁰ may have include an alkyl group, a halogen atom, a halogenated alkyl group, a carbonyl group, a cyano group, an amino group, an aryl group, and a group represented by each of General Formulae (ca-r-1) to (ca-r-8). Among these, from the viewpoint of enhancing the sensitivity, a halogen atom or a halogenated alkyl group is preferable, and a fluorine atom or a fluorinated alkyl group is more preferable.

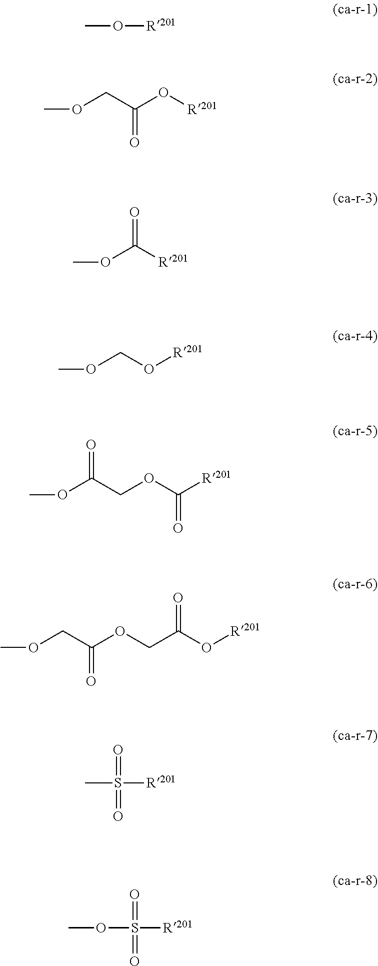

[In the formulae, R″²⁰¹'s each independently represent a hydrogen atom, a cyclic group which may have a substituent, a chain-like alkyl group which may have a substituent, or a chain-like alkenyl group which may have a substituent.]

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group with no aromaticity. Further, the aliphatic hydrocarbon group may be saturated or unsaturated. In general, it is preferable that the aliphatic hydrocarbon group is saturated.

The aromatic hydrocarbon group as R¹²¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Provided that, the number of carbon atoms in a substituent is not included in the number of carbon atoms.

Specific examples of the aromatic ring contained in the aromatic hydrocarbon group as R′²⁰¹ include benzene, fluorene, naphthalene, anthracene, phenanthrene, biphenyl, or an aromatic heterocyclic ring in which some carbon atoms constituting any of these aromatic rings are substituted with heteroatoms. Examples of the heteroatom in the aromatic heterocyclic rings include an oxygen atom, a sulfur atom, and a nitrogen atom.

Specific examples of the aromatic hydrocarbon group as R′²⁰¹ include a group in which one hydrogen atom has been removed from the aromatic ring (an aryl group such as a phenyl group or a naphthyl group), and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R′²⁰¹ include an aliphatic hydrocarbon group having a ring in the structure thereof.

Examples of the aliphatic hydrocarbon group having a ring in the structure thereof include an alicyclic hydrocarbon group (a group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The monocycloalkane has preferably 3 to 6 carbon atoms, and specific examples thereof include cyclopentane and cyclohexane. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring polycyclic skeleton such as adamantane, norbornane, isobornane, tricyclo[5.2.1.0^{2,6}]decane, or tetracyclododecane; and a polycycloalkane having a condensed ring polycyclic skeleton such as a cyclic group having a steroid skeleton are preferable as the polycycloalkane.

Among these examples, as the cyclic aliphatic hydrocarbon group as R′²⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is particularly preferable, and an adamantyl group is most preferable.

The linear or branched aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and particularly preferably 1 to 3 carbon atoms.

As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—. —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—. —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. As the alkyl group in the alkylalkylene group, a linear alkyl group having 1 to 5 carbon atoms is preferable.

Further, the cyclic hydrocarbon group as R¹²¹ may have a heteroatom such as a heterocyclic ring. Specific examples thereof include lactone-containing cyclic groups each represented by General Formulae (a2-r-1) to (a2-r-7), —SO₂-containing cyclic group each represented by General Formulae (b5-r-1) to (b5-r-4), and other heterocyclic groups each represented by Chemical Formulae (r-hr-1) to (r-hr-16).

Examples of the substituent for the cyclic group as R′²⁰¹ include an alkyl group, an alkoxy group, a halogen atom, a halogenated alkyl group, a hydroxyl group, a carbonyl group, and a nitro group.

As the alkyl group as the substituent, an alkyl group having 1 to 5 carbon atoms is preferable, and a methyl group, an ethyl group, a propyl group, an n-butyl group, or a tert-butyl group is most preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

As the halogen atom as a substituent, a fluorine atom is preferable.

Example of the above-described halogenated alkyl group as the substituent includes a group in which some or all hydrogen atoms in an alkyl group having 1 to 5 carbon atoms such as a methyl group, an ethyl group, a propyl group, an n-butyl group, or a tert-butyl group are substituted with the above-described halogen atoms.

The carbonyl group as the substituent is a group that substitutes a methylene group (—CH₂—) constituting the cyclic hydrocarbon group.

Chain-Like Alkyl Group which May have Substituent:

The chain-like alkyl group as R′²⁰¹ may be linear or branched.

The linear alkyl group has preferably 1 to 20 carbon atoms, more preferably 1 to 15 carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a I-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-Like Alkenyl Group which May have Substituent:

The chain-like alkenyl group as R′²⁰¹ may be linear or branched, and the number of carbon atoms thereof is preferably in a range of 2 to 10, more preferably in a range of 2 to 5, still more preferably in a range of 2 to 4, and particularly preferably 3. Examples of the linear alkenyl group include a vinyl group, a propenyl group (an allyl group), and a butynyl group. Examples of the branched alkenyl group include a 1-methylvinyl group, a 2-methylvinyl group, a 1-methylpropenyl group, and a 2-methylpropenyl group. Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R′²⁰¹ include an alkoxy group, a halogen atom, a halogenated alkyl group, a hydroxyl group, a carbonyl group, a nitro group, an amino group, and a cyclic group as R′²⁰¹.

Examples of the cyclic group which may have a substituent, the chain-like alkyl group which may have a substituent, and the chain-like alkenyl group which may have a substituent as R′²⁰¹ include those for the acid dissociable group represented by Formula (a1-r-2) which are the exemplary examples of the cyclic group which may have a substituent and the chain-like alkyl group which may have a substituent, in addition to those described above.

Among the examples, R′²⁰¹ represents preferably a cyclic group which may have a substituent and more preferably a cyclic hydrocarbon group which may have a substituent. More specific preferred examples thereof include a phenyl group, a naphthyl group, a group in which one or more hydrogen atoms have been removed from a polycycloalkane, a lactone-containing cyclic group represented by each of General Formulae (a2-r-1) to (a2-r-7), and a —SO₂-containing cyclic group represented by each of General Formulae (b5-r-1) to (b5-r-4).

In General Formulae (ca-1) to (ca-3), in a case where R²⁰¹ to R²⁰³ and R²⁰⁶ and R²⁰⁷ are bonded to each other to form a ring with a sulfur atom in the formula, these groups may be bonded to each other via a heteroatom such as a sulfur atom, an oxygen atom, or a nitrogen atom, or a functional group such as a carbonyl group, —SO—, —SO₂—, —SO₂—, —COO—, —CONH— or —N(RN)—(RN represents an alkyl group having 1 to 5 carbon atoms). As a ring to be formed, a ring containing the sulfur atom in the formula in the ring skeleton thereof is preferably a 3- to 10-membered ring and particularly preferably a 5- to 7-membered ring containing the sulfur atom. Specific examples of the ring to be formed include a thiophene ring, a thiazole ring, a benzothiophene ring, a dibenzothiophene ring, a 9H-thioxanthene ring, a thioxanthone ring, a thianthrene ring, a phenoxathiin ring, a tetrahydrothiophenium ring, and a tetrahydrothiopyranium ring.

R²⁰⁸ and R²⁰⁹ each independently represent a hydrogen atom or an alkyl group having 1 to 5 carbon atoms and preferably a hydrogen atom or an alkyl group having 1 to 3 carbon atoms. In a case where R²⁰⁸ and R²⁰⁹ represent an alkyl group, R²⁰⁸ and R²⁰⁹ may be bonded to each other to form a ring.

R²¹⁰ represents an aryl group which may have a substituent, an alkyl group which may have a substituent, an alkenyl group which may have a substituent, or a —SO₂— containing cyclic group which may have a substituent.

Examples of the aryl group as R²¹⁰ include an unsubstituted aryl group having 6 to 20 carbon atoms. Among these, a phenyl group or a naphthyl group is preferable.

As the alkyl group as R²¹”, a chain-like or cyclic alkyl group having 1 to 30 carbon atoms is preferable.

It is preferable that the alkenyl group as R²¹⁰ has 2 to 10 carbon atoms. The —SO₂-containing cyclic group which may have a substituent as R²¹⁰ is preferably “—SO₂-containing polycyclic group” and more preferably a group represented by General Formula (b5-r-1).

Specific suitable examples of cations represented by Formula (ca-1) include cations each represented by Chemical Formulae (ca-1-1) to (ca-1-76).

From the viewpoint of enhancing the sensitivity, as the suitable cation represented by Formula (ca-1), a cation having a fluorine atom or a fluorinated alkyl group as a substituent is preferable, and a cation selected from the group consisting of cations each represented by Chemical Formulae (ca-1-69) to (ca-1-75) is particularly preferable.

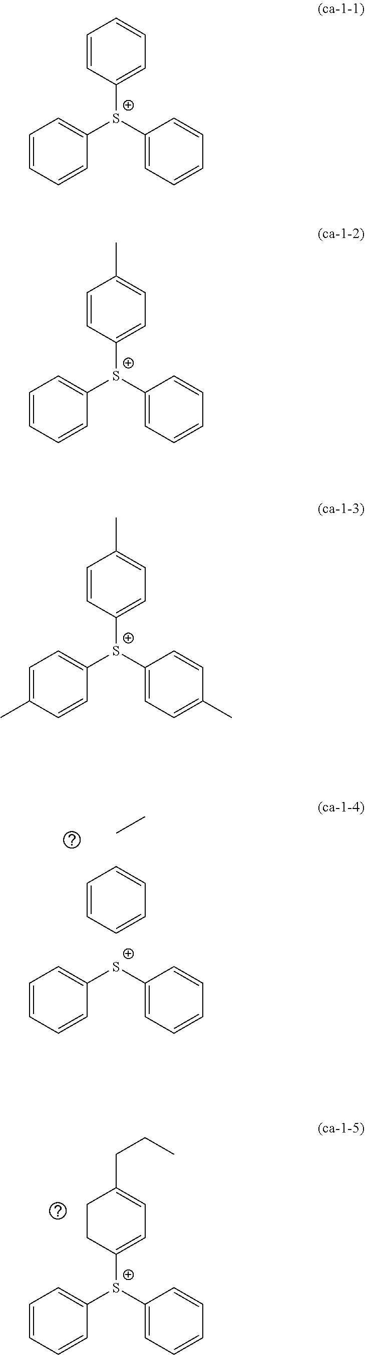

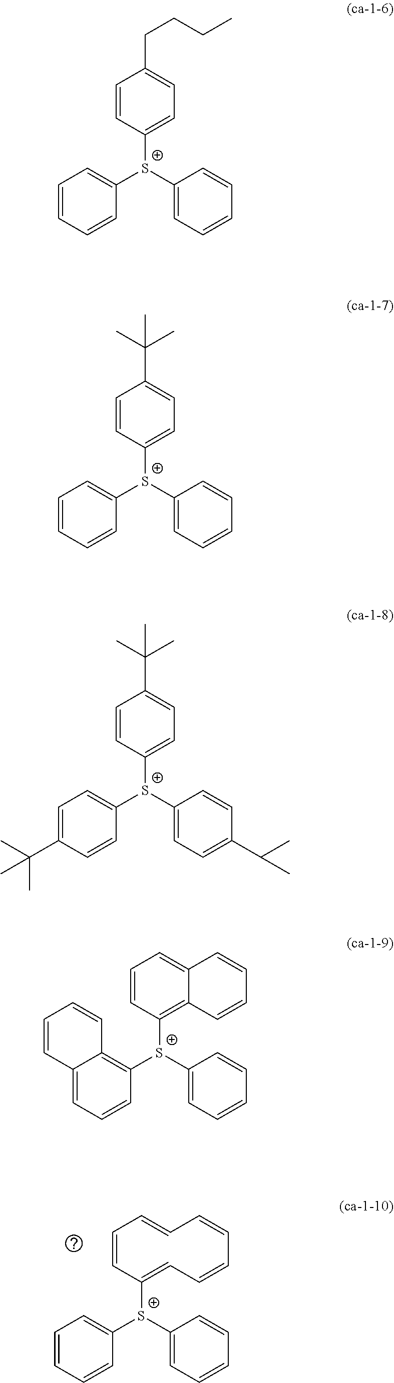 [SMILES: CCCCc1ccc([S+](c2ccccc2)c2ccccc2)cc1 ; CC(C)(C)c1ccc([S+](c2ccccc2)c2ccccc2)cc1 ; CC(C)c1ccc([S+](c2ccc(C(C)(C)C)cc2)c2ccc(C(C)(C)C)cc2)cc1 ; c1ccc([S+](c2cccc3ccccc23)c2cccc3ccccc23)cc1 ; c1ccccc([S+](c2ccccc2)c2ccccc2)cccc1]

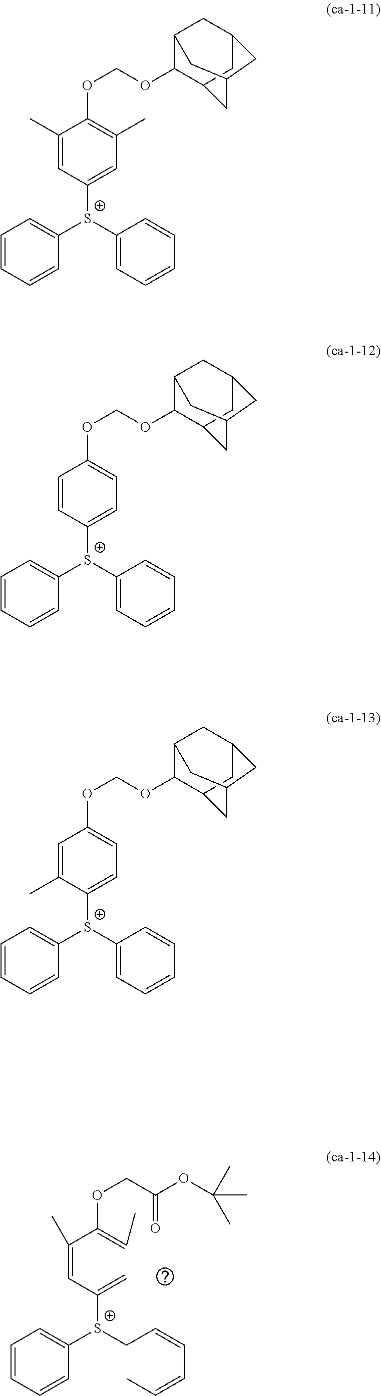 [SMILES: Cc1cc([S+](c2ccccc2)c2ccccc2)cc(C)c1OCOC1C2CC3CC(C2)CC1C3 ; c1ccc([S+](c2ccccc2)c2ccc(OCOC3C4CC5CC(C4)CC3C5)cc2)cc1 ; Cc1cc(OCOC2C3CC4CC(C3)CC2C4)ccc1[S+](c1ccccc1)c1ccccc1 ; C=C(/C=C(C)\C(=C\C)OCC(=O)OC(C)(C)C)[S+](C/C=C\C=C/C)c1ccccc1]

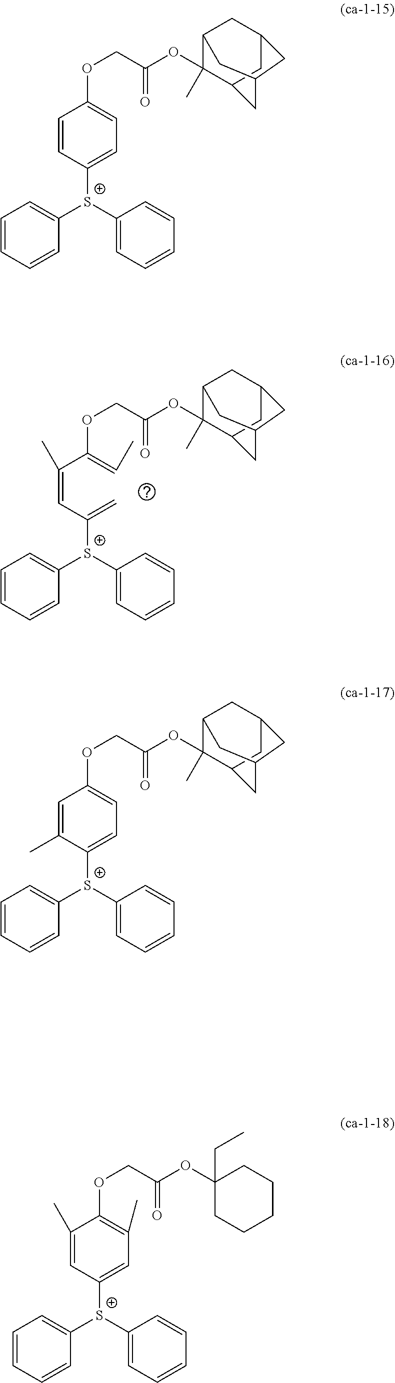 [SMILES: CC1(OC(=O)COc2ccc([S+](c3ccccc3)c3ccccc3)cc2)C2CC3CC(C2)CC1C3 ; C=C(/C=C(C)\C(=C\C)OCC(=O)OC1(C)C2CC3CC(C2)CC1C3)[S+](c1ccccc1)c1ccccc1 ; Cc1cc(OCC(=O)OC2(C)C3CC4CC(C3)CC2C4)ccc1[S+](c1ccccc1)c1ccccc1 ; CCC1(OC(=O)COc2c(C)cc([S+](c3ccccc3)c3ccccc3)cc2C)CCCCC1]

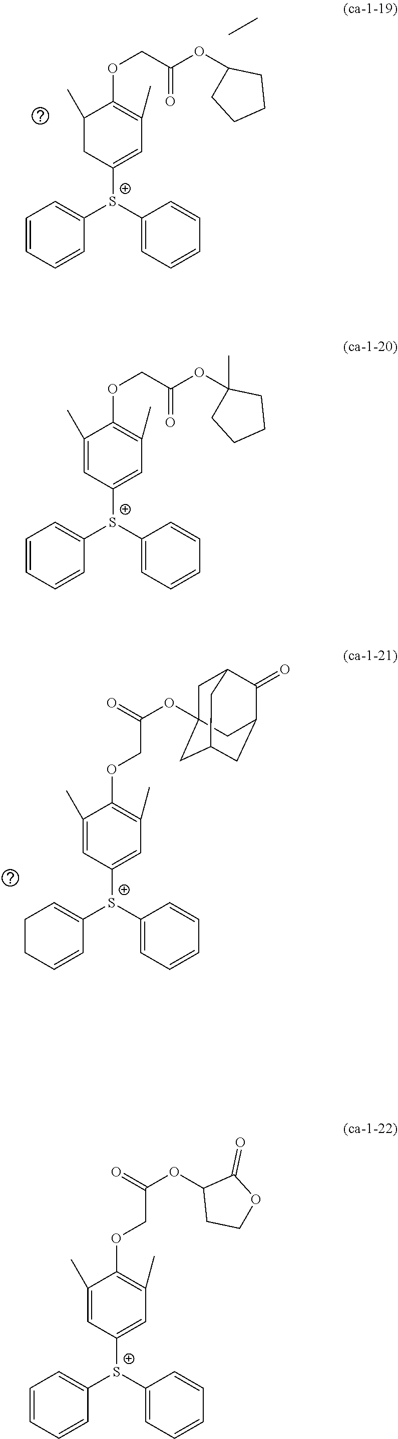

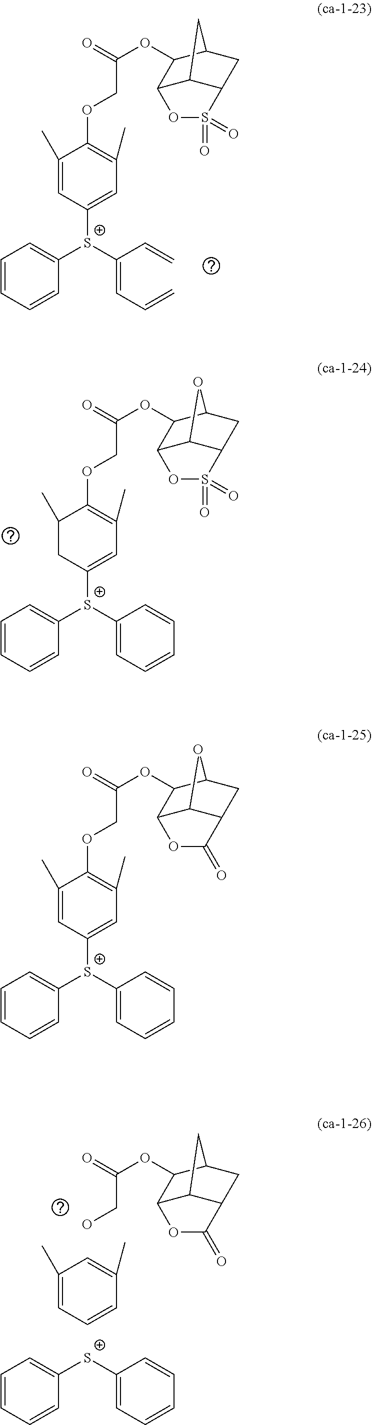

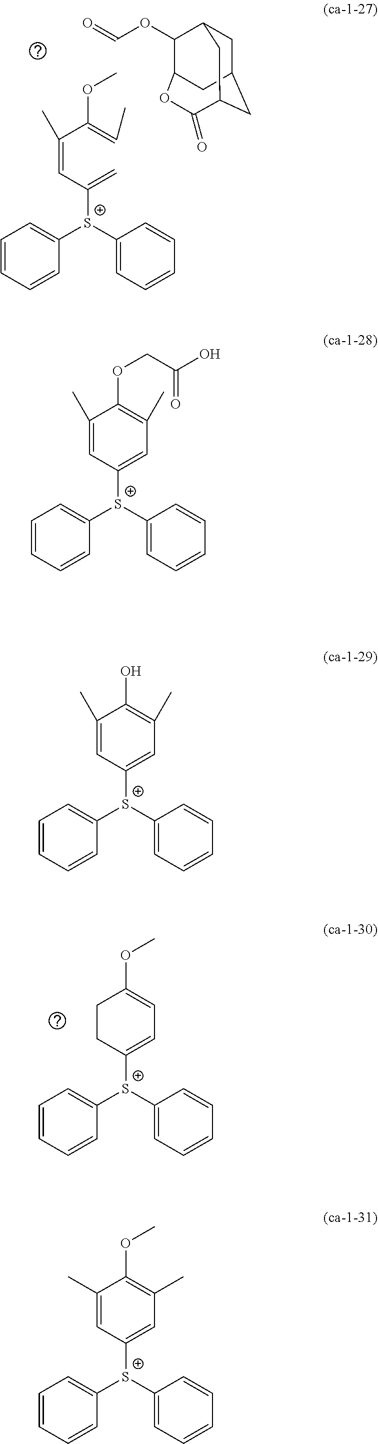 [SMILES: C=C(/C=C(C)\C(=C\C)OC)[S+](c1ccccc1)c1ccccc1 ; O=COC1C2CC3CC(C2)C(=O)OC1C3 ; Cc1cc([S+](c2ccccc2)c2ccccc2)cc(C)c1OCC(=O)O ; Cc1cc([S+](c2ccccc2)c2ccccc2)cc(C)c1O ; COC1=CC=C([S+](c2ccccc2)c2ccccc2)CC1 ; COc1c(C)cc([S+](c2ccccc2)c2ccccc2)cc1C]

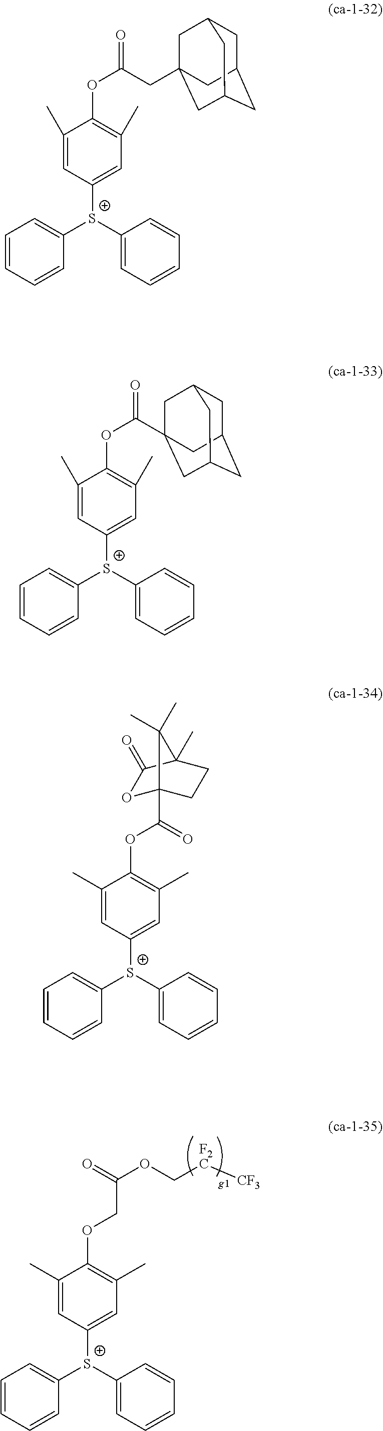

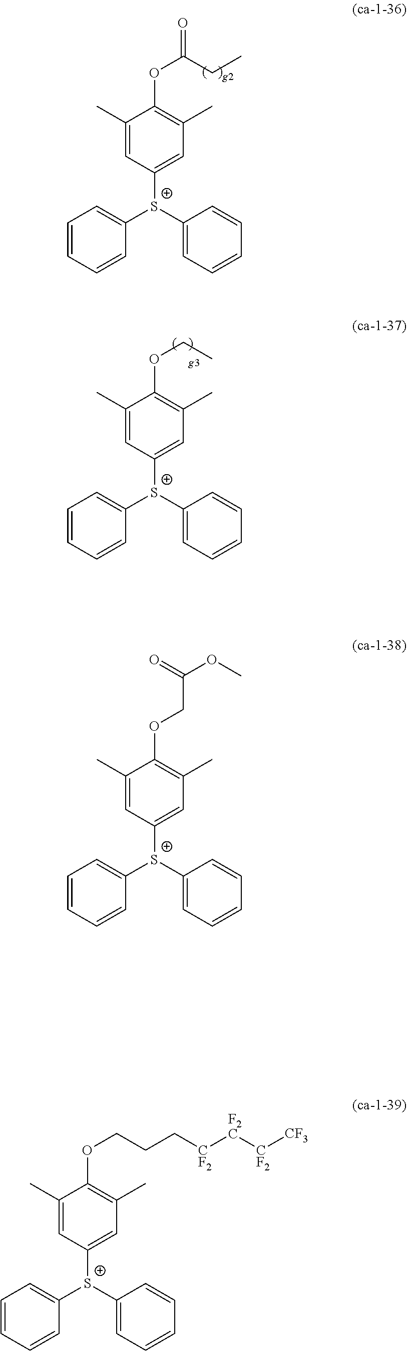

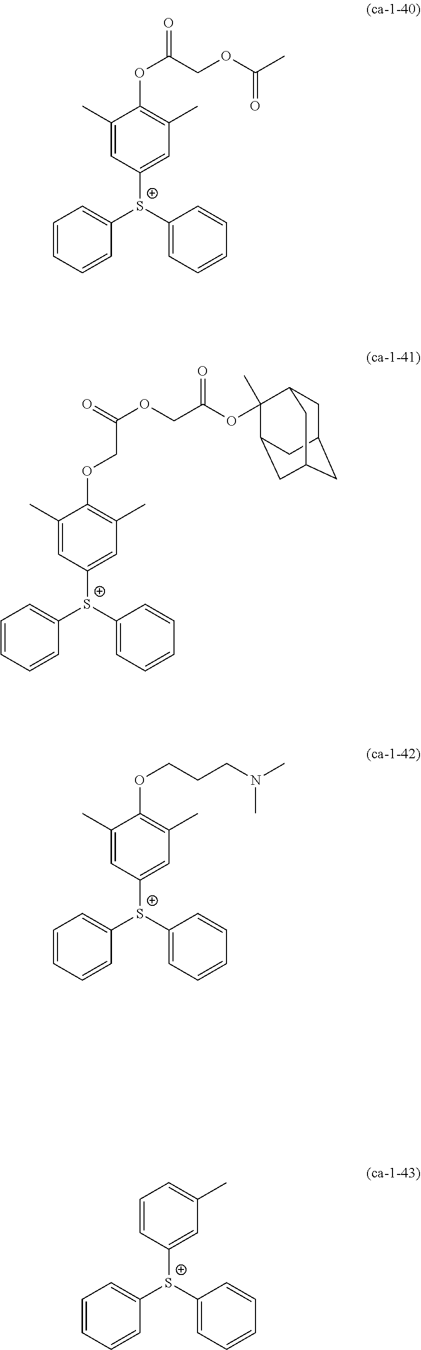 [SMILES: CC(=O)OCC(=O)Oc1c(C)cc([S+](c2ccccc2)c2ccccc2)cc1C ; Cc1cc([S+](c2ccccc2)c2ccccc2)cc(C)c1OCC(=O)OCC(=O)OC1(C)C2CC3CC(C2)CC1C3 ; Cc1cc([S+](c2ccccc2)c2ccccc2)cc(C)c1OCCCN(C)C ; Cc1cccc([S+](c2ccccc2)c2ccccc2)c1]

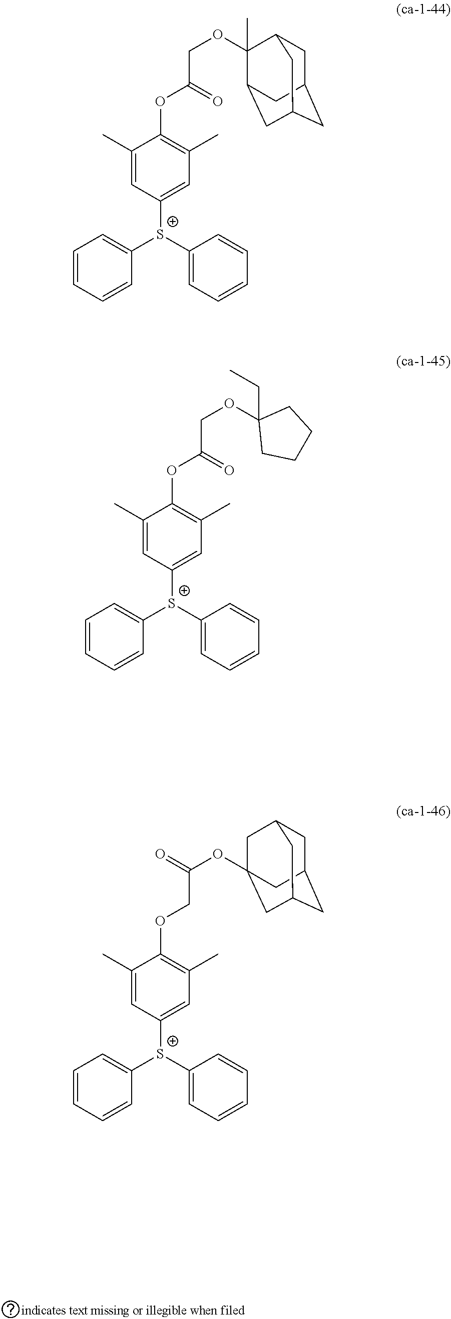 [SMILES: Cc1cc([S+](c2ccccc2)c2ccccc2)cc(C)c1OC(=O)COC1(C)C2CC3CC(C2)CC1C3 ; CCC1(OCC(=O)Oc2c(C)cc([S+](c3ccccc3)c3ccccc3)cc2C)CCCC1 ; Cc1cc([S+](c2ccccc2)c2ccccc2)cc(C)c1OCC(=O)OC12CC3CC(CC(C3)C1)C2]

[In the formulae, g1, g2, and g3 represent a repeating number, g1 represents an integer of 1 to 5, g2 represents an integer of 0 to 20, and g3 represents an integer of 0 to 20.]

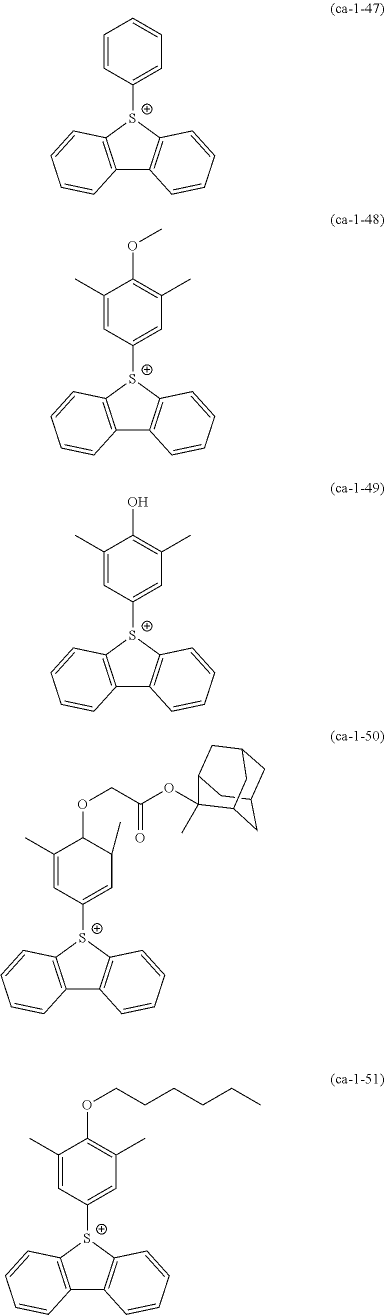 [SMILES: c1ccc(-[s+]2c3ccccc3c3ccccc32)cc1 ; COc1c(C)cc(-[s+]2c3ccccc3c3ccccc32)cc1C ; Cc1cc(-[s+]2c3ccccc3c3ccccc32)cc(C)c1O ; CC1=CC([s+]2c3ccccc3c3ccccc32)=CC(C)C1OCC(=O)OC1(C)C2CC3CC(C2)CC1C3 ; CCCCCCOc1c(C)cc(-[s+]2c3ccccc3c3ccccc32)cc1C]

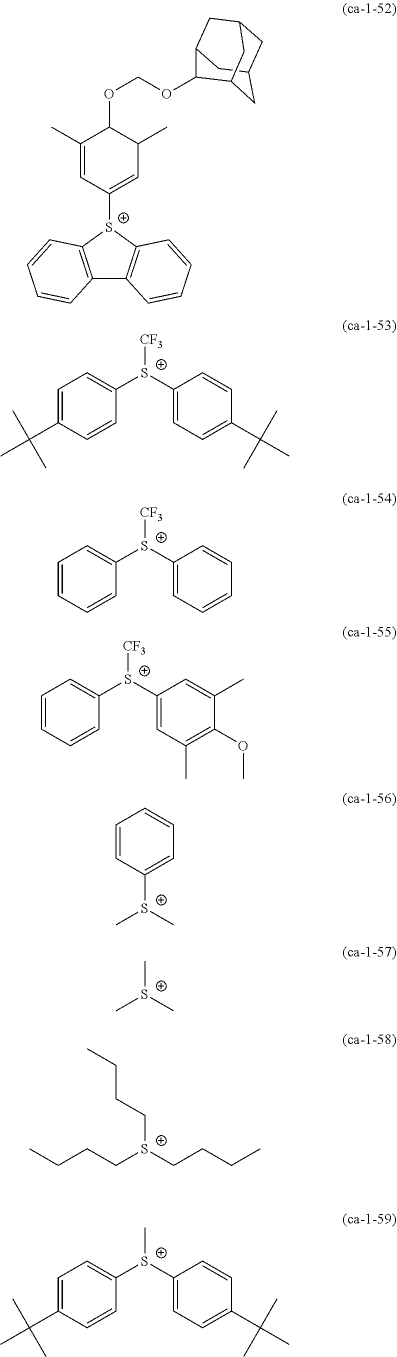

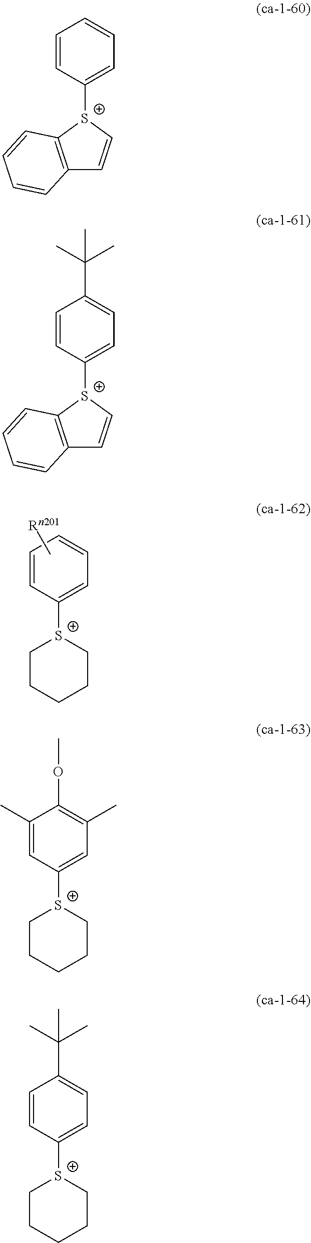

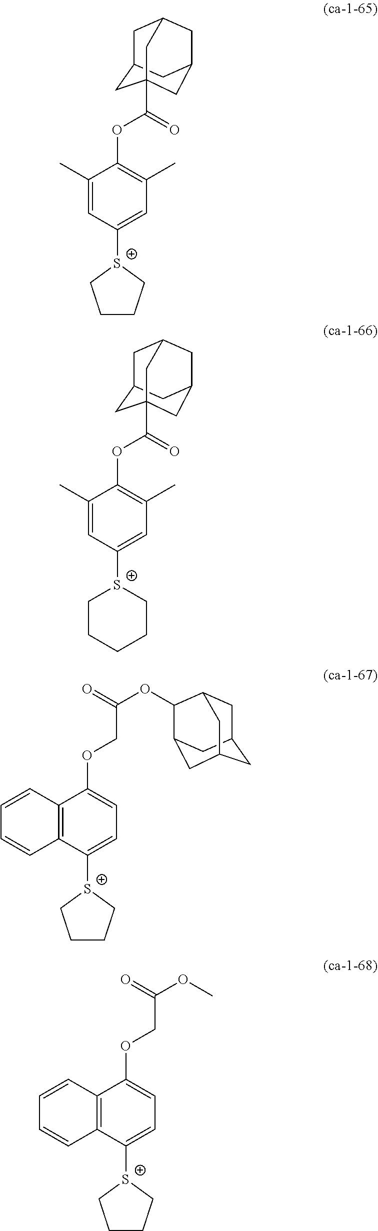 [SMILES: Cc1cc([S+]2CCCC2)cc(C)c1OC(=O)C12CC3CC(CC(C3)C1)C2 ; Cc1cc([S+]2CCCCC2)cc(C)c1OC(=O)C12CC3CC(CC(C3)C1)C2 ; O=C(COc1ccc([S+]2CCCC2)c2ccccc12)OC1C2CC3CC(C2)CC1C3 ; COC(=O)COc1ccc([S+]2CCCC2)c2ccccc12]

[In the formula, R″²⁰¹ represents a hydrogen atom or a substituent, and the substituent is the same as the substituent which may be contained in Ra²⁰¹ to R²⁰⁷ and R²¹⁰.]

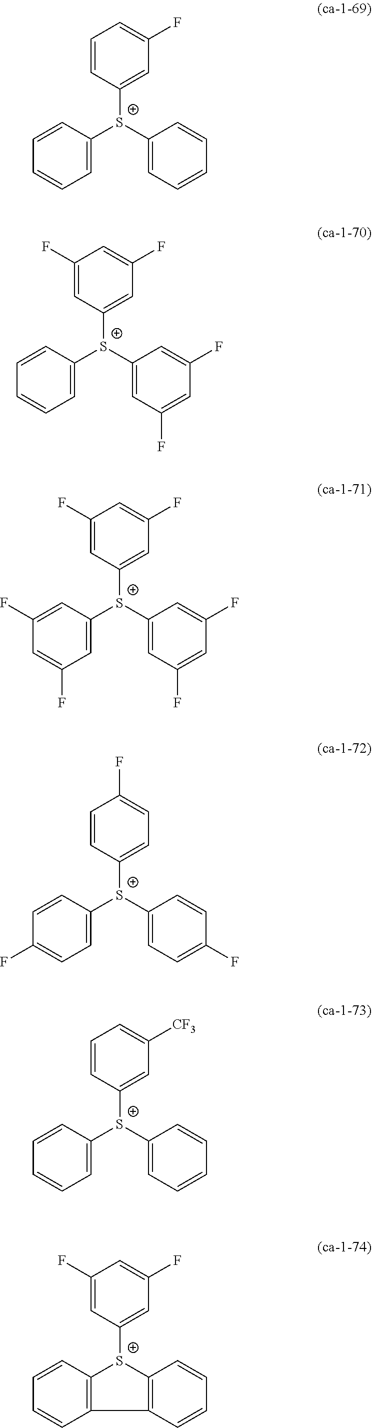 [SMILES: Fc1cccc([S+](c2ccccc2)c2ccccc2)c1 ; Fc1cc(F)cc([S+](c2ccccc2)c2cc(F)cc(F)c2)c1 ; Fc1cc(F)cc([S+](c2cc(F)cc(F)c2)c2cc(F)cc(F)c2)c1 ; Fc1ccc([S+](c2ccc(F)cc2)c2ccc(F)cc2)cc1 ; FC(F)(F)c1cccc([S+](c2ccccc2)c2ccccc2)c1 ; Fc1cc(F)cc(-[s+]2c3ccccc3c3ccccc32)c1]

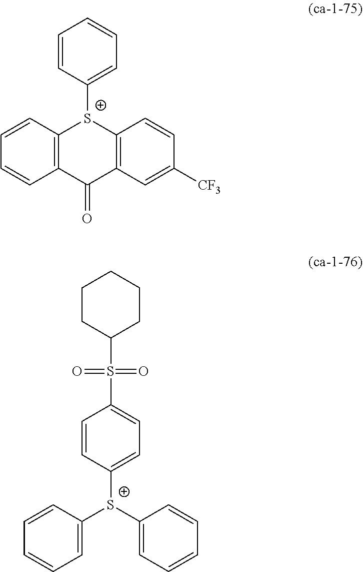 [SMILES: O=c1c2ccccc2[s+](-c2ccccc2)c2ccc(C(F)(F)F)cc12 ; O=S(=O)(c1ccc([S+](c2ccccc2)c2ccccc2)cc1)C1CCCCC1]

Specific examples of suitable cations represented by Formula (ca-2) include a diphenyliodoniumi cation and a bis(4-tert-butylphenyl)iodonium cation.

Specific examples of suitable cations represented by Formula (ca-3) include cations each represented by Formulae (ca-3-1) to (ca-3-6).

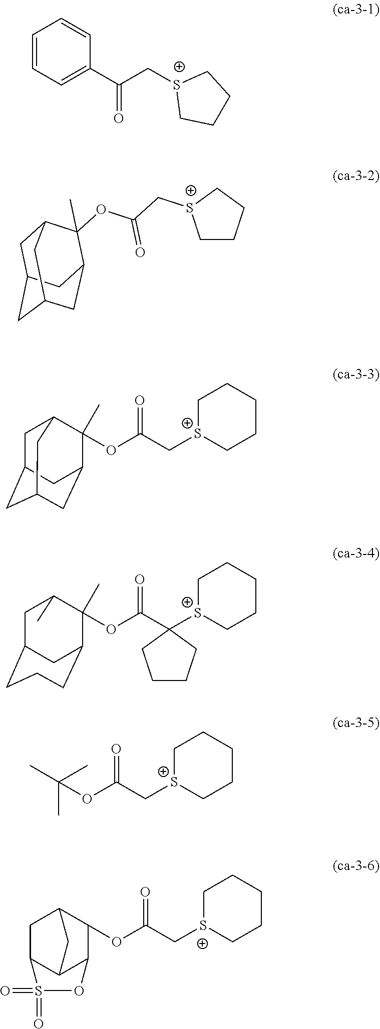 [SMILES: O=C(C[S+]1CCCC1)c1ccccc1 ; CC1(OC(=O)C[S+]2CCCC2)C2CC3CC(C2)CC1C3 ; CC1(OC(=O)C[S+]2CCCCC2)C2CC3CC(C2)CC1C3 ; CC1CC2CCCC(C2)C1(C)OC(=O)C1([S+]2CCCCC2)CCCC1 ; CC(C)(C)OC(=O)C[S+]1CCCCC1 ; O=C(C[S+]1CCCCC1)OC1C2CC3C1OS(=O)(=O)C3C2]

Among the examples, an organic cation represented by General Formula (ca-1) is more preferable as the cation moiety ((M′^{m+})_{1/m}).

Specific suitable examples of the component (B) in the resist composition according to the present embodiment are shown below.

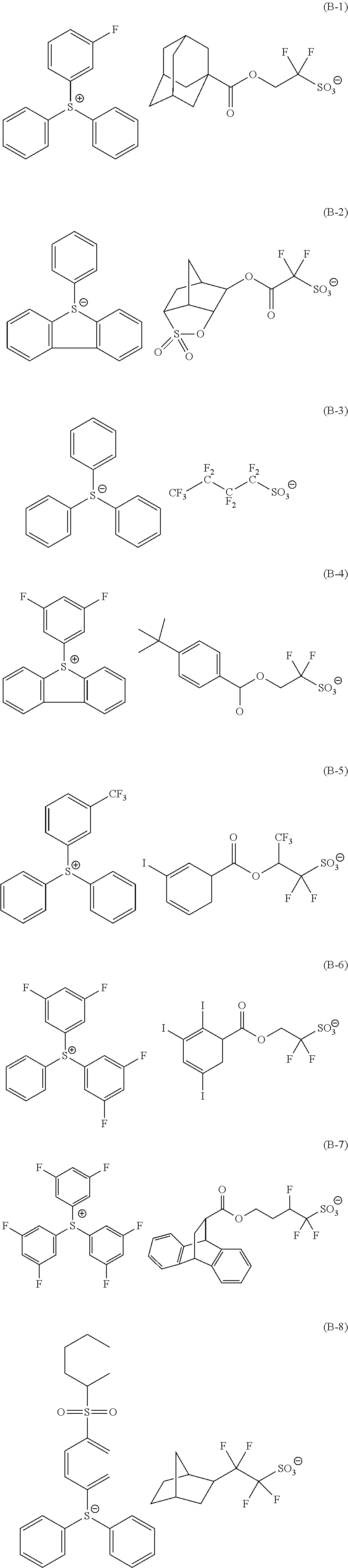

In the resist composition according to the present embodiment, as the (B) component, it is preferable to use at least one selected from the group consisting of the compounds represented by each of Chemical Formulae (B-1) to (B-8). Among these, from the viewpoint of improving all of sensitivity, roughness characteristics, and etching resistance, it is more preferable to use at least one selected from the group consisting of the compounds represented by each of Chemical Formulae (B-4) to (B-6), and it is still more preferable to use at least one selected from the group consisting of the compounds represented by each of Chemical Formulae (B-5) and (B-6).

In the resist composition according to the present embodiment, the component (B) may be used alone or in combination of two or more kinds thereof.

In a case where the resist composition contains the component (B), the content of the component (B) in the resist composition is preferably less than 60 parts by mass, more preferably in a range of 10 to 50 parts by mass, and still more preferably in a range of 20 to 50 parts by mass with respect to 100 parts by mass of the component (A1).

In a case where the content of the component (B) is set to be in the above-described preferable ranges, pattern formation can be sufficiently carried out. Further, it is preferable that each component of the resist composition is dissolved in an organic solvent from the viewpoint that a uniform solution is easily obtained and the storage stability of the resist composition is improved.

### <<Base Component (D)>>

It is preferable that the resist composition according to the present embodiment further contains a base component (hereinafter, also referred to as “component (D)”) that traps (that is, controls the acid diffusion) an acid that is generated upon light exposure, in addition to the component (A1) or the component (A1) and the component (B). Such a component (D) acts as a quencher (an acid diffusion control agent) that traps acid that is generated in the resist composition upon exposure.

Examples of the component (D) include a photodecomposable base (D1) having an acid diffusion controllability (hereinafter, referred to as “component (D1)”) which is lost by the decomposition upon light exposure and a nitrogen-containing organic compound (D2) (hereinafter, referred to as “component (D2)”) which does not correspond to the component (D1). Among these, the photodecomposable base (the component (D1)) is preferable since it is easy to enhance the roughness characteristic. Further, in a case where the component (D1) is contained, both the characteristics of enhancing the sensitivity and suppressing the occurrence of coating defects are likely to be enhanced.

### In Regard to Component (D1)

In a case where a resist composition containing the component (D1) is obtained, the contrast between an exposed portion and an unexposed portion of the resist film can be further improved in a case of forming a resist pattern.

The component (D) is not particularly limited as long as the component is decomposed upon light exposure and loses an acid diffusion controllability, and one or more compounds selected from the group consisting of a compound represented by General Formula (d1-1) (hereinafter, referred to as “component (d1-1)”), a compound represented by General Formula (d1-2) (hereinafter, referred to as “component (d1-2)”), and a compound represented by General Formula (d1-3) (hereinafter, referred to as “component (d1-3)”) are preferable.

Since the components (d1-1) to (d1-3) are decomposed and lose the acid diffusion controllability (basicity), the components (d1-1) to (d1-3) do not function as a quencher at the exposed portion of the resist film, but function as a quencher at the unexposed portion of the resist film.

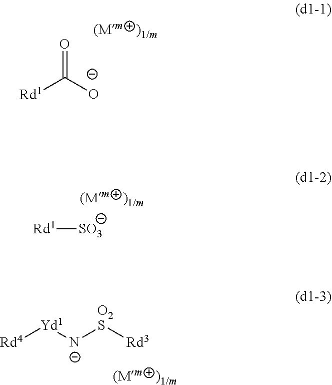

[In the formulae, Rd¹ to Rd⁴ represent a cyclic group which may have a substituent, a chain-like alkyl group which may have a substituent, or a chain-like alkenyl group which may have a substituent. Provided that, no fluorine atom is bonded to the carbon atom adjacent to the S atom in Rd² of Formula (d1-2). Yd¹ represents a divalent linking group or a single bond, in represents an integer of 1 or greater, and M′^{m+}'s each independently represent an m-valent organic cation.]

{Component (d1-1)}

### Anion Moiety

In Formula (d1-1), Rd¹ represents a cyclic group which may have a substituent, a chain-like alkyl group which may have a substituent, or a chain-like alkenyl group which may have a substituent, and examples thereof include the same groups as those for R′²⁰¹

Among these, Rd¹ represents preferably an aromatic hydrocarbon group which may have a substituent, an aliphatic cyclic group which may have a substituent, or a chain-like alkyl group which may have a substituent and more preferably an aromatic hydrocarbon group which may have a substituent.

Examples of the substituent that these groups may have include a hydroxyl group, an oxo group, an alkyl group, an aryl group, a fluorine atom, a bromine atom, an iodine atom, a fluorinated alkyl group, lactone-containing cyclic groups each represented by General Formulae (a2-r-1) to (a2-r-7), an ether bond, an ester bond, and a combination thereof. Among these, iodine atom is preferable.

In a case where an ether bond or an ester bond is included as the substituent, the substituent may be bonded through an alkylene group, and a linking group represented by each of Formulae (y-a1-1) to (y-a1-5) is preferable as the substituent. Further, in a case where the aromatic hydrocarbon group, the aliphatic cyclic group, or the chain-like alkyl group as Rd¹ contain a linking group represented by each of General Formulae (y-a1-1) to (y-a1-7) as a substituent, V′¹⁰¹ in General Formulae (y-a1-1) to (y-a1-7) is bonded to the carbon atom constituting the aromatic hydrocarbon group, the aliphatic cyclic group, or the chain-like alkyl group as Rd¹ in Formula (d1-1), in General Formulae (y-a1-1) to (y-a1-7).

Suitable examples of the aromatic hydrocarbon group include a phenyl group, a naphthyl group, and a polycyclic structure having a bicyclooctane skeleton (for example, a polycyclic structure formed of a bicyclooctane skeleton and a ring structure other than the bicyclooctane skeleton).

As the aliphatic cyclic group, a group in which one or more hydrogen atoms have been removed from a polycycloalkane such as adamantane, norbornane, isobornane, tricyclo[5.2.1.0^{2,6}]decane or tetracyclododecane is more preferable.

It is preferable that the chain-like alkyl group has 1 to 10 carbon atoms, and specific examples thereof include a linear alkyl group such as a methyl group, an ethyl group, a propyl group, a butyl group, a pentyl group, a hexyl group, a heptyl group, an octyl group, a nonyl group, or a decyl group; and a branched alkyl group such as a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, or a 4-methylpentyl group.

In a case where the chain-like alkyl group is a fluorinated alkyl group having a fluorine atom or a fluorinated alkyl group as a substituent, the fluorinated alkyl group has preferably 1 to 11 carbon atoms, more preferably 1 to 8 carbon atoms, and still more preferably 1 to 4 carbon atoms. The fluorinated alkyl group may have an atom other than a fluorine atom. Examples of the atom other than a fluorine atom include an oxygen atom, a sulfur atom, and a nitrogen atom.

Specific preferred examples of the anion moiety in the component (d1-1) are described below.

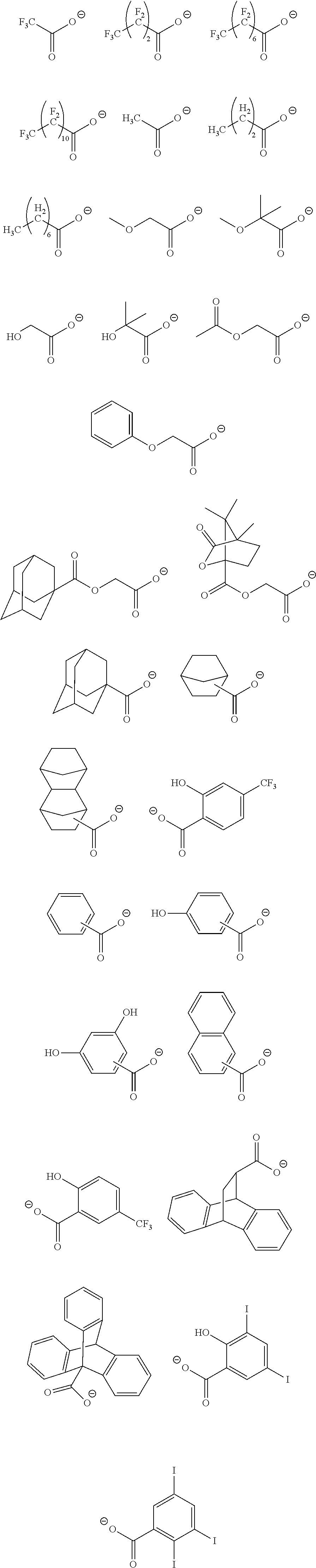

### Cation Moiety

In Formula (d1-1), M′^{m+} represents an m-valent organic cation.

Suitable examples of the organic cation as M^{m+} include those for the cations each represented by General Formulae (ca-1) to (ca-3). Among these, a cation represented by General Formula (ca-1) is more preferable, and a cation represented by each of Chemical Formulae (ca-1-1) to (ca-1-76) is still more preferable. From the viewpoint of enhancing the sensitivity, a cation having a fluorine atom or a fluorinated alkyl group as a substituent is preferable, and a cation selected from the group consisting of cations each represented by Chemical Formulae (ca-1-69) to (ca-1-75) is particularly preferable.

The component (d1-1) may be used alone or in combination of two or more kinds thereof.

(Component (d1-2))

### Anion Moiety

In Formula (d1-2), Rd² represents a cyclic group which may have a substituent, a chain-like alkyl group which may have a substituent, or a chain-like alkenyl group which may have a substituent, and examples thereof include the same groups as those for R′²⁰¹.

Provided that, no fluorine atom is bonded to the carbon atom adjacent to the S atom in Rd² (the carbon atom is not substituted with fluorine). In this manner, the anion of the component (d1-2) is an appropriately weak acid anion, thereby improving the quenching ability of the component (D).

Rd² represents preferably a chain-like alkyl group which may have a substituent or an aliphatic cyclic group which may have a substituent and more preferably an aliphatic cyclic group which may have a substituent.

The chain-like alkyl group preferably has 1 to 10 carbon atoms and more preferably 3 to 10 carbon atoms.

The aliphatic cyclic group is more preferably a group (which may have a substituent) obtained by removing one or more hydrogen atoms from adamantane, norbornane, isobornane, tricyclo[5.2.1.0^{2,6}]decane, tetracyclododecane, or the like; and a group obtained by removing one or more hydrogen atoms from camphor.

The hydrocarbon group as Rd² may have a substituent, and examples of the substituent include the same groups as those for the substituent that the hydrocarbon group (such as an aromatic hydrocarbon group, an aliphatic cyclic group, or a chain-like alkyl group) as Rd¹ in Formula (d1-1) may have.

Specific preferred examples of the anion moiety in the component (d1-2) are described below.

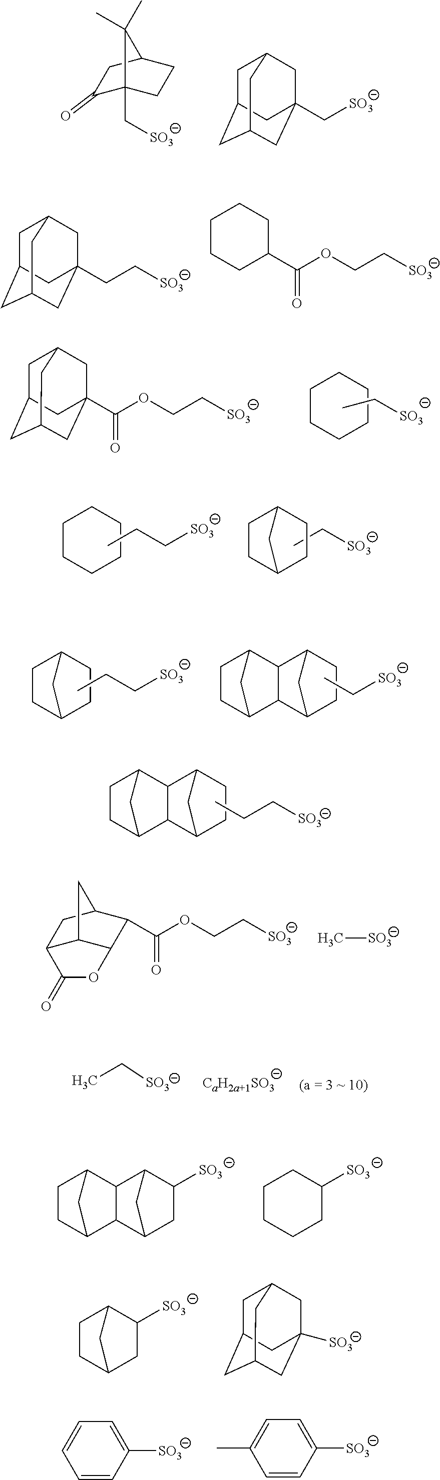

### Cation Moiety

In Formula (d1-2), M^{m+} represents an m-valent organic cation and has the same definition as that for M^{m+} in Formula (d1-1).

The component (d1-2) may be used alone or in combination of two or more kinds thereof.

(Component (d1-3)}

### Anion Moiety

In Formula (d1-3), Rd³ represents a cyclic group which may have a substituent, a chain-like alkyl group which may have a substituent, or a chain-like alkenyl group which may have a substituent, and examples thereof include the same groups as those for R′²⁰¹. Among these, a cyclic group having a fluorine atom, a chain-like alkyl group, or a chain-like alkenyl group is preferable. Among these, a fluorinated alkyl group is preferable, and the same groups as those for the fluorinated alkyl group represented by Rd¹ are more preferable.

In Formula (d1-3), Rd⁴ represents a cyclic group which may have a substituent, a chain-like alkyl group which may have a substituent, or a chain-like alkenyl group which may have a substituent, and examples thereof include the same groups as those for R′²⁰¹.

Among these, an alkyl group which may have a substituent, an alkoxy group which may have a substituent, an alkenyl group which may have a substituent, or a cyclic group which may have a substituent is preferable.

It is preferable that the alkyl group as Rd⁴ is a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. Some hydrogen atoms in the alkyl group as Rd⁴ may be substituted with a hydroxyl group, a cyano group, or the like.

It is preferable that the alkoxy group as Rd⁴ is an alkoxy group having 1 to 5 carbon atoms, and specific examples of the alkoxy group having 1 to 5 carbon atoms include a methoxy group, an ethoxy group, an n-propoxy group, an iso-propoxy group, an n-butoxy group, and a tert-butoxy group. Among these, a methoxy group and an ethoxy group are preferable.

Examples of the alkenyl group as Rd⁴ include the same groups as those for the alkenyl group as R′²⁰¹. Among these, a vinyl group, a propenyl group (an allyl group), a 1-methylpropenyl group, and a 2-methylpropenyl group are preferable. These groups may have an alkyl group having 1 to 5 carbon atoms or a halogenated alkyl group having 1 to 5 carbon atoms as a substituent.

Examples of the cyclic group as Rd⁴ include the smite groups as those for the cyclic group as R′²⁰¹. Among these, an alicyclic group in which one or more hydrogen atoms have been removed from a cycloalkane such as cyclopentane, cyclohexane, adamantane, norbornane, isobornane, tricyclo[5.2.1.0^{2,6}]decane or tetracyclododecane or an aromatic group such as a phenyl group or a naphthyl group is preferable. In a case where Rd⁴ represents an alicyclic group, the resist composition is satisfactorily dissolved in an organic solvent so that the lithography characteristics are enhanced. Further, in a case where Rd⁴ represents an aromatic group, the resist composition has excellent light absorption efficiency in lithography using EUV or the like as an exposure light source, and thus the sensitivity and lithography characteristics are enhanced.

In Formula (d1-3), Yd¹ represents a single bond or a divalent linking group.

The divalent linking group as Yd¹ is not particularly limited, and examples thereof include a divalent hydrocarbon group (an aliphatic hydrocarbon group or an aromatic hydrocarbon group) which may have a substituent and a divalent linking group having a heteroatom. The divalent hydrocarbon group and the divalent linking group each have the same definition as that for the divalent hydrocarbon group which may have a substituent and the divalent linking group having a heteroatom, described as groups other than the polymerizable group represented by W⁰¹.

It is preferable that Yd¹ represents a carbonyl group, an ester bond, an amide bond, an alkylene group, or a combination thereof. As the alkylene group, a linear or branched alkylene group is more preferable, and a methylene group or an ethylene group is still more preferable.

Specific preferred examples of the anion moiety in the component (d1-3) are described below.

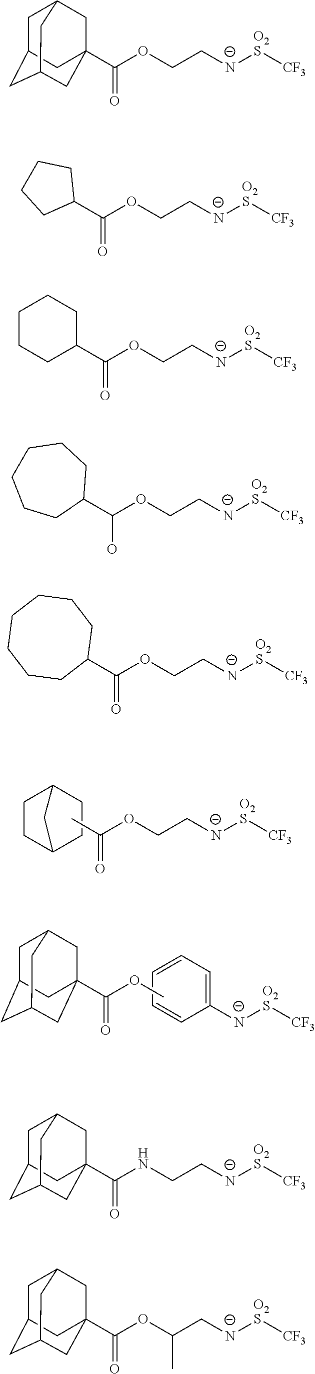

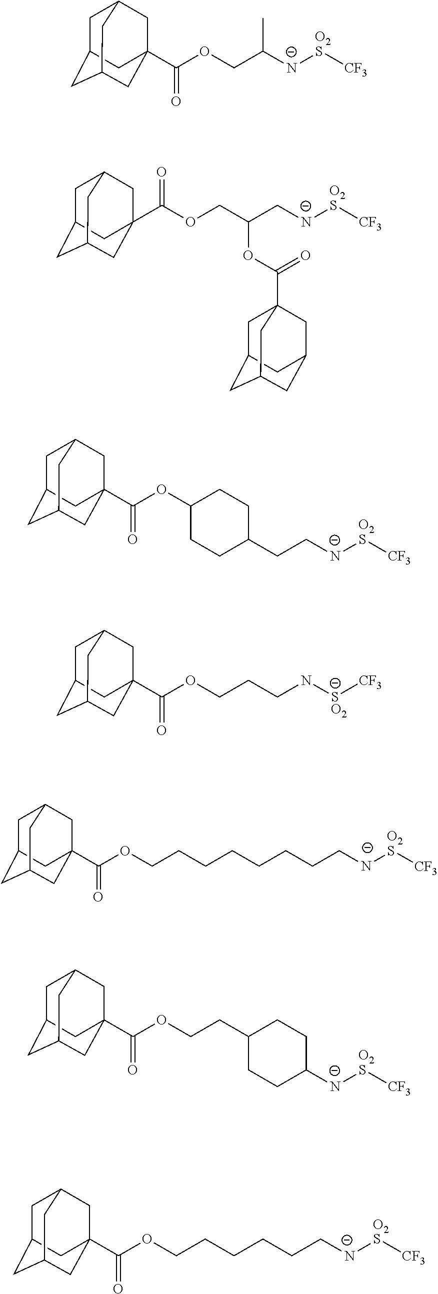

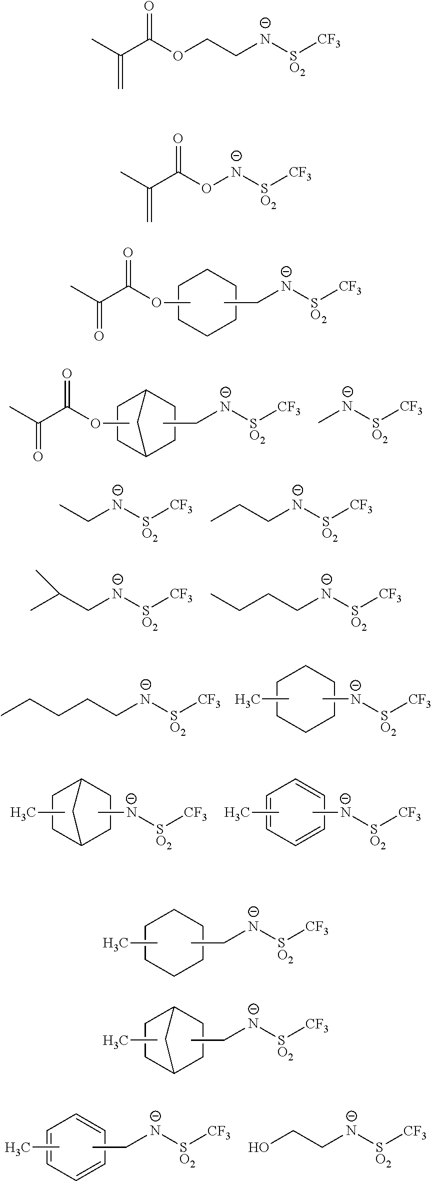

### Cation Moiety

In Formula (d1-3), M^{m+} represents an m-valent organic cation and has the same definition as that for M^{m+} in Formula (d1-1).

The component (d1-3) may be used alone or in combination of two or more kinds thereof.

Specific suitable examples of the component (D) in the resist composition according to the present embodiment are shown below.

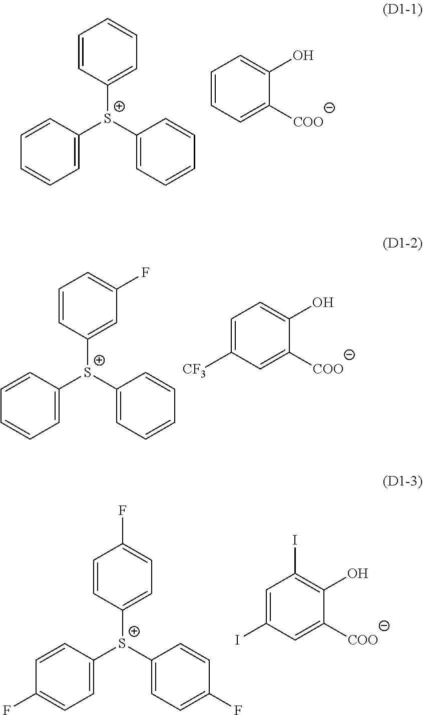

In the resist composition according to the present embodiment, as the (D) component, it is preferable to use at least one selected from the group consisting of the compounds represented by each of Chemical Formulae (D1-1) to (D1-3). Among these, from the viewpoint of improving all of sensitivity, roughness characteristics, and etching resistance, it is more preferable to use at least one selected from the group consisting of the compounds represented by each of Chemical Formulae (D1-2) to (D1-3), and it is still more preferable to use the compound represented by Chemical Formula (D1-3).

As the component (D1), only one of the above-described components (d1-1) to (d1-3) or a combination of two or more kinds thereof may be used.

In a case where the resist composition contains the component (D1), the content of the component (D1) in the resist composition is preferably in a range of 0.5 to 25 parts by mass, more preferably in a range of 1 to 20 parts by mass, and still more preferably in a range of 3 to 15 parts by mass with respect to 100 parts by mass of the component (A1).

In a case where the content of the component (D1) is greater than or equal to the lower limits of the above-described preferable ranges, particularly satisfactory lithography characteristics and a particularly satisfactory resist pattern shape are likely to be obtained. Meanwhile, in a case where the content thereof is less than or equal to the upper limits of the above-described preferable ranges, the sensitivity can be satisfactorily maintained, and the throughput is also excellent.

In the resist composition according to the present embodiment, it is preferable that the component (D1) contains the component (d1-1).

The content of the component (d1-1) in the entire component (D) contained in the resist composition according to the present embodiment is preferably 50% by mass or greater, more preferably 70% by mass or greater, and still more preferably 90% by mass or greater, and the component (D) may consist of only the component (d1-1) (100% by mass).

### Method for Producing Component (D1):

The methods of producing the component (d1-1) and the component (d1-2) are not particularly limited, and these components can be produced by known methods.

Further, the method for producing the component (d1-3) is not particularly limited, and the component is produced by the same method as disclosed in United States Paten Application, Publication No. 2012-0149916.

### In Regard to Component (D2)

The component (D) may contain a nitrogen-containing organic compound component (hereinafter, referred to as “component (D2)”) that does not correspond to the component (D1) described above.

The component (D2) is not particularly limited as long as the component functions as an acid diffusion control agent and does not correspond to the component (D1), and an optional component may be selected from known components and then used. Among the examples, an aliphatic amine is preferable, and particularly a secondary aliphatic amine and a tertiary aliphatic amine are more preferable.

The aliphatic amine is an amine containing one or more aliphatic groups, and the number of carbon atoms in the aliphatic group is preferably in a range of 1 to 12.

Examples of the aliphatic amine include amines in which at least one hydrogen atom of ammonia NH3 has been substituted with an alkyl group or hydroxyalkyl group having 12 or less carbon atoms (alkylamines or alkylalcoholamines), and cyclic amines.

Specific examples of the alkylamines and the alkylalcoholamines include monoalkylamines such as n-hexylamine, n-heptylamine, n-octylamine, n-nonylamine, and n-decylamine; dialkylamines such as diethylamine, di-n-propylamine, di-n-heptylamine, di-n-octylamine, and dicyclohexylamine; trialkylamines such as trimethylamine, triethylamine, tri-n-propylamine, tri-n-butylamine, tri-n-pentylamine, tri-n-hexylamine, tri-n-heptylamine, tri-n-octylamine, tri-n-nonylamine, tri-n-decylamine, and tri-n-dodecylamine; and alkylalcoholamines such as diethanolamine, triethanolamine, diisopropanolamine, triisopropanolamine, di-n-octanolamine, and tri-n-octanolamine. Among these, a trialkylamine having 6 to 30 carbon atoms is still more preferable, and tri-n-pentylamine or tri-n-octylamine is particularly preferable.

Examples of the cyclic amine include a heterocyclic compound having a nitrogen atom as a heteroatom. The heterocyclic compound may be a monocyclic compound (aliphatic monocyclic amine) or a polycyclic compound (aliphatic polycyclic amine).

Specific examples of the aliphatic monocyclic amine include piperidine and piperazine.

The aliphatic polycyclic amine preferably has 6 to 10 carbon atoms, and specific examples thereof include 1, 5-diazabicyclo[4.3.01-5-nonene, 1,8-diazabicyclo[5.4.0]-7-undecene, hexamethylenetetramine, and 1,4-diazabicyclo[2.2.2]octane.

Examples of other aliphatic amines include tris(2-methoxymethoxyethyl)amine, tris(2-(2-methoxyethoxy)ethyl)amine, tris{2-(2-methoxyethoxymethoxy)ethyl)amine, tris(2-(1-methoxyethoxy)ethyl amine, tris(2-(1-ethoxyethoxy)ethyl amine, tris(2-(1-ethoxypropoxy)ethyl)amine, tris[2-{2-(2-hydroxyethoxy)ethoxy}ethyl]amine and triethanol amine triacetate, and triethanol amine triacetate is preferable.

As the component (D2), an aromatic amine may be used.

Examples of the aromatic amine include 4-dimethylaminopyridine, 2,6-di-tert-butylpyridine, pyrrole, indole, pyrazole, imidazole, and derivatives thereof, tribenzylamine, 2,6-diisopropylaniline, and N-tert-butoxycarbonylpyrrolidine.

Among the examples, the component (D2) is preferably an alkylamine and more preferably a trialkylamine having 5 to 10 carbon atoms.

The component (D2) may be used alone or in combination of two or more kinds thereof.

In a case where the resist composition contains the component (D2), the content of the component (D2) in the resist composition is preferably in a range of 0.01 to 5 parts by mass, more preferably in a range of 0.1 to 5 parts by mass, and still more preferably in a range of 0.5 to 5 parts by mass with respect to 100 parts by mass of the component (A1).

In a case where the content of the component (D2) is greater than or equal to the lower limits of the above-described preferable ranges, particularly satisfactory lithography characteristics and a particularly satisfactory resist pattern shape are likely to be obtained. Meanwhile, in a case where the content thereof is less than or equal to the upper limits of the above-described preferable ranges, the sensitivity can be satisfactorily maintained, and the throughput is also excellent.

<<At Least One Compound (E) Selected from Group Consisting of Organic Carboxylic Acids, Phosphorus Oxo Acids, and Derivatives Thereof>>

For the purpose of preventing any deterioration in sensitivity and improving the resist pattern shape and the post-exposure temporal stability, the resist composition according to the present embodiment can contain, as an optional component, at least one compound (E) (hereinafter referred to as “component (E)”) selected from the group consisting of an organic carboxylic acid, and a phosphorus oxo acid and a derivative thereof.

Specific examples of the organic carboxylic acid include acetic acid, malonic acid, citric acid, malic acid, succinic acid, benzoic acid, and salicylic acid. Among these, salicylic acid is preferable.

Examples of the phosphorus oxo acid include phosphoric acid, phosphonic acid, and phosphinic acid. Among these, phosphonic acid is particularly preferable.

In the resist composition of the present embodiment, the component (E) may be used alone or in combination of two or more kinds thereof.

In a case where the resist composition contains the component (E), the content of the component (E) is preferably in a range of 0.01 to 5 parts by mass and more preferably in a range of 0.05 to 3 parts by mass with respect to 100 parts by mass of the component (A1). In a case where the content thereof is in the above-described ranges, the lithography characteristics are further improved.

<<Fluorine additive component (F)>>

The resist composition according to the present embodiment may contain a fluorine additive component (hereinafter, referred to as “component (F)”) as a hydrophobic resin. The component (F) is used to impart water repellency to the resist film, where it is used as a resin different from the component (A) to improve lithography characteristics.

As the component (F), for example, the fluorine-containing polymer compounds described in Japanese Unexamined Patent Application, First Publication Nos. 2010-002870, 2010-032994, 2010-277043, 2011-13569, and 2011-128226 can be used.

Specific examples of the component (F) include a polymer having a constitutional unit (f1) represented by General Formula (f1-1). As the polymer, a polymer (homopolymer) formed of only the constitutional unit (f1) represented by Formula (f1-1); a copolymer of the constitutional unit (f1) and the constitutional unit (a1); or a copolymer of the constitutional unit (f1), a constitutional unit derived from acrylic acid or methacrylic acid, and the constitutional unit (a1) is preferable, and a copolymer of the constitutional unit (f1) and the constitutional unit (a1) is more preferable. Here, as the constitutional unit (a1) copolymerized with the constitutional unit (f1), a constitutional unit derived from 1-ethyl-1-cyclooctyl (meth)acrylate or a constitutional unit derived from 1-methyl-1-adamantyl (meth)acrylate is preferable, and a constitutional unit derived from 1-ethyl-1-cyclooctyl (meth)acrylate is more preferable.

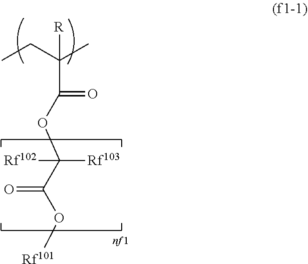

[In the formula, R has the same definition as described above, Rf¹⁰² and Rf¹⁰³ each independently represent a hydrogen atom, a halogen atom, an alkyl group having 1 to 5 carbon atoms, or a halogenated alkyl group having 1 to 5 carbon atoms, and Rf¹⁰² and Rf¹⁰³ may be the same as or different from each other, nf¹ represents an integer of 0 to 5, and Rf¹⁰¹ represents an organic group having a fluorine atom.]

In Formiula (f1-1), R bonded to the carbon atom at the α-position has the same definition as described above. It is preferable that R represents a hydrogen atom or a methyl group.

In Formula (f1-1), a fluorine atom is preferable halogen at the an atom as Rf¹⁰² and Rf¹⁰³. Examples of the alkyl group having 1 to 5 carbon atoms as Rf¹⁰² and Rf¹⁰³ include the same groups as those for the alkyl group having 1 to 5 carbon atoms as R. Among the examples, a methyl group or an ethyl group is preferable. Specific examples of the halogenated alkyl group having 1 to 5 carbon atoms as Rf¹⁰² and Rf¹⁰³ include groups in which some or all hydrogen atoms of an alkyl group having 1 to 5 carbon atoms are substituted with halogen atoms. Among these, a fluorine atom is preferable as the halogen atoms. Among these, Rf¹⁰² and Rf¹⁰³ sent preferably a hydrogen atom, a fluorine atom, or an alkyl group having 1 to 5 carbon atoms, more preferably a hydrogen atom, a fluorine atom, a methyl group, or an ethyl group, and still more preferably a hydrogen atom.

In Formula (f1-1), nf¹ represents an integer of 0 to 5, preferably an integer of 0 to 3, and more preferably 1 or 2.

In Formula (f1-1), Rf¹⁰¹ represents an organic group having a fluorine atom and preferably a hydrocarbon group having a fluorine atom.

The hydrocarbon group having a fluorine atom may be linear, branched, or cyclic, and the number of carbon atoms thereof is preferably in a range of 1 to 20, more preferably in a range of 1 to 15, and still more preferably in a range of 1 to 10.

In the hydrocarbon group having a fluorine atom, preferably 25% or more of the hydrogen atoms in the hydrocarbon group are fluorinated, more preferably 50% or more thereof are fluorinated, and particularly preferably 60% or more thereof are fluorinated from the viewpoint of increasing the hydrophobicity of the resist film during immersion exposure.

Among the examples, Rf¹⁰¹ represents more preferably a fluorinated hydrocarbon group having 1 to 6 carbon atoms and particularly preferably a trifluoromethyl group, —CH₂—CF₃, —CH₂—CF₂—CF₃, —CH(CF₃)₂, —CH₂—CH₂—CF₃, or —CH₂— CH₂—CF₂—CF₂—CF₂—CF₃.

The weight-average molecular weight (Mw) (in terms of polystyrene according to gel permeation chromatography) of the component (F) is preferably in a range of 1000 to 50000, more preferably in a range of 5000 to 40000, and most preferably in a range of 10000 to 30000. In a case where the weight-average molecular weight thereof is less than or equal to the upper limits of the above-described ranges, the resist composition exhibits a satisfactory solubility in a solvent for a resist enough to be used as a resist. Meanwhile, in a case where the weight-average molecular weight thereof is greater than or equal to the lower limits of the above-described ranges, water repellency of the resist film is improved.

Further, the dispersity (Mw/Mn) of the component (F) is preferably in a range of 1.0 to 5.0, more preferably in a range of 1.0 to 3.0, and most preferably in a range of 1.0 to 2.5.

In the resist composition according to the present embodiment, the component (F) may be used alone or in combination of two or more kinds thereof.

In a case where the resist composition contains the component (F), the content of the component (F) is preferably in a range of 0.5 to 10 parts by mass and more preferably in a range of 1 to 10 parts by mass with respect to 100 parts by mass of the component (A1).

### <<Organic Solvent Component (S)>>

The resist composition of the present embodiment can be produced by dissolving the resist materials in an organic solvent component (hereinafter, referred to as “component (S)”).

The component (S) may be any organic solvent which can dissolve each component to be used to obtain a uniform solution, and an optional organic solvent can be appropriately selected from those which have been known as solvents of a chemically amplified resist composition and then used.

Examples of the component (S) include lactones such as y-butyrolactone: ketones such as acetone, methyl ethyl ketone, cyclohexanone, methyl-n-pentyl ketone, methyl isopentyl ketone, and 2-heptanone; polyhydric alcohols such as ethylene glycol, diethylene glycol, propylene glycol, and dipropylene glycol; compounds having an ester bond, such as ethylene glycol monoacetate, diethylene glycol monoacetate, propylene glycol monoacetate, and dipropylene glycol monoacetate; polyhydric alcohol derivatives of compounds having an ether bond such as monoalkyl ether or monophenyl ether, such as monomethylether, monoethylether, monopropylether, or monobutylether of polyhydric alcohols or compounds having an ester bond [among these, propylene glycol monomethyl ether acetate (PGMEA) and propylene glycol monomethyl ether (PGME) are preferable]; cyclic ethers such as dioxane; esters such as methyl lactate, ethyl lactate (EL), methyl acetate, ethyl acetate, butyl acetate, methyl pyruvate, ethyl pyruvate, methyl methoxypropionate, and ethyl ethoxypropionate; aromatic organic solvents such as anisole, ethylbenzylether, cresylniethylether, diphenylether, dibenzylether, phenetole, butylphenylether, ethylbenzene, diethylbenzene, pentylbenzene, isopropylbenzene, toluene, xylene, cymene, and mesitylene; and dimethylsulfoxide (DMSO).

In the resist composition of the present embodiment, the component (S) may be used alone or in the form of a mixed solvent of two or more kinds thereof. Among these, PGMEA, PGME, γ-butyrolactone, EL, or cyclohexanone is preferable.

Further, a mixed solvent obtained by mixing PGMEA with a polar solvent is also preferable as the component (S). The blending ratio (mass ratio) of the mixed solvent may be appropriately determined in consideration of the compatibility between PGMEA and the polar solvent, but is preferably in the range of 1:9 to 9:1 and more preferably in a range of 2:8 to 8:2.

More specifically, in a case where EL or cyclohexanone is blended as the polar solvent, the mass ratio of PGMEA to EL or cyclohexanone is preferably in a range of 1:9 to 9:1 and more preferably in a range of 2:8 to 8:2. Further, in a case where PGME is blended as the polar solvent, the mass ratio of PGMEA to PGME is preferably in a range of 1:9 to 9:1, more preferably in a range of 2:8 to 8:2, and still more preferably in a range of 3:7 to 7:3. Further, a mixed solvent of PGMEA, PGME, and cyclohexanone is also preferable.

Further, a mixed solvent of 7-butyrolactone and at least one selected from PGMEA and EL is also preferable as the component (S). In this case, as the mixing ratio, the mass ratio between the former and the latter is preferably in a range of 70:30 to 95:5.

The amount of the component (S) to be used is not particularly limited and is appropriately set to have a concentration which enables coating a substrate or the like depending on the thickness of the coated film. The component (S) is typically used in an amount such that the solid content concentration of the resist composition is set to be in a range of 0.1% to 20% by mass and preferably in a range of 0.2% to 15% by mass.

After the resist material is dissolved in the component (S), impurities may be removed from the resist composition of the present embodiment using a porous polyimide film, a porous polyamideimide film, or the like. For example, the resist composition may be filtered using a filter formed of a porous polyimide film, a filter formed of a porous polyamideimide film, a filter formed of a porous polyimide film and a porous polyamideimide film, or the like. Examples of the porous polyimide film and the porous polyamideimide film include those described in Japanese Unexamined Patent Application, First Publication No. 2016-155121.

As described above, the resist composition according to the present embodiment contains the resin component (A1) having the constitutional unit (a0) derived from a compound represented by General Formula (a0-m0).

The constitutional unit (a0) has a —Ra⁰² group and an acid dissociable ester group (—C(═O)—O—Ra⁰¹) in the aromatic hydrocarbon group (W⁰²). Ra⁰² represents an iodine atom or a bromine atom. Ra⁰¹ represents a group having an alicyclic group, which has no carbon-carbon unsaturated bond, a group having a carbon-carbon unsaturated bond, or a group having an aromatic group.

In a case where the aromatic hydrocarbon group (W⁰²) has an iodine atom or a bromine atom, in the step of performing exposure, the light absorption efficiency and the reactivity are enhanced.

In addition, in a case where the aromatic hydrocarbon group (W⁰²) further has Ra⁰¹, the carbon density of the resist composition according to the present embodiment is further increased.

With these synergistic actions, in the resist composition according to the present embodiment, which contains the resin component (A1) having the constitutional unit (a0), the sensitivity, the roughness characteristic, and the etching resistance can be all improved.

### (Resist Pattern Formation Method)

A resist pattern formation method according to the second aspect of the present invention is a method including a step of forming a resist film on a support using the resist composition according to the first aspect of the present invention described above, a step of exposing the resist film to light, and a step of developing the resist film exposed to light to form a resist pattern.

According to the embodiment of the resist pattern formation method, a resist pattern formation method by performing processes as described below is an exemplary example.

First, a support is coated with the resist composition of the above-described embodiment using a spinner or the like, and a bake (post applied bake (PAB)) treatment is performed under a temperature condition of 80° C. to 150° C. for 40 to 120 seconds and preferably 60 to 90 seconds to form a resist film.

Next, the selective exposure is performed on the resist film by, for example, light exposure through a mask (mask pattern) having a predetermined pattern formed thereon using an exposure apparatus such as an electron beam lithography apparatus or an ArF exposure apparatus, or direct irradiation with an electron beam for drawing without using a mask pattern, and a bake treatment (post exposure bake (PEB)) is carried out, for example, under a temperature condition of 80° C. to 150° C. for 40 to 120 seconds and preferably 60 to 90 seconds.

Next, the resist film is subjected to a developing treatment. The developing treatment is conducted using an alkali developing solution in a case of an alkali developing process and using a developing solution containing an organic solvent (organic developing solution) in a case of a solvent developing process.

After the developing treatment, it is preferable to conduct a rinse treatment. As the rinse treatment, water rinsing using pure water is preferable in a case of the alkali developing process, and rinsing using a rinse solution containing an organic solvent is preferable in a case of the solvent developing process.

In a case of the solvent developing process, the developing solution or the rinse solution attached onto the pattern may be removed by a treatment using a supercritical fluid after the developing treatment or the rinse treatment.

After the developing treatment or the rinse treatment, drying is conducted. As desired, a bake treatment (post bake) may be conducted after the developing treatment.

In this manner, a resist pattern can be formed.

The support is not particularly limited and a known support of the related art can be used, and examples thereof include a substrate for an electronic component and a substrate on which a predetermined wiring pattern has been formed. Specific examples thereof include a metal substrate such as a silicon wafer, copper, chromium, iron, or aluminum; and a glass substrate. As the materials of the wiring pattern, copper, aluminum, nickel, or gold can be used.

The wavelength used for light exposure is not particularly limited and the exposure can be conducted using radiation such as an ArF excimer laser, a KrF excimer laser, an F2 excimer laser, extreme ultraviolet rays (EUV), vacuum ultraviolet rays (VUV), electron beams (EB), X-rays, and soft X-rays.

The resist composition according to the above-described embodiment is highly useful for a KrF excimer laser, an ArF excimer laser, EB, and EUV, more useful for an ArF excimer laser, EB, and EUV, and particularly useful for EB and EUV. That is, the resist pattern formation method according to the present embodiment is a method particularly useful in a case where the step of exposing the resist film to light includes a process of exposing the resist film to extreme ultraviolet rays (EUV) or electron beams (EB).

The method for exposing the resist film to light may be a general exposure (dry exposure) conducted in air or an inert gas such as nitrogen, or liquid immersion exposure (liquid immersion lithography).

The liquid immersion exposure is an exposure method in which the region between the resist film and the lens at the lowermost position of the exposure apparatus is filled with a solvent (liquid immersion medium) in advance that has a refractive index greater than the refractive index of air, and the exposure (immersion exposure) is conducted in this state.

The liquid inunersion medium is preferably a solvent that exhibits a refractive index greater than the refractive index of air but less than the refractive index of the resist film to be exposed, and examples thereof include water, a fluorine-based inert liquid, a silicon-based solvent, and a hydrocarbon-based solvent. As the liquid immersion medium, water is suitably used.

As the alkali developing solution used for the developing treatment in the alkali developing process, a 0.1 to 10 mass % tetramethylammonium hydroxide (TMAH) aqueous solution is an exemplary example.

The content of the organic solvent in the organic developing solution used for the developing treatment in the solvent developing process is typically 90% by mass or greater, and may be 95% by mass or greater, 98% by mass or greater, 100% by mass, and preferably 100% by mass with respect to the total amount of the organic developing solution.

The organic solvent contained in the organic developing solution may be obtained by dissolving the component (A) (component (A) before exposure) and can be appropriately selected from known organic solvents. Specific examples thereof include a polar solvent such as a ketone-based solvent, an ester-based solvent, an alcohol-based solvent, a nitrile-based solvent, an amide-based solvent, and an ether-based solvent, and a hydrocarbon-based solvent.

Examples of the ester-based solvent include methyl acetate, butyl acetate, ethyl acetate, isopropyl acetate, pentyl acetate, isopentyl acetate, amyl acetate, propylene glycol monomethyl ether acetate, ethylene glycol monoethyl ether acetate, diethylene glycol monobutyl ether acetate, diethylene glycol monoethyl ether acetate, ethyl-3-ethoxypropionate, 3-methoxybutyl acetate, 3-methyl-3-methoxybutyl acetate, methyl formate, ethyl formate, butyl formate, propyl formate, ethyl lactate, butyl lactate, propyl lactate, butyl butanoate, methyl 2-hydroxyisobutyrate, isoamyl acetate, isobutyl isobutyrate, and butyl propionate.

Examples of the nitrile-based solvent include acetonitrile, propionitrile, valeronitrile, and butyronitrile.

Known additives can be blended into the organic developing solution as necessary. Examples of the additive include a surfactant. The surfactant is not particularly limited, and for example, an ionic or non-ionic fluorine-based and/or silicon-based surfactant can be used. As the surfactant, a non-ionic surfactant is preferable, and a non-ionic fluorine-based surfactant or a non-ionic silicon-based surfactant is more preferable.

In a case where a surfactant is blended into the solution, the amount of the surfactant to be blended is typically in a range of 0.001% to 5% by mass, preferably in a range of 0.005% to 2% by mass, and more preferably in a range of 0.01% to 0.5% by mass with respect to the total amount of the organic developing solution.

The developing treatment can be performed according to a known developing method, and examples thereof include a method for immersing a support in a developing solution for a certain time (a dip method), a method for raising a developing solution on the surface of a support using the surface tension and maintaining the state for a certain time (a puddle method), a method for spraying a developing solution to the surface of a support (spray method), and a method for continuously ejecting a developing solution onto a support rotating at a certain rate while scanning a developing solution ejection nozzle at a certain rate (dynamic dispense method).

As the organic solvent contained in the rinse solution used for the rinse treatment after the developing treatment in the solvent developing process, a solvent that is unlikely to dissolve a resist pattern can be appropriately selected from the organic solvents described as the organic solvent used in the organic developing solution and then used. Typically, at least one solvent selected from a hydrocarbon-based solvent, a ketone-based solvent, an ester-based solvent, an alcohol-based solvent, an amide-based solvent, and an ether-based solvent is used. Among these, at least one solvent selected from a hydrocarbon-based solvent, a ketone-based solvent, an ester-based solvent, an alcohol-based solvent, and an amide-based solvent is preferable, at least one solvent selected from an alcohol-based solvent and an ester-based solvent is more preferable, and an alcohol-based solvent is particularly preferable.

As the alcohol-based solvent used in the rinse solution, a monohydric alcohol having 6 to 8 carbon atoms is preferable, and the monohydric alcohol may be linear, branched, or cyclic. Specific examples thereof include 1-hexanol, 1-heptanol, 1-octanol, 2-hexanol, 2-heptanol, 2-octanol, 3-hexanol, 3-heptanol, 3-octanol, 4-octanol, and benzyl alcohol. Among these, 1-hexanol, 2-heptanol, and 2-hexanol are preferable, and 1-hexanol and 2-hexanol are more preferable.

These organic solvents may be used alone or in combination of two or more kinds thereof. Further, an organic solvent other than the above-described solvents and water may be mixed and used. However, in consideration of the development characteristics, the amount of water to be blended into the rinse solution is preferably 30% by mass or less, more preferably 10% by mass or less, still more preferably 5% by mass or less, and particularly preferably 3% by mass or less with respect to the total amount of the rinse solution.

A known additive can be blended into the rinse solution as necessary. Examples of the additive include a surfactant. As the surfactant, the same surfactants as those described above are exemplary examples. Among these, a non-ionic surfactant is preferable, and a non-ionic fluorine-based surfactant or a non-ionic silicon-based surfactant is more preferable.

In a case where a surfactant is blended into the solution, the amount of the surfactant to be blended is typically in a range of 0.001% to 5% by mass, preferably in a range of 0.005% to 2% by mass, and more preferably in a range of 0.01% to 0.5% by mass with respect to the total amount of the rinse solution.

The rinse treatment carried out using a rinse solution (washing treatment) can be performed according to a known rinse method. Examples of the method for performing the rinse treatment include a method for continuously ejecting a rinse solution onto a support rotating at a certain rate (rotary coating method), a method for immersing a support in a rinse solution for a certain time (dip method), and a method for spraying a rinse solution to the surface of a support (spray method).

With the resist pattern formation method according to the present embodiment described above, since the above-described resist composition is used, it is possible to easily form a resist pattern in which all of the sensitivity, the roughness characteristics, and the etching resistance are satisfactory.

Various materials that are used in the resist composition according to the above-described embodiment and the resist pattern formation method according to the above-described embodiment (for example, a resist solvent, a developing solution, a rinse liquid, a composition for forming an antireflection film, and a composition for forming a top coat) preferably do not contain impurities such as a metal, a metal salt containing halogen, an acid, an alkali, and a component containing a sulfur atom or phosphorus atom.

Here, examples of the impurities containing metal atoms include Na, K, Ca, Fe, Cu, Mn, Mg, A1. Cr, Ni, Zn, Ag, Sn, Pb, Li, and salts thereof. The content of the impurities contained in these materials is preferably 200 ppb or less, more preferably 1 ppb or less, still more preferably 100 parts per trillion (ppt) or less, particularly preferably 10 ppt or less, and most preferably substantially zero (less than or equal to the detection limit of the measuring device).

(Compound represented by General Formula (a0-m0))

The compound according to a third aspect of the present invention is a compound represented by General Formula (a0-m0).

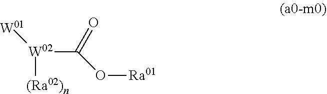

[in the formula, W⁰¹ represents a polymerizable group-containing group. W⁰² represents an aromatic hydrocarbon group which may have a substituent. Ra⁰¹ is an acid dissociable group and represents a group having an alicyclic group, which has no carbon-carbon unsaturated bond, a group having a carbon-carbon unsaturated bond, or a group having an aromatic group. The alicyclic group and the aromatic group may each have a substituent. Ra⁰² represents an iodine atom or a bromine atom, n represents an integer of 1 or greater.]

The compound represented by General Formula (a0-m0) is the same as the compound represented by General Formula (a0-m0) in the resist composition according to the embodiment described above.

It is suitable that W⁰¹ represents, for example, a group represented by Chemical Formula: C(R^{X11})(R^{X12})═C(R^{X13})—Ya^{x0}—. In the chemical formula, R^{X11}, R^{X12}, and R^{X13} each represent a hydrogen atom, an alkyl group having 1 to 5 carbon atoms, or a halogenated alkyl group having 1 to 5 carbon atoms, and Ya^{x0} represents a single bond or a divalent linking group.

R^{X11} and R^{X12} each represent preferably a hydrogen atom, an alkyl group having 1 to 5 carbon atoms, or a fluorinated alkyl group having 1 to 5 carbon atoms, and from the viewpoint of industrial availability, more preferably a hydrogen atom or a methyl group, and particularly preferably a hydrogen atom.

In addition, R^{X13} represents preferably a hydrogen atom, an alkyl group having 1 to 5 carbon atoms, or a fluorinated alkyl group having 1 to 5 carbon atoms, and from the viewpoint of industrial availability, more preferably a hydrogen atom or a methyl group, and particularly preferably a hydrogen atom. Ya¹⁰ represents more preferably an ester bond [—C(═O)—O— or —O—C(═O)—] or a single bond and still more preferably a single bond.

Suitable examples of Rd⁰¹ include the “acetal type acid dissociable group”, the “tertiary alkyl ester type acid dissociable group”, and the “secondary alkyl ester type acid dissociable group” described above, and among these, the “tertiary alkyl ester type acid dissociable group” and the “secondary alkyl ester type acid dissociable group” are more preferable, and the acid dissociable group represented by General Formula (a1-r-2) or the acid dissociable group represented by General Formula (a1-r-4) is preferable.

Ra² represents an iodine atom or a bromine atom, n is an integer of 1 or greater, preferably an integer of 1 to 3, more preferably 1 or 2, and particularly preferably 2.

The compound according to the present embodiment is preferably a compound represented by General Formula (a0-m1).

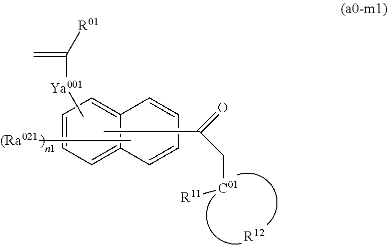

[In the formula. R⁰¹ represents an alkyl group having 1 to 5 carbon atoms, a halogenated alkyl group having 1 to 5 carbon atoms, or a hydrogen atom. Ya⁰⁰¹ represents a divalent linking group or a single bond. C¹ represents a tertiary carbon atom. R¹¹ represents a chain-like saturated hydrocarbon group which may have a substituent. R¹² represents a group forming an alicyclic group with C¹, which has no carbon-carbon unsaturated bond. The alicyclic group here may have a substituent. Ra⁰²¹ represents an iodine atom or a bromine atom. q1 represents an integer of 0 to 3, n1 represents an integer of 1 or greater. Provided that, n1≤q1×2+4 is satisfied. The benzene ring in Formula (a0-m1) may have a substituent other than Ra⁰²¹.]

In Formula (a0-m1), as the alkyl group having 1 to 5 carbon atoms as R⁰¹, a linear or branched alkyl group having 1 to 5 carbon atoms is preferable, and specific examples thereof include a methyl group, an ethyl group, a propyl group, an isopropyl group, an n-butyl group, an isobutyl group, a tert-butyl group, a pentyl group, an isopentyl group, and a neopentyl group. The halogenated alkyl group having 1 to 5 carbon atoms is a group in which some or all hydrogen atoms in the alkyl group having 1 to 5 carbon atoms have been substituted with halogen atoms. As the halogen atom, a fluorine atom is particularly preferable.

R⁰¹ represents preferably a hydrogen atom, an alkyl group having 1 to 5 carbon atoms, or a fluorinated alkyl group having 1 to 5 carbon atoms, and it is most preferably a hydrogen atom or a methyl group in terms of industrial availability.

In Formula (a0-m1), Ya⁰⁰¹, Ra⁰²¹, R¹¹, R¹², n1, and q1 are each the same as Ya⁰, Ra⁰²¹, R¹¹, R¹², n1, and q1 in General Formula (a0-1) described above.

Preferred specific examples of the compound represented by General Formula (a0-m1) are shown below.

In the formulae shown below, R^{α} represents a hydrogen atom, a methyl group, or a trifluoromethyl group. R⁰²⁰ represents an iodine atom or a bromine atom.

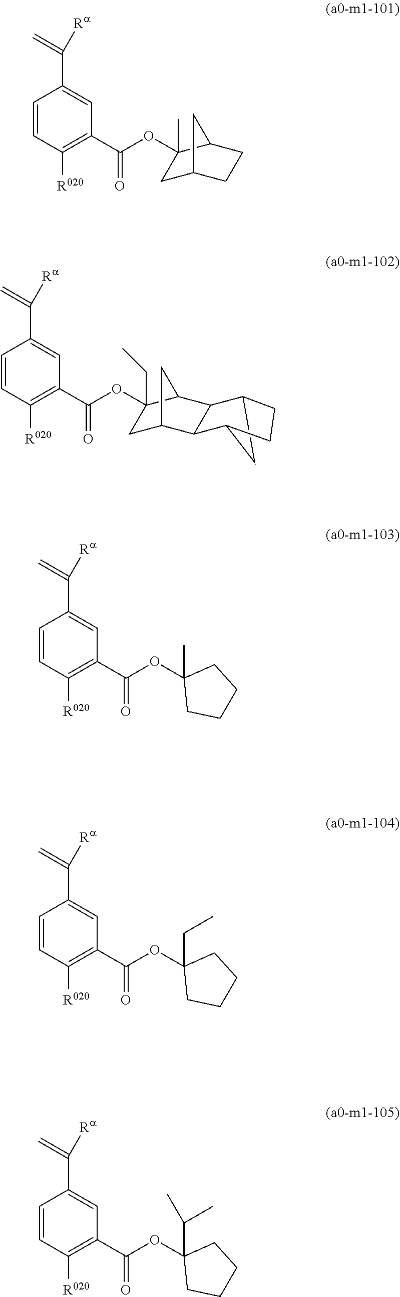

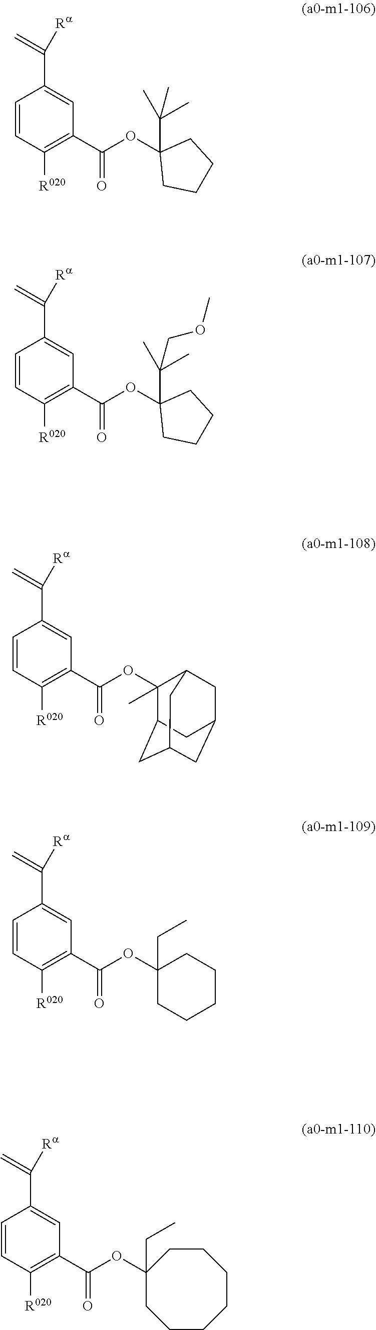

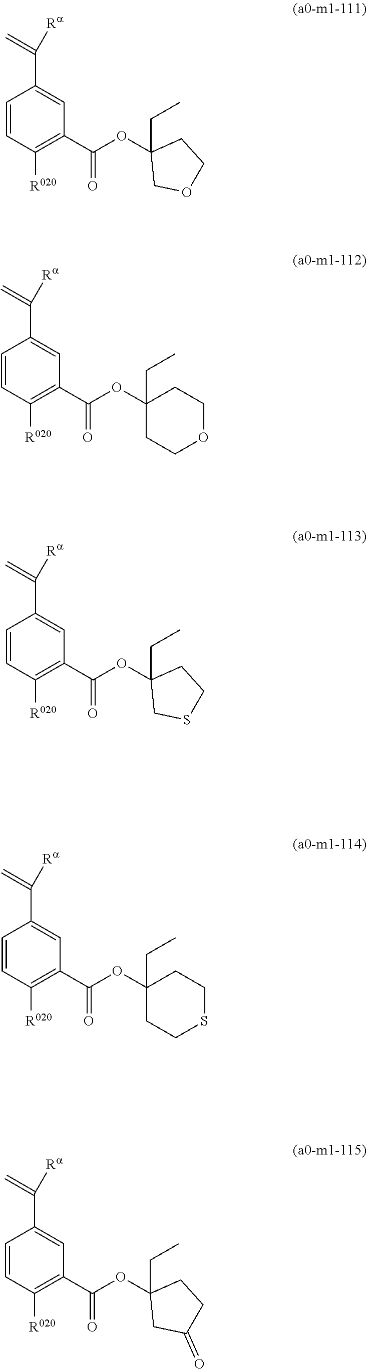

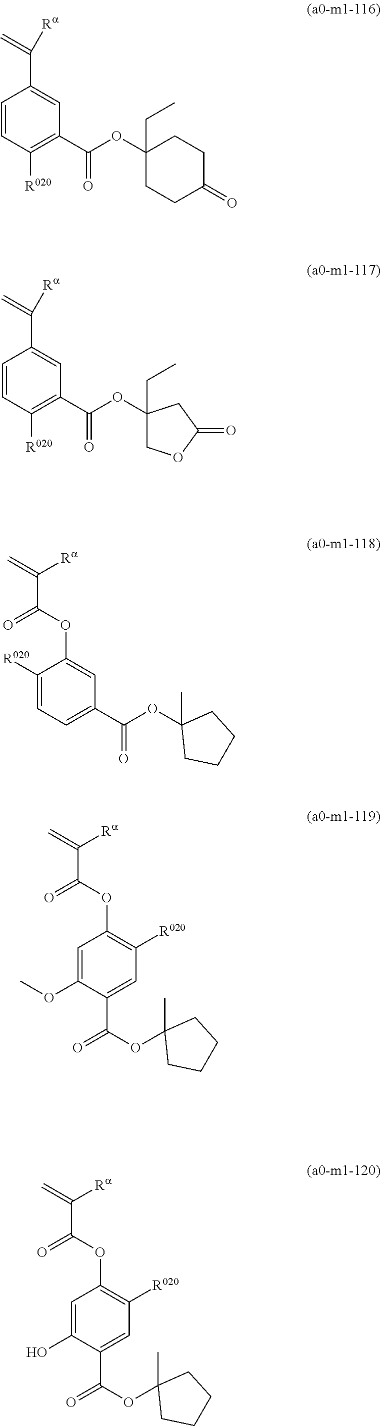

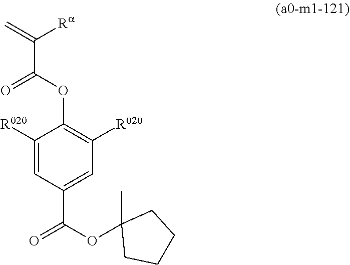

The compound according to the present embodiment is preferably a compound represented by General Formula (a0-m2).

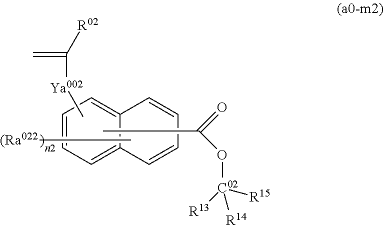

[In the formula, R(² represents an alkyl group having 1 to 5 carbon atoms, a halogenated alkyl group having 1 to 5 carbon atoms, or a hydrogen atom. Ya⁰⁰² represents a divalent linking group or a single bond. C⁰² represents a secondary carbon atom or a tertiary carbon atom, and any of α-positions of C⁰² represents a carbon atom constituting a carbon-carbon unsaturated bond with a β-position carbon atom or a carbon atom constituting an aromatic ring. R¹³ represents an aromatic group which may have a substituent, a chain-like unsaturated hydrocarbon group which may have a substituent, or a hydrogen atom. R¹⁴ and R¹⁵ each independently represent a chain-like hydrocarbon group which may have a substituent or a hydrogen atom, or R¹⁴ and R¹⁵ are bonded to each other to form a cyclic group which may have a substituent. Ra⁰²² represents an iodine atom or a bromine atom. q2 represents an integer of 0 to 3, n2 represents an integer of 1 or greater. Provided that, n2 S q2×2+4 is satisfied. The benzene ring in Formula (a0-m2) may have a substituent other than Ra⁰²².]

In Formula (a0-m2), —C⁰²(R¹)(R¹⁴)(R¹⁵) group represents an acid dissociable group. C⁰² represents a secondary carbon atom or a tertiary carbon atom.

In Formula (a0-m2), in a case where C⁰² represents a secondary carbon atom, any one of R¹³, R¹⁴, or R¹⁵ represents a hydrogen atom.

In a case where R¹³ represents a hydrogen atom, R¹⁴ and R¹⁵ each independently represent a chain-like hydrocarbon group which may have a substituent, or R¹⁴ and R^{1S} may be bonded to each other to form a cyclic group which may have a substituent.

Alternatively, in a case where one of R¹⁴ or R¹⁵ represents a hydrogen atom, the other represents a chain-like hydrocarbon group which may have a substituent, and R¹³ represents an aromatic group which may have a substituent or a chain-like unsaturated hydrocarbon group which may have a substituent.

In Formula (a0-m2), in a case where C⁰² represents a tertiary carbon atom, R¹³, R⁴, and R″ are not all hydrogen atoms.

R¹³ represents an aromatic group which may have a substituent or a chain-like unsaturated hydrocarbon group which may have a substituent, and R¹⁴ and R^{1S} each independently represent a chain-like hydrocarbon group which may have a substituent or R¹⁴ and R¹⁵ may be bonded to each other to form a cyclic group which may have a substituent.

In Formula (a0-m2), as the alkyl group having 1 to 5 carbon atoms as R⁰², a linear or branched alkyl group having 1 to 5 carbon atoms is preferable, and specific examples thereof include a methyl group, an ethyl group, a propyl group, an isopropyl group, an n-butyl group, an isobutyl group, a tert-butyl group, a pentyl group, an isopentyl group, and a neopentyl group. The halogenated alkyl group having 1 to 5 carbon atoms is a group in which some or all hydrogen atoms in the alkyl group having 1 to 5 carbon atoms have been substituted with halogen atoms. As the halogen atom, a fluorine atom is particularly preferable.

R⁰² represents preferably a hydrogen atom, an alkyl group having 1 to 5 carbon atoms, or a fluorinated alkyl group having 1 to 5 carbon atoms, and it is most preferably a hydrogen atom or a methyl group in terms of industrial availability.

In Formula (a0-m2), Ya⁰⁰², Ra²², R¹³, R¹⁴, R¹⁵, n2, and q2 are each the same as Ya⁰⁰², Ra⁰²², R¹³, R¹⁴, R¹⁵, n2, and q2 in General Formula (a0-2) described above.

Preferred specific examples of the compound represented by General Formula (a0-m2) are shown below.

In the formulae shown below, R″ represents a hydrogen atom, a methyl group, or a trifluoromethyl group. R⁰²⁰ represents an iodine atom or a bromine atom.

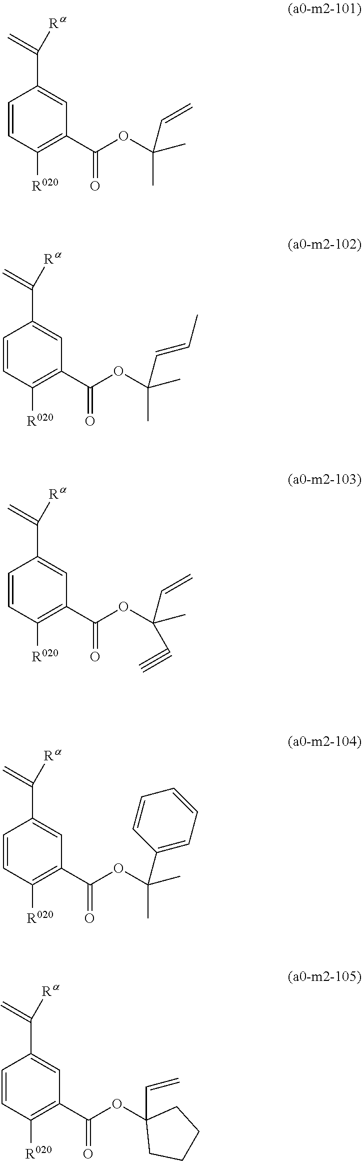

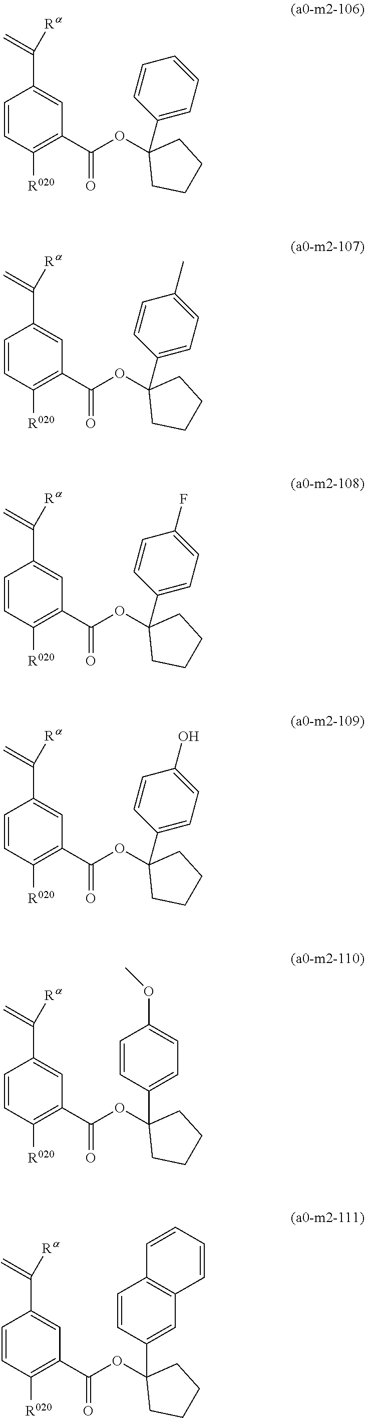

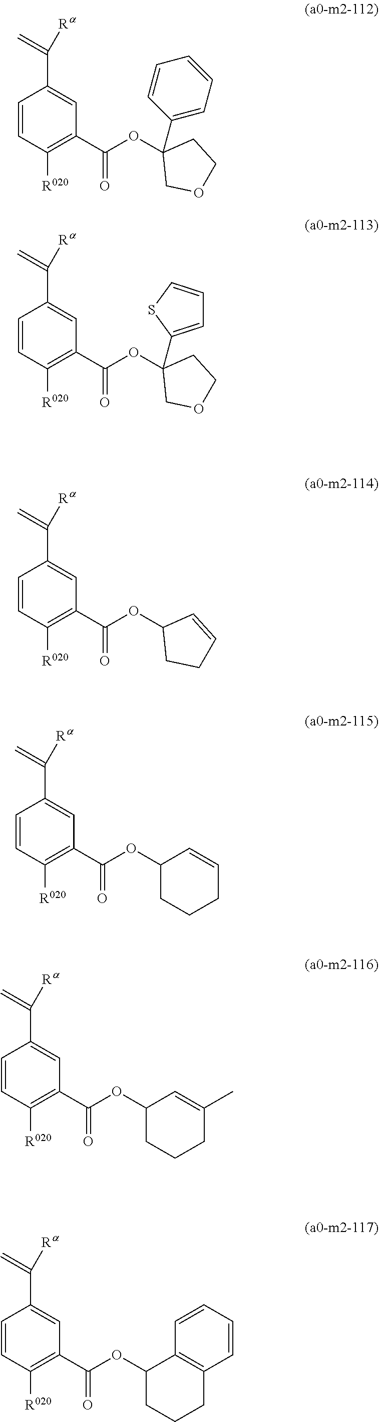

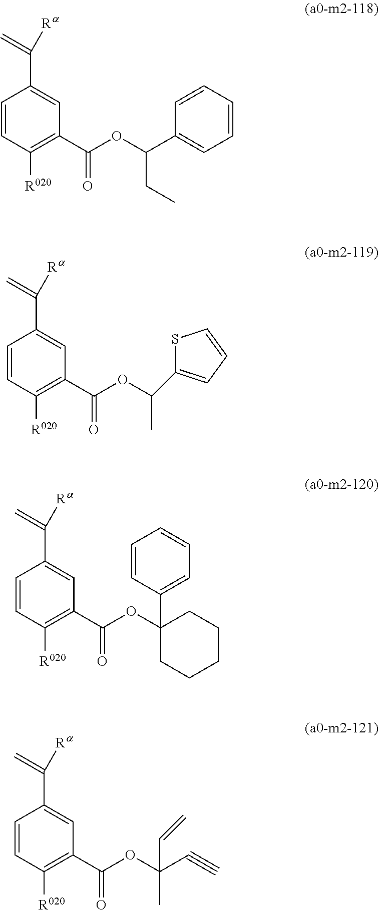

Specific examples of the substituent positions of the —C(═O)—O—Ra⁰¹ group and the Ra⁰² group in the aromatic hydrocarbon group (W⁰²) in General Formula (a0-m0) are shown below.

In the formulae shown below. R″ represents a hydrogen atom, a methyl group, or a trifluoromethyl group. R⁰¹ represents an optional acid dissociable group. Rm represents an iodine atom or a bromine atom.

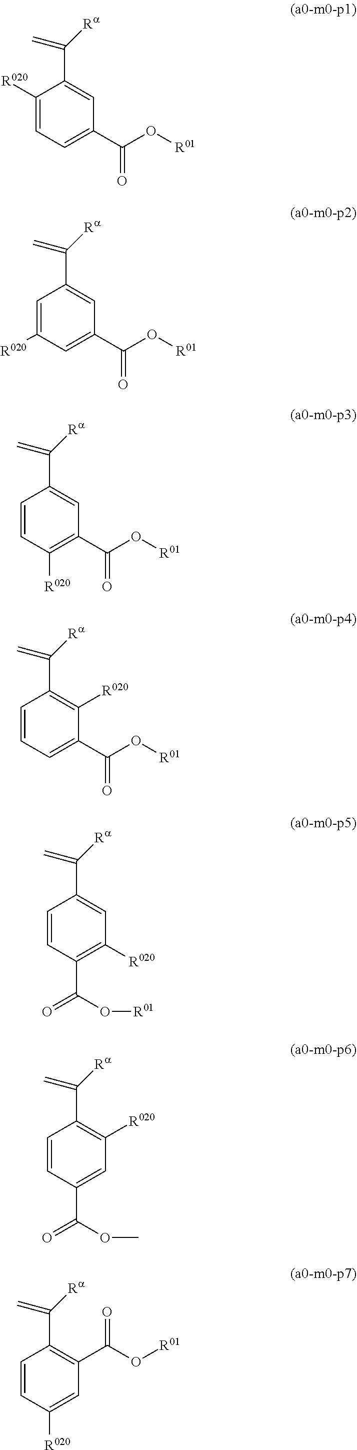

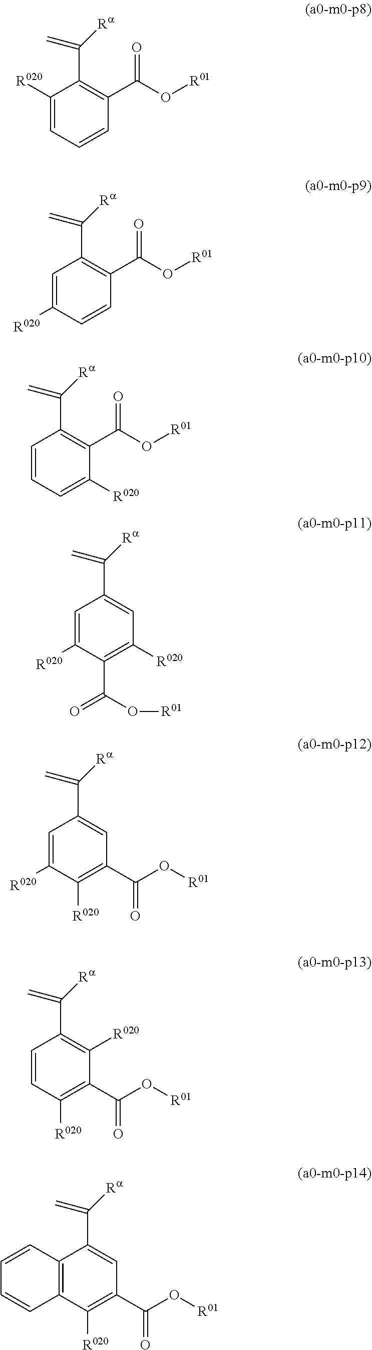

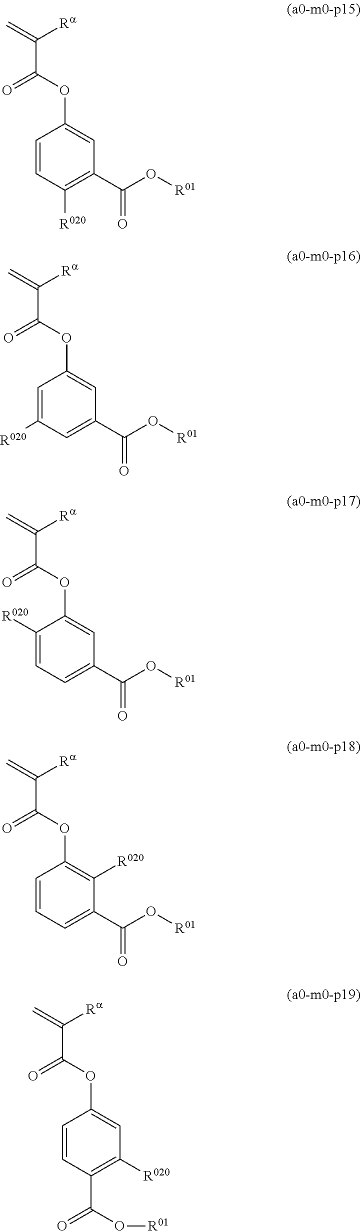

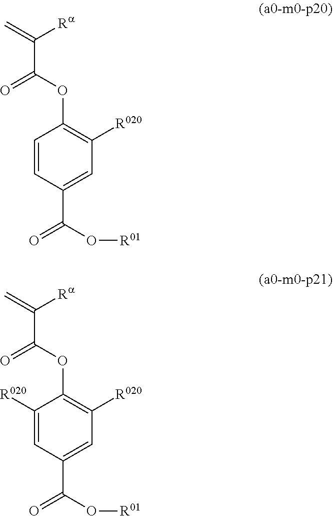

The aromatic hydrocarbon group (W2) in General Formula (a0-m0) may have a substituent R⁰³, in addition to the —C(═O)—O—Ra⁰¹ group and the Ra⁰² group.

In the formulae shown below, R″ represents a hydrogen atom, a methyl group, or a trifluoromethyl group. R⁰¹ represents an optional acid dissociable group. R⁰²⁰ represents an iodine atom or a bromine atom.

R⁰³ may be an alkyl group, a fluorine atom, a chlorine atom, a halogenated alkyl group, a hydroxyl group, an alkoxy group, a carbonyl group, a cyano group, an amino group, a nitro group, an aryl group, or the like.

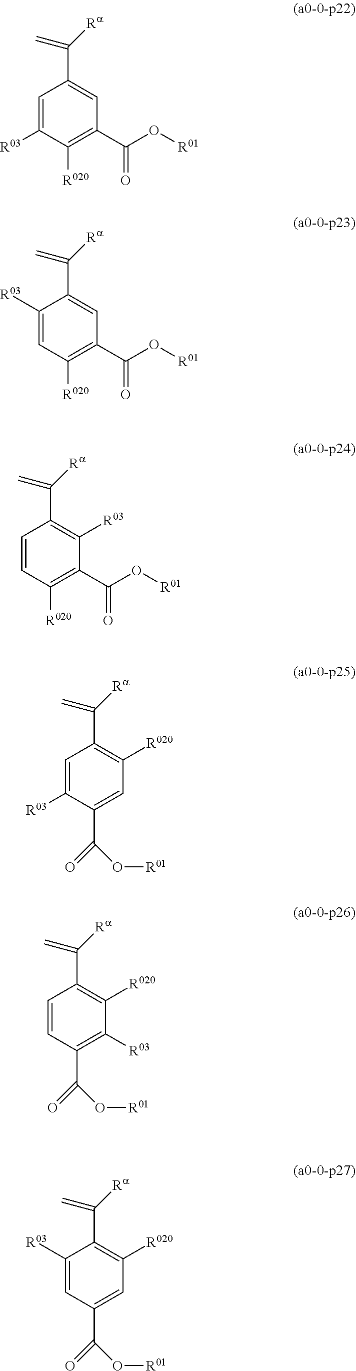

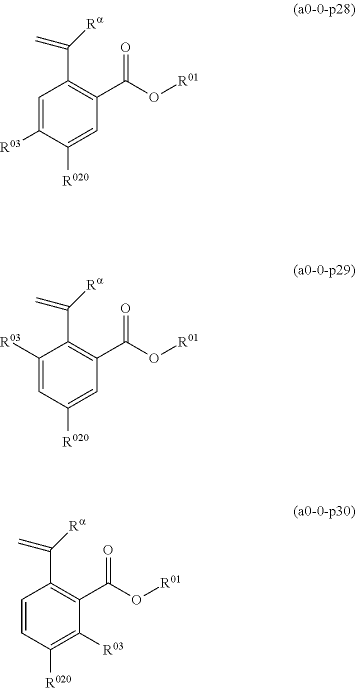

[Method for Producing Compound (a0-m0)]

The compound according to the present embodiment can be produced by, for example, the production method including the following esterification step.

In the esterification step, a compound represented by Formula (a0-m0-c1) is reacted with a compound represented by Formula (A1c-1) to obtain a compound (a0-m0).

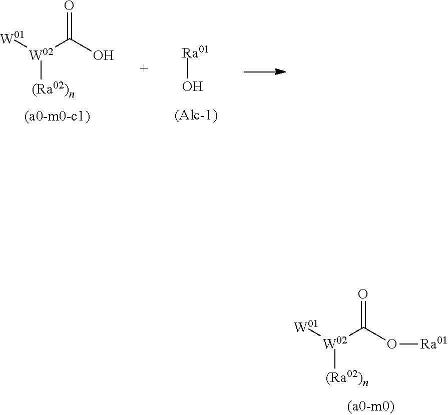

[In the formula, W⁰¹ represents a polymerizable group-containing group. W⁰² represents an aromatic hydrocarbon group which may have a substituent. Ra⁰² represents an iodine atom or a bromine atom. Ra⁰¹ represents an acid dissociable group, n represents an integer of 1 or greater.]

W⁰¹ is not particularly limited as long as W⁰¹ represents a polymerizable group-containing group, and examples thereof include *—C═C(R^{αx}), *—O—C(═O)—C═C(R^{α}), and the like. R^{α} represents a hydrogen atom, a methyl group, or a trifluoromethyl group. * represents a bonding site with W⁰².

A reaction temperature condition of the esterification step is not particularly limited, and is, for example, about 0° C. to 120° C.

The reaction time of the esterification step is not particularly limited, and is, for example, about 1 to 72 hours.

Examples of the reaction solvent that is used in the esterification step include dichloromethane, dichloroethane, chloroform, tetrahydrofuran, N,N-dimethylformamide, acetonitrile, propionitrile, N,N′-dimethylacetamide, and dimethylsulfoxide.

In addition, in the reaction of the esterification step, a condensing agent and a basic catalyst may be used.

Specific examples of the condensing agent include N,N′-dicyclohexylcarbodiimide, N,N′-diisopropylcarbodiimide, 1-ethyl-3-(3-dimethylaminopropyl)carbodiimide hydrochloride, and carbonyldiimidazole (CDI).

Specific examples of the basic catalyst include tertiary amines such as trimethylamine, triethylamine, and tributylamine, aromatic amines such as pyridine, dimethylaminopyridine, and pyrrolidinopyridine, diazabicyclononene (DBN), and diazabicycloundecene (DBU).

In the method of producing the compound (a0-m0) described above, the compound in the reaction solution may be isolated and purified after completion of each reaction. A known method in the related art can be used for isolation and purification, and for example, concentration, solvent extraction, distillation, crystallization, recrystallization, or chromatography can be appropriately combined and used.

The structure of the compound obtained as described above can be identified by typical organic analysis methods such as ¹H-nuclear magnetic resonance (NMR) spectroscopy, ¹³C-NMR spectroscopy, ¹⁹F-NMR spectroscopy, infrared (IR) absorption spectroscopy, mass spectrometry (MS), an elemental analysis method, and an X-ray crystal diffraction method.

As the raw material that is used in each step, a commercially available raw material may be used, or a synthetic raw material may be used.

In Formula (a0-m0-c1), the compound (a0-m0-c1 k) in a case where W⁰¹ represents *—C═C(R^{α}) can be synthesized, for example, by the following steps (i) and (ii).

### Step (i):

a step of reacting a compound represented by Formula (a0-m0-c3k) with a radical initiator and a halogenating agent to obtain a compound represented by Formula (a0-m0-c2k)

### Step (ii):

a step of reacting a compound represented by Formula (a0-m0-c2k) with a nucleophilic agent, formaldehyde, and a basic catalyst to obtain a compound represented by Formula (a0-m0-c1k)

Hereinafter, each of the steps will be described.

<Step (i)>

In the step (i), a compound represented by Formula (a0-m0-c3k) is reacted with a radical initiator and a halogenating agent to obtain a compound represented by Formula (a0-m0-c2k).

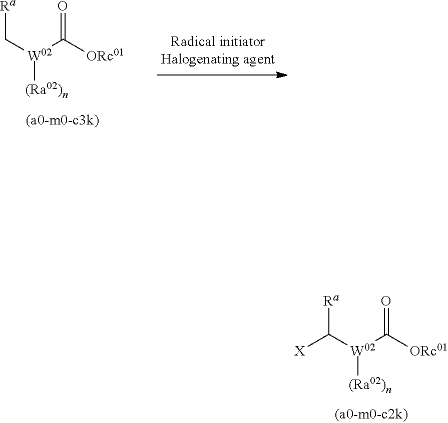

[In the formula, W⁰² represents an aromatic hydrocarbon group which may have a substituent. Rc⁰¹ represents an alkyl group. R^{α} represents a hydrogen atom, a methyl group, or a trifluoromethyl group. Ra⁰² represents an iodine atom or a bromine atom. X represents a halogen atom, n represents an integer of 1 or greater.]

The reaction temperature condition in the step (i) is not particularly limited, and is, for example, about 0° C. to 120° C.

The reaction time of the step (i) is not particularly limited, and is, for example, about 1 to 72 hours.

Examples of the reaction solvent used in the step (i) include tetrachloromethane, dichloromethane, dichloroethane, chloroform, tetrahydrofuran. N,N-dimethylformamide, acetonitrile, propionitrile. N,N′-dimethylacetamide, and dimethyl sulfoxide.

Examples of the radical initiator used in the step (i) include azo-based radical reaction initiators such as 2,2′-azobis(isobutyronitrile), 2,2′-azobis(2-methylbutyronitrile), 2.2′-azobis(2,4-dimethoxyvaleronitrile), and 2,2′-azobis(4-dimethoxy-2.4-dimethylvaleronitrile), and peroxide-based radical initiators such as benzoyl peroxide, dicumyl peroxide, t-butyl peroxybenzoate (BPB), di(4-tert-butylcyclohexyl) peroxydicarbonate (PERKADOX 16), and potassium peroxydisulfate.

Examples of the halogenating agent used in the step (i) include halogenated succinimide and halogenated hydantoin. Examples of the halogen atom in the halogenating agent include a bromine atom, a chlorine atom, an iodine atom, and an iodine atom.

<Step (ii)>

In the step (ii), a compound represented by Formula (a0-m0-c2k), which is obtained by the step (i), is reacted with a nucleophilic agent, formaldehyde, and a basic catalyst to obtain a compound represented by Formula (a0-m0-c1k).

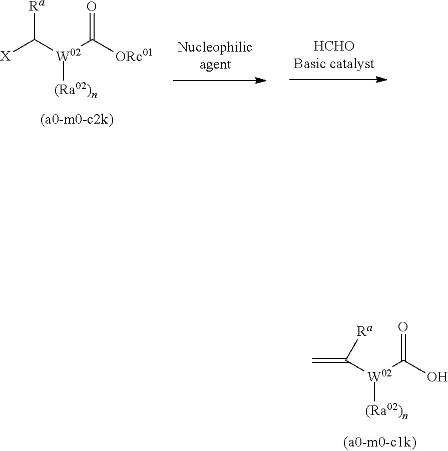

[In the formula, W⁰¹² represents an aromatic hydrocarbon group which may have a substituent. Rc⁰¹ represents an alkyl group. R³ represents a hydrogen atom, a methyl group, or a trifluoromethyl group. Ra⁰² represents an iodine atom or a bromine atom. X represents a halogen atom, n represents an integer of 1 or greater.]

The reaction temperature condition in the step (ii) is not particularly limited, and is, for example, about 0° C. to 150° C.

The reaction time of the step (ii) is not particularly limited, and is, for example, about 1 to 72 hours.

Examples of the reaction solvent used in the step (ii) include toluene, tetrachloromethane, dichloromethane, dichloroethane, chloroform, tetrahydrofuran, N,N-dimethylfornamide, acetonitrile, propionitrile. N,N′-dimethylacetamide, and dimethyl sulfoxide.

Specific examples of the basic catalyst used in the step (ii) include tertiary amines such as potassium tert-butoxide, trimethylamine, triethylamine, and tributylamine; aromatic amines such as pyridine, dimethylaminopyridine, and pyrrolidinopyridine; and diazabicyclononene (DBN), diazabicycloundecene (DBU), and the like.

The compound according to the present embodiment described above is a monomer useful for producing a polymer compound according to a fourth aspect described below.

### (Polymer Compound)

A polymer compound according to the fourth aspect of the present invention has a constitutional unit derived from a compound represented by General Formula (a0-m0).

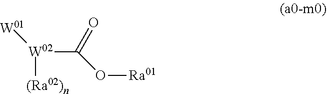

[In the formula, W⁰¹ represents a polymerizable group-containing group. W⁰² represents an aromatic hydrocarbon group which may have a substituent. Ra⁰¹ is an acid dissociable group and represents a group having an alicyclic group, which has no carbon-carbon unsaturated bond, a group having a carbon-carbon unsaturated bond, or a group having an aromatic group. The alicyclic group and the aromatic group may each have a substituent. Ra⁰² represents an iodine atom or a bromine atom, n represents an integer of 1 or greater.]

Suitable examples of the polymer compound according to the fourth aspect of the present invention include a polymer compound having a repeating structure of the constitutional unit (a0) and the constitutional unit (a10) described above. Such a polymer compound is useful as a base material component of the resist composition.

### EXAMPLES

Hereinafter, the present invention will be described with reference to Examples, but the present invention is not limited to the following Examples.

### Synthesis Example of Compound

The compound (a0-m1-1) to the compound (a0-m1-11) and the compound (a0-m2-1) to the compound (a0-m2-8) were each produced by the synthesis method shown below.

[Synthesis of Compound (a0-m1-1)]

10.0 g of methyl 2-iodo-5-methylbenzoate was dissolved in 100 g of terachloromethane, 6.5 g of N-bromosuccinimide (NBS) and 150 mg of azobisisobutyronitrile (AIBN) were added to the solution, and the mixture was stirred at 80° C. under reflux for 4 hours. The solvent was distilled off, and then 6.4 g of methyl 2-iodo-5-bromomethyl benzoate was obtained by performing purification with column chromatography (yield: 50%).

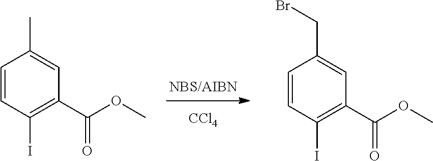

4.1 g of triphenylphosphine and 100 g of toluene was added to 5.0 g of methyl 2-iodo-5-bromomethyl benzoate, and the mixture was stirred at 110° C. under reflux for hours.

The precipitated solid was collected by filtration, 1.7 g of a 37% formaldehyde aqueous solution, 4.0 g of potassium tert-butoxide, 50 g of THF, and 50 g of water were added thereto, and the mixture was stirred at room temperature for 2 hours and then stirred under reflux at 70° C. for 4 hours. The aqueous layer was washed with 50 g of toluene, and citric acid was added thereto. The precipitated solid was collected by filtration, and then crystallization purification was performed to obtain 2-iodo-5-vinylbenzoic acid 2.7 g (yield: 70%).

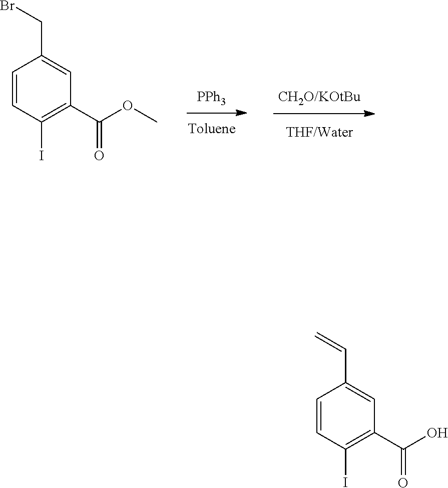

5.5 g of 2-iodo-5-vinylbenzoic acid was dissolved in 40 g of dimethylformamide (DMF), and the solution was added dropwise to a mixed solution of 40 g of DMF, 3.6 g of diazabicycloundecene (DBU), 4.7 g of methylcyclopentanol, and 3.9 g of carbonyldiimidazole (CDT) under ice-cooling. The mixture was stirred at 60° C. for 4 hours, 80 g of TBME was added thereto, and the mixture was washed with water. The solvent was distilled off, and then 5.3 g of the compound (a0-m1-1) was obtained by performing purification by column chromatography (yield: 75%).

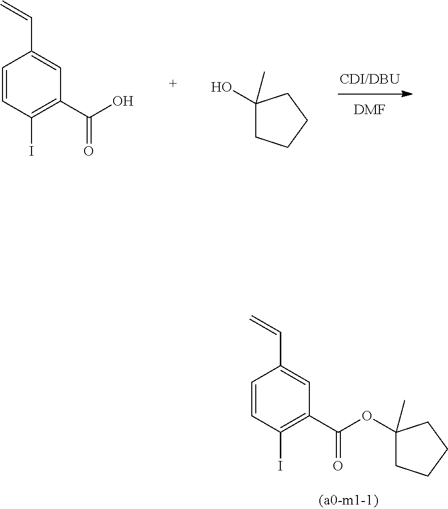

The obtained compound (a0-m1-1) was subjected to NMR measurement, and the structure thereof was identified based on the following data.

1H-NMR (dmso-d6, 400 MHz): δ (ppm)=7.8-8.0 (m, Ar, 3H), 6.7 (dd, CH═CH2, 1H), 5.7 (d, CH═CH2, 1 H), 5.2 (d, CH═CH2, 1H), 2.2 (m, CH2, 2H), 1.6-1.8 (m, CH2, 6H), 1.6 (s, CH3, 3H)

[Synthesis of Compound (a0-m1-2) to Compound (a0-m1-11) and Compound (a0-m2-1) to Compound (a0-m2-8)]

The compound (a0-m1-2) to the compound (a0-m1-11) and the compound (a(m2-1) to the compound (a0-m2-8) were each obtained by the same synthesis method as in the [Synthesis of compound (a0-m1-1)] using the corresponding raw material alcohol.

The structures of the obtained compound (a0-m1-2) to the compound (a0-m1-11) and the compound (a0-m2-1) to the compound (a0-m2-8), and the data of NMR measurements thereof are shown below.

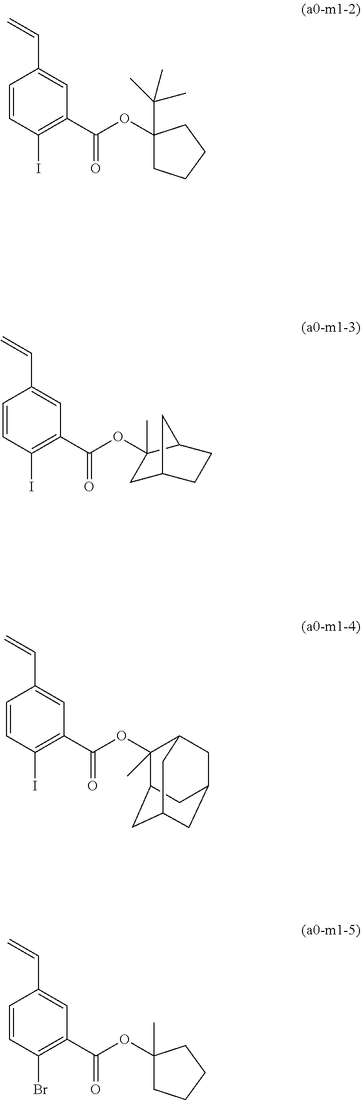 [SMILES: C=Cc1ccc(I)c(C(=O)OC2(C(C)(C)C)CCCC2)c1 ; C=Cc1ccc(I)c(C(=O)OC2(C)CC3CCC2C3)c1 ; C=Cc1ccc(I)c(C(=O)OC2(C)C3CC4CC(C3)CC2C4)c1 ; C=Cc1ccc(Br)c(C(=O)OC2(C)CCCC2)c1]

The obtained compound (a0-m1-2) was subjected to NMR measurement, and the structure thereof was identified based on the following data.

1H-NMR (dmso-d6, 400 MHz): δ (ppm)=7.8-8.0 (m, Ar, 3H), 6.7 (dd, CH═CH2, 1H), 5.7 (d, CH═CH2, 1H), 5.2 (d, CH═CH2, 1H), 2.1-2.2 (m, CH2, 4H), 1.9 (m, CH2, 2H), 1.5 (m, CH2, 2H), 1.0 (s. CH3, 9H)

The obtained compound (a0-m1-3) was subjected to NMR measurement, and the structure thereof was identified based on the following data.

1H-NMR (dmso-d6, 400 MHz): δ (ppm)=7.8-8.0 (m, Ar, 3H), 6.7 (dd, CH═CH2, 1H), 5.7 (d, CH═CH2, 1H), 5.2 (d, CH═CH2, 1H), 2.5 (m, CH, 1H), 2.2 (m, CH, 1H), 1.8 (s, CH3, 3H), 1.1-1.7 (m, CH2, 8H), 1.5 (s, CH3, 3H)

The obtained compound (a0-m1-4) was subjected to NMR measurement, and the structure thereof was identified based on the following data.

1H-NMR (dmso-d6, 400 MHz): δ (ppm)=7.8-8.0 (m, Ar, 3H), 6.7 (dd, CH═CH2, 1H), 5.7 (d, CH═CH2, 1H), 5.2 (d, CH═CH2, 1 H), 2.3 (m, CH2, 2H), 1.5-2.1 (m, CH2, 12H), 1.6 (s, CH3, 3H)

The obtained compound (a0-m1-5) was subjected to NMR measurement, and the structure thereof was identified based on the following data.

1H-NMR (dmso-d6, 400 MHz): δ (ppm)=7.8 (m, Ar, 2H), 7.4 (m, Ar, 1H), 6.7 (dd, CH═CH2, 1H), 5.7 (d, CH═CH2, 1H), 5.2 (d, CH═CH2, 1H), 2.2 (m, CH2, 2H), 1.6-1.8 (m, CH2, 6H), 1.6 (s. CH3, 3H)

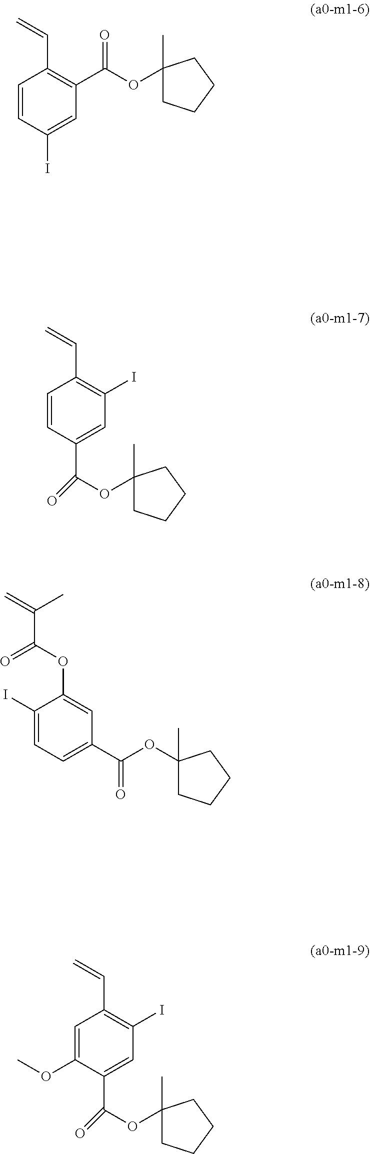 [SMILES: C=Cc1ccc(I)cc1C(=O)OC1(C)CCCC1 ; C=Cc1ccc(C(=O)OC2(C)CCCC2)cc1I ; C=C(C)C(=O)Oc1cc(C(=O)OC2(C)CCCC2)ccc1I ; C=Cc1cc(OC)c(C(=O)OC2(C)CCCC2)cc1I]

The obtained compound (a0-m1-6) was subjected to NMR measurement, and the structure thereof was identified based on the following data.

1H-NMR (dmso-d6, 400 MHz): δ (ppm)=8.0-8.2 (m, Ar, 2H), 7.2 (m, Ar, 1H), 6.7 (dd, CH═CH2, 1H), 5.7 (d, CH═CH2, 1H), 5.2 (d, CH═CH2, 1H), 2.2 (m, CH2, 2H), 1.6-1.8 (m, CH2, 6H), 1.6 (s, CH3, 3H)

The obtained compound (a0-m1-7) was subjected to NMR measurement, and the structure thereof was identified based on the following data.

1H-NMR (dmso-d6, 400 MHz): δ (ppm)=8.0-8.2 (m, Ar, 2H), 7.2 (in. Ar, 1H), 6.7 (dd. CH═CH2, 1H), 5.7 (d, CH═CH2, 1H), 5.2 (d, CH═CH2, 1H), 2.2 (m, CH2, 2H), 1.6-1.8 (m, CH2, 6H), 1.6 (s, CH3, 3H)

The obtained compound (a0-m1-8) was subjected to NMR measurement, and the structure thereof was identified based on the following data.

1H-NMR (dmso-d6, 400 MHz): δ (ppm)=7.9 (m, Ar, 1H), 7.6-7.7 (in. Ar, 2H), 6.0 (s, C═CH2, 1H), 5.6 (s. C═CH2, 1H), 2.2 (m, CH2, 2H), 1.6-1.8 (m, CH2, 6H), 1.9 (s, CH3, 3H), 1.6 (s, CH3, 3H)

The obtained compound (a0-m1-9) was subjected to NMR measurement, and the structure thereof was identified based on the following data.

1H-NMR (dmso-d6, 400 MHz): δ (ppm)=8.1 (m, Ar, 1H), 7.1 (m, Ar, 1H), 6.7 (dd, CH═CH2, 1H), 5.7 (d, CH═CH2, 1H), 5.2 (d, CH═CH2, 1 H), 3.8 (s, OCH3, 3H), 2.2 (m, CH2, 2H), 1.6-1.8 (m, CH2, 6H), 1.6 (s, CH3, 3H)

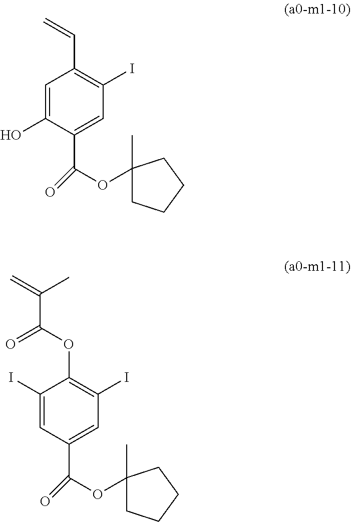 [SMILES: C=Cc1cc(O)c(C(=O)OC2(C)CCCC2)cc1I ; C=C(C)C(=O)Oc1c(I)cc(C(=O)OC2(C)CCCC2)cc1I]

The obtained compound (a0-m1-10) was subjected to NMR measurement, and the structure thereof was identified based on the following data.

1H-NMR (dmso-d6, 400 MHz): δ (ppm)=10.7 (s, OH, 1H), 8.2 (m, Ar, 1H), 7.0 (m, Ar, 1H), 6.7 (dd, CH═CH2, 1H), 5.7 (d, CH═CH2, 1H), 5.2 (d, CH═CH2, 1 H), 2.2 (n, CH2, 2H), 1.6-1.8 (m, CH2, 6H), 1.6 (s, CH3, 3H)

The obtained compound (a0-m1-]1) was subjected to NMR measurement, and the structure thereof was identified based on the following data.

1H-NMR (dmso-d6, 400 MHz): δ (ppm)=8.2 (m, Ar, 2H), 6.0 (s, C═CH2, 1 H), 5.6 (s. C═CH2, 1 H), 2.2 (m, CH2, 2H), 1.6-1.8 (m, CH2, 6H), 1.9 (s, CH3, 3H), 1.6 (s, CH3, 3H)

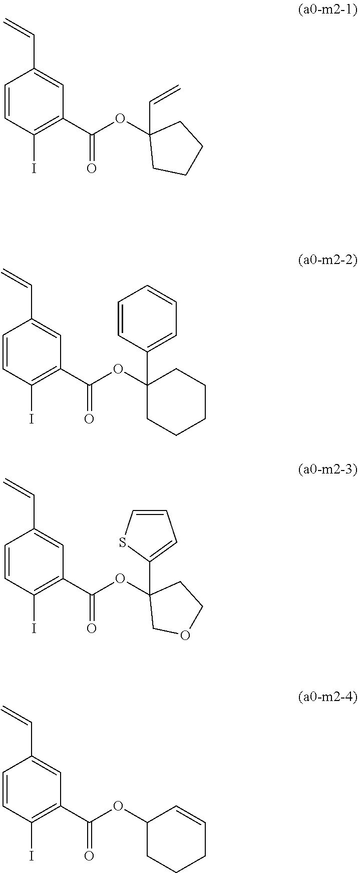 [SMILES: C=Cc1ccc(I)c(C(=O)OC2(C=C)CCCC2)c1 ; C=Cc1ccc(I)c(C(=O)OC2(c3ccccc3)CCCCC2)c1 ; C=Cc1ccc(I)c(C(=O)OC2(c3cccs3)CCOC2)c1 ; C=Cc1ccc(I)c(C(=O)OC2C=CCCC2)c1]

The obtained compound (a0-m2-1) was subjected to NMR measurement, and the structure thereof was identified based on the following data.

1H-NMR (dmso-d6, 400 MHz): δ (ppm)=7.8-8.0 (m, Ar, 3H), 6.7 (dd, CH═CH2, 1H), 6.2 (dd, CH═CH2, 1H), 5.7 (d, CH═CH2, 1H), 5.0-5.2 (m, CH═CH2, 3H), 2.2 (m, CH2, 2H), 1.6-2.0 (m, CH2, 6H)

The obtained compound (a0-m2-2) was subjected to NMR measurement, and the structure thereof was identified based on the following data.

1H-NMR (dmso-d6, 400 MHz): δ (ppm)=7.8-8.0 (m, Ar, 3H), 7.2-7.4 (m, Ph, 5H), 6.7 (dd, CH═CH2, 1H), 5.7 (d, CH═CH2, 1H), 5.2 (d, CH═CH2, 1H), 2.2 (m, CH2, 2H), 1.5-1.8 (m, CH2, 7H), 1.3 (m, CH2, 1H)

The obtained compound (a0-m2-3) was subjected to NMR measurement, and the structure thereof was identified based on the following data.

1H-NMR (dmso-d6, 400 MHz): δ (ppm)=7.8-8.0 (m, Ar, 3H), 7.5 (s, thiophene, 1H), 7.1 (s, thiophene, 1H), 7.0 (s, thiophene, 1H), 6.7 (dd, CH═CH2, 1H), 5.7 (d, CH═CH2, 1H), 5.2 (d, CH═CH2, 1H), 4.3 (m, CH2, 1H), 4.1 (m, CH2, 1H), 3.9 (m, CH2, 2H), 2.7 (m, CH2, 1H), 2.5 (m, CH2, 1H)

The obtained compound (a0-m2-4) was subjected to NMR measurement, and the structure thereof was identified based on the following data.

1H-NMR (dmso-d6, 400 MHz): δ (ppm)=7.8-8.0 (m, Ar, 3H), 6.7 (dd, CH—=CH2, 1H), 6.1 (m, CH, 1H), 5.8 (m, CH, 1H), 5.7 (d, CH═CH2, 1H), 5.5 (m, CH, 1H), 5.2 (d, CH═CH2, 1H), 1.6-2.1 (m, CH2, 6H)

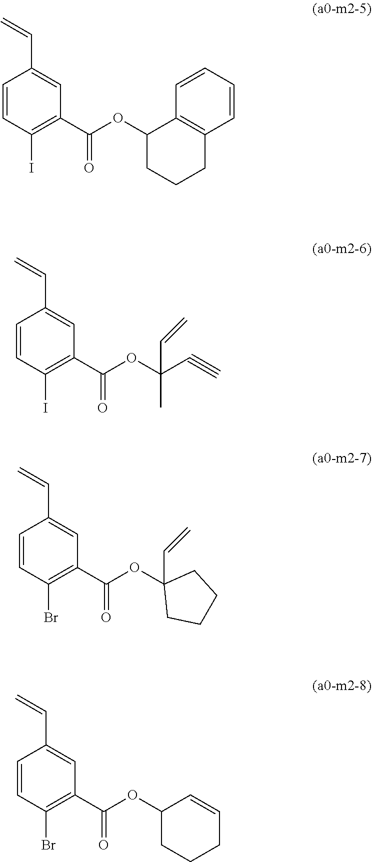 [SMILES: C=Cc1ccc(I)c(C(=O)OC2CCCc3ccccc32)c1 ; C#CC(C)(C=C)OC(=O)c1cc(C=C)ccc1I ; C=Cc1ccc(Br)c(C(=O)OC2(C=C)CCCC2)c1 ; C=Cc1ccc(Br)c(C(=O)OC2C=CCCC2)c1]

The obtained compound (a0-m2-5) was subjected to NMR measurement, and the structure thereof was identified based on the following data.

1H-NMR (dmso-d6, 400 MHz): δ (ppm)=7.8-8.0 (m, Ar, 3H), 7.0-7.4 (m, Ar, 4H), 6.7 (dd, CH—CH2, 1H), 5.9 (m, CH, 1H), 5.7 (d, CH—CH2, 1H), 5.2 (d, CH—CH2, 1H), 2.7 (m, CH2, 1H), 2.5 (m, CH2, 1H), 1.7-2.0 (m, CH2, 4H)

The obtained compound (a0-m2-6) was subjected to NMR measurement, and the structure thereof was identified based on the following data.

1H-NMR (dmso-d6. 400 MHz): δ (ppm)=7.8-8.0 (m, Ar, 3H), 6.7 (dd. CH═CH2, 1H), 6.0 (dd, CH═CH2, 1H), 5.7 (d, CH═CH2, 1H), 5.5 (d, CH═CH2, 1H), 5.2 (m, CH═CH2, 2H), 3.7 (s, CH, 1H), 1.7 (s, CH3, 3H)

The obtained compound (a0-m2-7) was subjected to NMR measurement, and the structure thereof was identified based on the following data.

1H-NMR (dmso-d6, 400 MHz): δ (ppm)=7.8 (m, Ar, 2H), 7.4 (m, Ar, 1H), 6.7 (dd, CH═CH2, 1H), 6.2 (dd, CH═CH2, 1H), 5.7 (d, CH═CH2, 1H), 5.0-5.2 (m, CH═CH2, 3H), 2.2 (m, CH2, 2H), 1.6-2.0 (m, CH2, 6H)

The obtained compound (a0-m2-8) was subjected to NMR measurement, and the structure thereof was identified based on the following data.

1H-NMR (dmso-d6, 400 MHz): δ (ppm)=7.8 (m, Ar, 2H), 7.4 (m, Ar, 1H), 6.7 (dd, CH═CH2, 1H), 6.1 (m, CH, 1H), 5.8 (m, CH, 1H), 5.7 (d, CH═CH2, 1H), 5.5 (in. CH, 1H), 5.2 (m, CH═CH2, 2H), 1.6-2.1 (m, CH2, 6H)

### Synthesis Example of Polymer Compound

Using the compound (a0-m I-1) to the compound (a0-m1-1), the compound (a0-m2-1) to the compound (a0-m2-8), the following compound (a10-1pre), compound (a10-2pre), compound (a10-3pre), and compound (a10-4), the compound (a2-1) to the compound (a2-2), and the compound (a1-1) to the compound (a1-5), each of the polymer compound (A1-1) to the polymer compound (A1-25) and the polymer compound (A2-1) to the compound (A2-3) was produced by the following synthesis method.

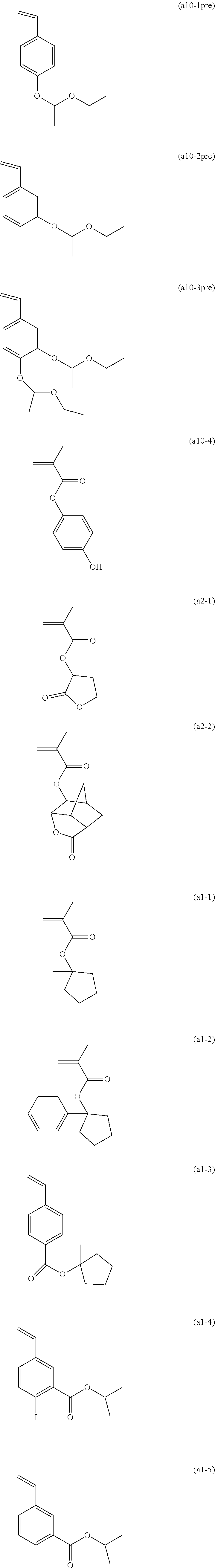

### [Synthesis of Polymer Compound (A1-1)]

7.1 g of the compound (a0-m1-1), 3.8 g of the compound (a10-1pre), and 0.8 g of dimethyl azobis(isobutyrate) (V-601) as a polymerization initiator were dissolved in g of methyl ethyl ketone (MEK), and the solution was heated to 70° C. under a nitrogen atmosphere and stirred for 5 hours.

Thereafter, 2.0 g of acetic acid and 60 g of methanol (MeOH) were added to the reaction solution, and the deprotection reaction was carried out at 30° C. for 18 hours. After completion of the reaction, the obtained reaction solution was precipitated in 600 g of heptane, followed by washing. The obtained white solid was filtered and then subjected to reduced pressure drying overnight to obtain 6.4 g of a polymer compound (A1-1) as a target substance. The structure of the obtained polymer compounds is shown below.

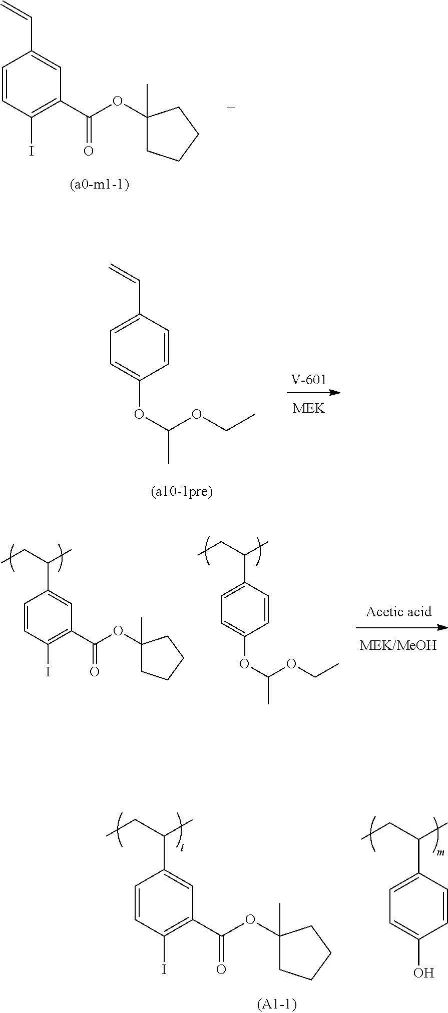

The weight-average molecular weight (Mw) of the obtained polymer compound (A1-1) in terms of standard polystyrene, determined by GPC measurement, was 5800, and the polydispersity (Mw/Mn) thereof was 1.59.

Further, the copolymer compositional ratio (ratio (molar ratio) between constitutional units in the structural formula) determined from the carbon 13 nuclear magnetic resonance spectrum (150 MHZ ¹³C-NMR) was 1/m=50/50.

### [Synthesis of Polymer Compound (A1-2) to Polymer Compound (A1-12), Polymer Compound (A1-14) to Polymer Compound (A1-25), and Polymer Compound (A2-1) to Polymer Compound (A2-3)]

The polymer compound (A1-2) to the polymer compound (A1-12), the polymer compound (A1-14) to the polymer compound (A1-25), and the polymer compound (A2-1) to the polymer compound (A2-3) were each obtained by the same synthesis method (radical polymerization and deprotection reaction) as in [Synthesis of polymer compound (A1-1)] using the corresponding raw material monomers. The structure of each of the obtained polymer compounds is shown below.

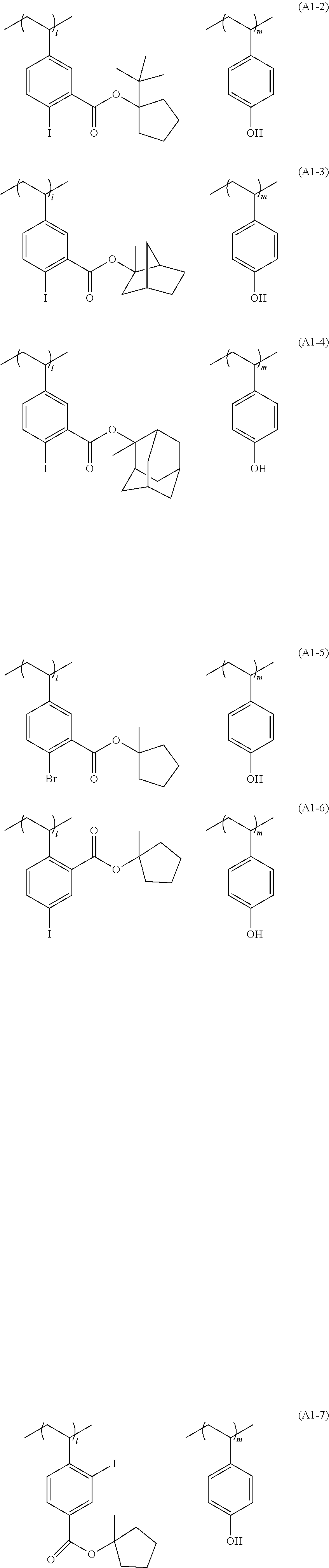

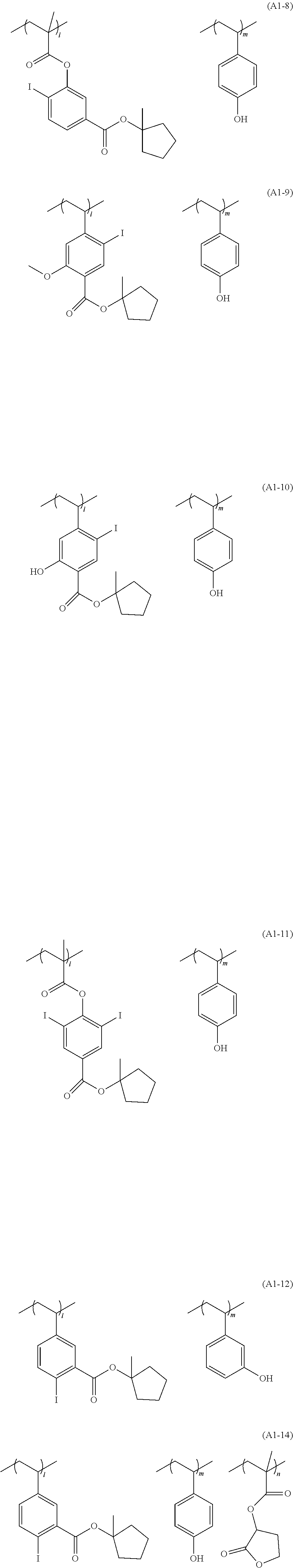

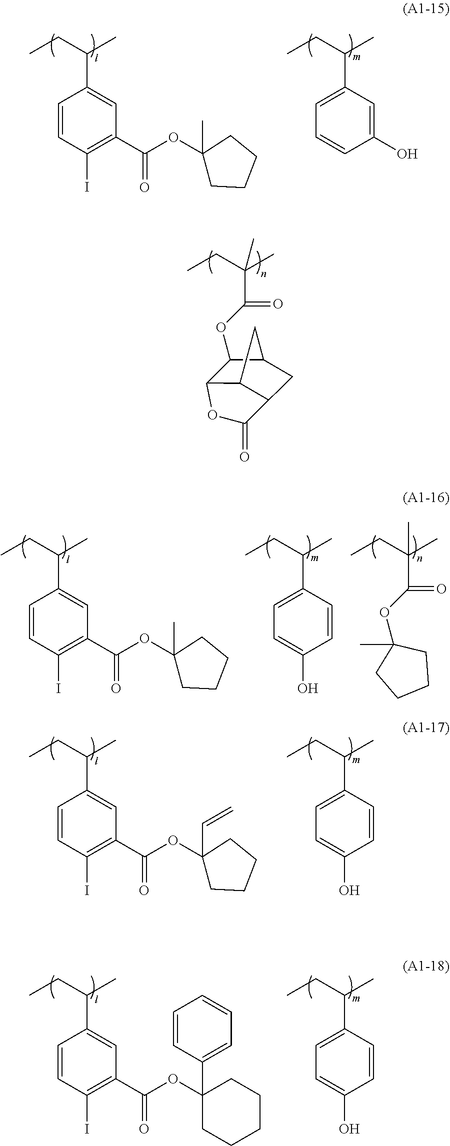

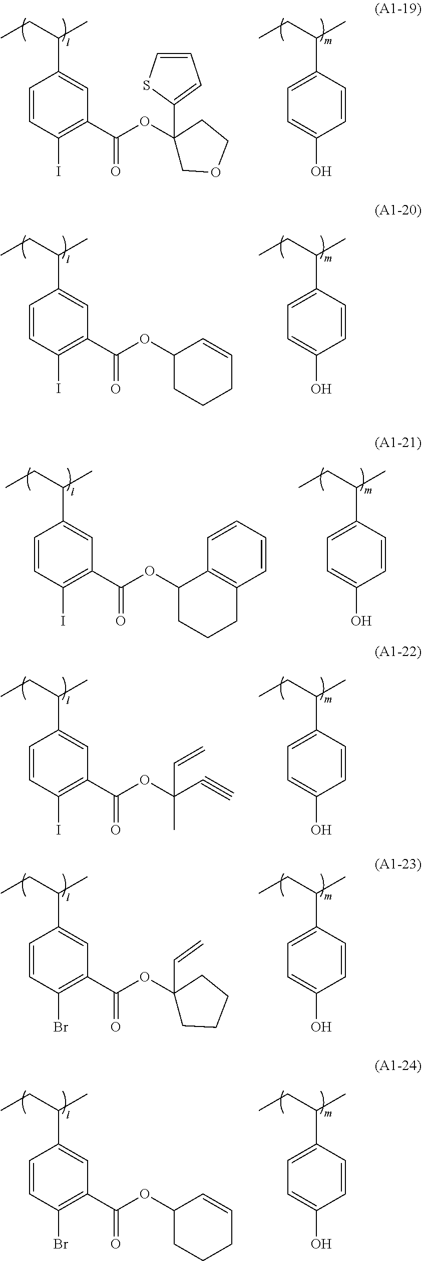

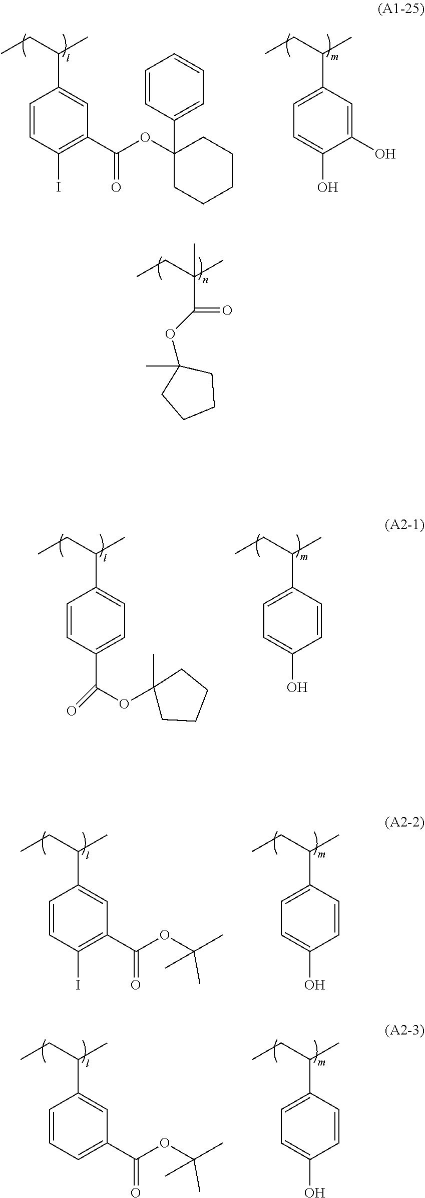

With respect to each of the obtained polymer compounds, the copolymer compositional ratio (the ratio (molar ratio) between constitutional units derived from compounds which are each monomers) of the polymer compound, determined by ¹³C-NMR, and the weight-average molecular weight (Mw) and the polydispersity (Mw/Mn) in terms of standard polystyrene, determined by GPC measurement, are collectively listed in Tables 1 and 2.

### [Synthesis of Polymer Compound (A1-13)]

10.0 g of the compound (a0-m1-1), 3.3 g of the compound (a10-4), and 0.9 g of dimethyl azobis(isobutyrate) (V-601) as a polymerization initiator were dissolved in 30 g of methyl ethyl ketone (MEK), and the solution was heated to 70° C. under a nitrogen atmosphere and stirred for 5 hours.

Thereafter, the reaction solution was precipitated in 400 g of heptane, and the obtained white solid was washed with 200 g of heptane. The obtained white solid was filtered and then subjected to reduced pressure drying overnight to obtain 7.5 g of a polymer compound (A1-13) as a target substance. The structure of the obtained polymer compounds is shown below.

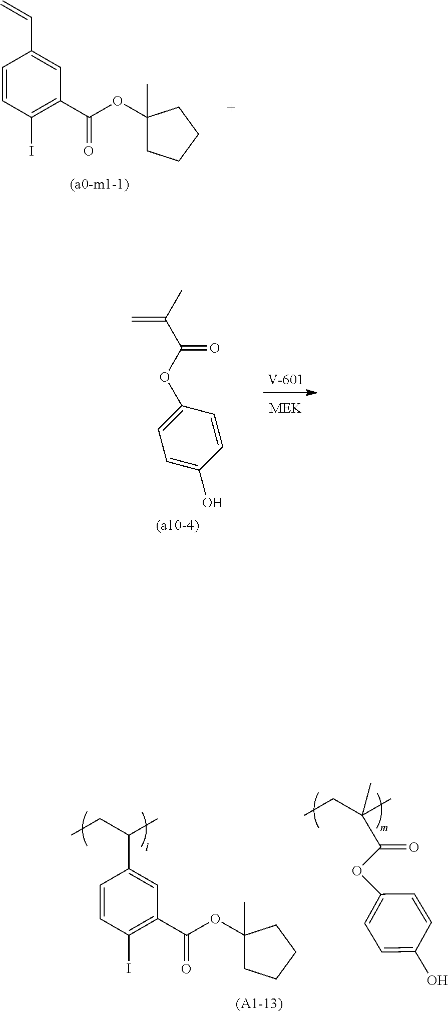

The weight-average molecular weight (Mw) of the obtained polymer compound (A1-13) in terms of standard polystyrene, determined by GPC measurement, was 5500, and the polydispersity (Mw/Mn) thereof was 1.67.

Further, the copolymer compositional ratio (ratio (molar ratio) between constitutional units in the structural formula) determined from the carbon 13 nuclear magnetic resonance spectrum (150 MHZ ¹³C-NMR) was 1/m=60/40.

TABLE 1

| | | Copolymer | Weight-average | |
|---|---|---|---|---|
| Polymer | Composition of raw | compositional | molecular weight | Polydispersity |
| compound | material monomer | ratio | (Mw) | (Mw/Mn) |
| (A1-1) | (a0-m1-1)/(a10-1pre) | l/m = 50/50 | 5800 | 1.59 |
| (A1-2) | (a0-m1-2)/(a10-1pre) | l/m = 50/50 | 6200 | 1.61 |
| (A1-3) | (a0-m1-3)/(a10-1pre) | l/m = 50/50 | 6100 | 1.62 |
| (A1-4) | (a0-m1-4)/(a10-1pre) | l/m = 50/50 | 7100 | 1.62 |
| (A1-5) | (a0-m1-5)/(a10-1pre) | l/m = 50/50 | 6100 | 1.65 |
| (A1-6) | (a0-m1-6)/(a10-1pre) | l/m = 50/50 | 7200 | 1.63 |
| (A1-7) | (a0-m1-7)/(a10-1pre) | l/m = 50/50 | 7400 | 1.61 |
| (A1-8) | (a0-m1-8)/(a10-1pre) | l/m = 50/50 | 6500 | 1.53 |
| (A1-9) | (a0-m1-9)/(a10-1pre) | l/m = 50/50 | 7100 | 1.62 |
| (A1-10) | (a0-m1-10)/(a10-1pre) | l/m = 50/50 | 6900 | 1.64 |
| (A1-11) | (a0-m1-11)/(a10-1pre) | l/m = 50/50 | 6400 | 1.65 |
| (A1-12) | (a0-m1-1)/(a10-2pre) | l/m = 50/50 | 6200 | 1.58 |
| (A1-13) | (a0-m1-1)/(a10-4) | l/m = 60/40 | 5500 | 1.67 |
| (A1-14) | (a0-m1-1)/(a10-1pre)/(a2-1) | l/m/n = 50/40/10 | 6300 | 1.62 |
| (A1-15) | (a0-m1-1)/(a10-2pre)/(a2-2) | l/m/n = 50/40/10 | 5700 | 1.63 |
| (A1-16) | (a0-m1-1)/(a10-1pre)/(a1-1) | l/m/n = 40/40/20 | 6400 | 1.58 |

TABLE 2

| | | Copolymer | Weight-average | |
|---|---|---|---|---|
| Polymer | Composition of raw | compositional | molecular weight | Polydispersity |
| compound | material monomer | ratio | (Mw) | (Mw/Mn) |
| (A1-17) | (a0-m2-1)/(a10-1pre) | l/m = 50/50 | 5700 | 1.63 |
| (A1-18) | (a0-m2-2)/(a10-1pre) | l/m = 50/50 | 6400 | 1.60 |
| (A1-19) | (a0-m2-3)/(a10-1pre) | l/m = 50/50 | 5500 | 1.58 |
| (A1-20) | (a0-m2-4)/(a10-1pre) | l/m = 50/50 | 5400 | 1.61 |
| (A1-21) | (a0-m2-5)/(a10-1pre) | l/m = 50/50 | 6500 | 1.62 |
| (A1-22) | (a0-m2-6)/(a10-1pre) | l/m = 50/50 | 5700 | 1.55 |
| (A1-23) | (a0-m2-7)/(a10-1pre) | l/m = 50/50 | 6100 | 1.62 |
| (A1-24) | (a0-m2-8)/(a10-1pre) | l/m = 50/50 | 5700 | 1.59 |
| (A1-25) | (a0-m2-2)/(a10-3pre)/(a1-2) | l/m/n = 50/30/20 | 7400 | 1.65 |
| (A2-1) | (a1-3)/(a10-1pre) | l/m = 50/50 | 6500 | 1.62 |
| (A2-2) | (a1-4)/(a10-1pre) | l/m = 50/50 | 6600 | 1.66 |
| (A2-3) | (a1-5)/(a10-1pre) | l/m = 50/50 | 6900 | 1.63 |

### <Preparation of Resist Composition>

### Examples 1 to 34 and Comparative Examples 1 to 3

Each of the components shown in Tables 3 and 5 was mixed and dissolved to prepare a resist composition of each Example.

TABLE 3

| Resist composition | Component (A) | Component (B) | Component (D) | Component (S) |
|---|---|---|---|---|
| Example 1 | (A)-1 | (B)-1 | (D)-1 | (S)-1 |
| | [100] | [25.0] | [6.0] | [6400] |
| Example 2 | (A)-2 | (B)-1 | (D)-1 | (S)-1 |
| | [100] | [25.0] | [6.0] | [6400] |
| Example 3 | (A)-3 | (B)-1 | (D)-1 | (S)-1 |
| | [100] | [25.0] | [6.0] | [6400] |
| Example 4 | (A)-4 | (B)-1 | (D)-1 | (S)-1 |
| | [100] | [25.0] | [6.0] | [6400] |
| Example 5 | (A)-5 | (B)-1 | (D)-1 | (S)-1 |
| | [100] | [25.0] | [6.0] | [6400] |
| Example 6 | (A)-6 | (B)-1 | (D)-1 | (S)-1 |
| | [100] | [25.0] | [6.0] | [6400] |
| Example 7 | (A)-7 | (B)-1 | (D)-1 | (S)-1 |
| | [100] | [25.0] | [6.0] | [6400] |
| Example 8 | (A)-8 | (B)-1 | (D)-1 | (S)-1 |
| | [100] | [25.0] | [6.0] | [6400] |
| Example 9 | (A)-9 | (B)-1 | (D)-1 | (S)-1 |
| | [100] | [25.0] | [6.0] | [6400] |
| Example 10 | (A)-10 | (B)-1 | (D)-1 | (S)-1 |
| | [100] | [25.0] | [6.0] | [6400] |
| Example 11 | (A)-11 | (B)-1 | (D)-1 | (S)-1 |
| | [100] | [25.0] | [6.0] | [6400] |
| Example 12 | (A)-12 | (B)-1 | (D)-1 | (S)-1 |
| | [100] | [25.0] | [6.0] | [6400] |
| Example 13 | (A)-13 | (B)-1 | (D)-1 | (S)-1 |
| | [100] | [25.0] | [6.0] | [6400] |
| Example 14 | (A)-14 | (B)-1 | (D)-1 | (S)-1 |
| | [100] | [25.0] | [6.0] | [6400] |
| Example 15 | (A)-15 | (B)-1 | (D)-1 | (S)-1 |
| | [100] | [25.0] | [6.0] | [6400] |
| Example 16 | (A)-16 | (B)-1 | (D)-1 | (S)-1 |
| | [100] | [25.0] | [6.0] | [6400] |

TABLE 4

| Resist composition | Component (A) | Component (B) | Component (D) | Component (S) |
|---|---|---|---|---|
| Example 17 | (A)-17 | (B)-1 | (D)-1 | (S)-1 |
| | [100] | [25.0] | [6.0] | [6400] |
| Example 18 | (A)-18 | (B)-1 | (D)-1 | (S)-1 |
| | [100] | [25.0] | [6.0] | [6400] |
| Example 19 | (A)-19 | (B)-1 | (D)-1 | (S)-1 |
| | [100] | [25.0] | [6.0] | [6400] |
| Example 20 | (A)-20 | (B)-1 | (D)-1 | (S)-1 |
| | [100] | [25.0] | [6.0] | [6400] |
| Example 21 | (A)-21 | (B)-1 | (D)-1 | (S)-1 |
| | [100] | [25.0] | [6.0] | [6400] |
| Example 22 | (A)-22 | (B)-1 | (D)-1 | (S)-1 |
| | [100] | [25.0] | [6.0] | [6400] |
| Example 23 | (A)-23 | (B)-1 | (D)-1 | (S)-1 |
| | [100] | [25.0] | [6.0] | [6400] |
| Example 24 | (A)-24 | (B)-1 | (D)-1 | (S)-1 |
| | [100] | [25.0] | [6.0] | [6400] |
| Example 25 | (A)-25 | (B)-1 | (D)-1 | (S)-1 |
| | [100] | [25.0] | [6.0] | [6400] |

TABLE 5

| Resist composition | Component (A) | Component (B) | Component (D) | Component (S) |
|---|---|---|---|---|
| Example 26 | (A)-1 | (B)-2 | (D)-1 | (S)-1 |
| | [100] | [25.2] | [6.0] | [6400] |
| Example 27 | (A)-1 | (B)-3 | (D)-1 | (S)-1 |
| | [100] | [23.3] | [6.0] | [6400] |
| Example 28 | (A)-1 | (B)-4 | (D)-1 | (S)-1 |
| | [100] | [25.5] | [6.0] | [6400] |
| Example 29 | (A)-1 | (B)-5 | (D)-1 | (S)-1 |
| | [100] | [37.3] | [6.0] | [6400] |
| Example 30 | (A)-1 | (B)-6 | (D)-1 | (S)-1 |
| | [100] | [40.4] | [6.0] | [6400] |
| Example 31 | (A)-1 | (B)-7 | (D)-1 | (S)-1 |
| | [100] | [33.5] | [6.0] | [6400] |
| Example 32 | (A)-1 | (B)-8 | (D)-1 | (S)-1 |
| | [100] | [28.4] | [6.0] | [6400] |
| Example 33 | (A)-1 | (B)-1 | (D)-2 | (S)-1 |
| | [100] | [25.0] | [7.3] | [6400] |
| Example 34 | (A)-1 | (B)-1 | (D)-3 | (S)-1 |
| | [100] | [25.0] | [10.6] | [6400] |
| Comparative | (A)-26 | (B)-1 | (D)-1 | (S)-1 |
| Example 1 | [100] | [25.0] | [6.0] | [6400] |
| Comparative | (A)-27 | (B)-1 | (D)-1 | (S)-1 |
| Example 2 | [100] | [25.0] | [6.0] | [6400] |
| Comparative | (A)-28 | (B)-1 | (D)-1 | (S)-1 |
| Example 3 | [100] | [25.0] | [6.0] | [6400] |

In Tables 3 to 5, each abbreviation has the following meaning. The numerical values in the brackets are blending amounts (parts by mass).
(A)-1 to (A)-25: polymer compounds (A11) to (A1-25) shown above.
(A)-26 to (A)-28: polymer compounds (A2-1) to (A2-3) shown above.
(B)-1: acid generator consisting of compound represented by Chemical Formula (B-1).
(B)-2: acid generator consisting of compound represented by Chemical Formula (B-2).
(B)-3: acid generator consisting of compound represented by Chemical Formula (B-3).
(B)-4: acid generator consisting of a compound represented by Chemical Formula (B-4).
(B)-5: acid generator consisting of a compound represented by Chemical Formula (B-5).
(B)-6: acid generator consisting of a compound represented by Chemical Formula (B-6).
(B)-7: acid generator consisting of a compound represented by Chemical Formula (B-7).
(B)-8: acid generator consisting of a compound represented by Chemical Formula (B-8).

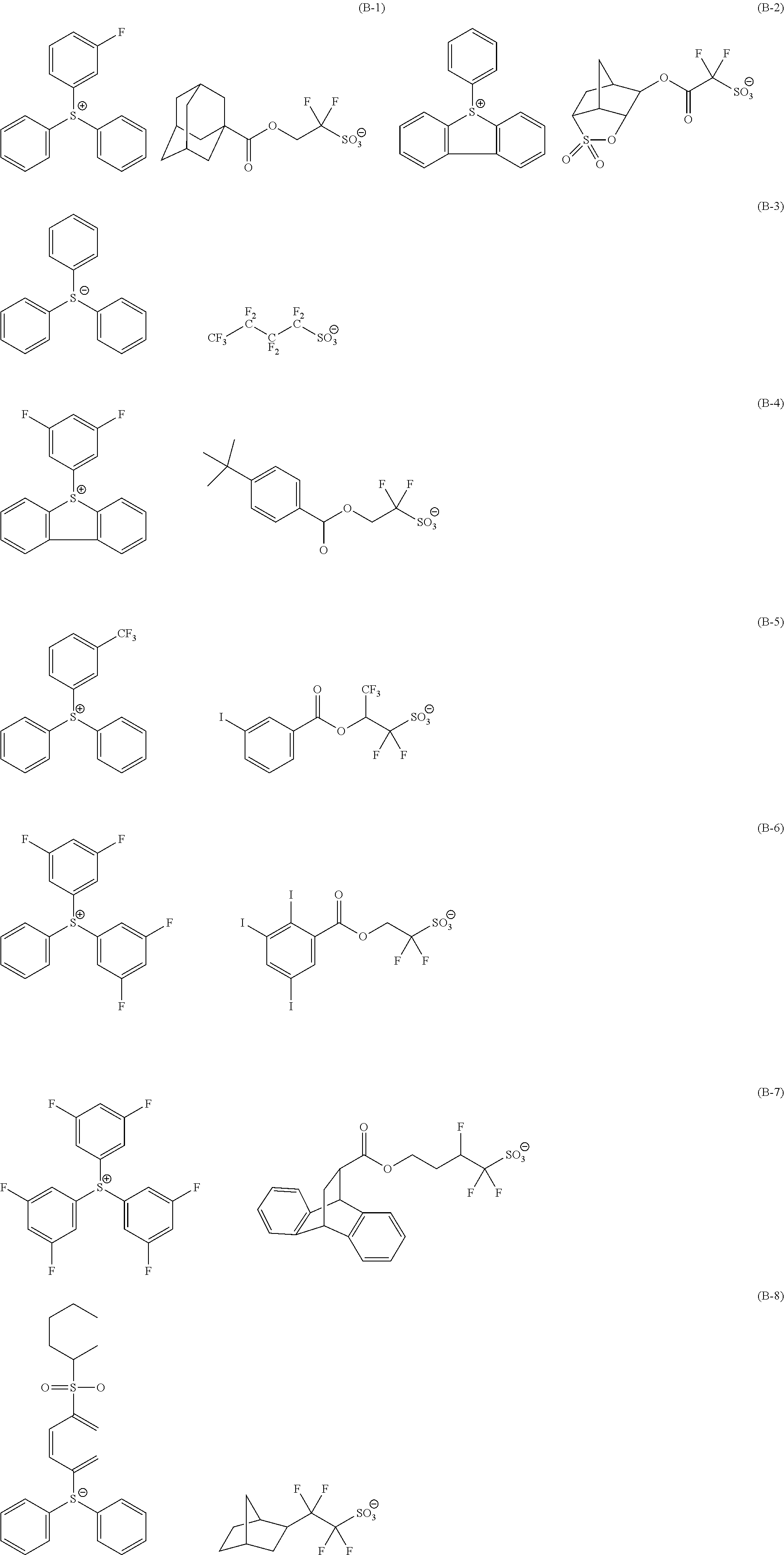

(D)-1: acid diffusion control agent consisting of compound represented by Chemical Formula (D-1).
(D)-2: acid diffusion control agent consisting of compound represented by Chemical Formula (D1-2).
(D)-3: acid diffusion control agent consisting of compound represented by Chemical Formula (D1-3).
(S)-1: mixed solvent of propylene glycol monomethyl ether acetate/propylene glycol monomethyl ether=60/40 (mass ratio).

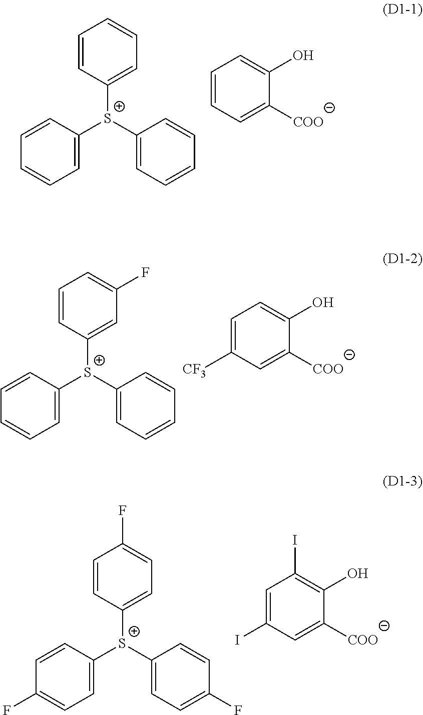

### <Evaluation>

A resist pattern (LS pattern) of a line-and-space pattern was formed by the resist pattern formation method shown below, and sensitivity, line width roughness (LWR), and etching resistance were evaluated.

### <<Formation of Resist Pattern>>

An 8-inch silicon substrate which had been subjected to a hexamethyldisilazane (HMDS) treatment was coated with each resist composition of each example using a spinner, and subjected to a pre-bake (PAB) treatment on a hot plate at a temperature of 110° C. for 60 seconds so that the 8-inch silicon substrate was dried, thereby forming a resist film having a film thickness of 50 nm.

Next, the resist film was drawn (exposed) using an electron beam drawing apparatus JEOL JBX-9300FS (manufactured by JEOL Co., Ltd.) at an acceleration voltage of 100 kV (Beam current: 100 pA, Scan step: 4 nm) such that a target size was a 1:1 line-and-space pattern (hereinafter referred to as an [S pattern) having a line width of 50 nm (pitch width: 100 nm).

Thereafter, a post exposure bake (PEB) treatment was performed thereon at 100° C. for 60 seconds.

Next, using a development device (clean track ACT8, manufactured by Tokyo Electron Co., Ltd.), an aqueous 2.38% by mass tetramethylammonium hydroxide (TMAH) solution “NMD-3” (trade name, manufactured by Tokyo Ohka Kogyo Co., Ltd.) at 23° C. was dropped onto the resist film from an LD-nozzle to perform alkali development for 60 seconds.

Thereafter, water rinsing was carried out with pure water for 15 seconds.

As a result, in any of the examples, each of 1:1 LS patterns having a line width of 50 nm (pitch width of 100 nm) was formed.

### [Evaluation of Sensitivity]

In the above-described <<Formation of resist pattern>>, an optimum exposure amount Eop (μC/cm²) in a case where the LS pattern was formed was obtained. The results are listed in Tables 6 to 8 in the columns of “Bop (μC/cm²”.

### [Evaluation of Line Width Roughness (LWR)]

In the IS pattern formed by the <Fonation of resist pattern>, a space width was measured at 400 points in the longitudinal direction of the space by a length-measuring SEM (scanning electron microscope, acceleration voltage: 300 V, trade name: S-9380, manufactured by Hitachi High-Tech Technologies Corporation), a three times value (3s) of a standard deviation (s) was obtained from the result, and a value obtained by averaging 3s at 400 points was calculated as a measure indicating LWR. The results are shown in Tables 6 to 8 as “LWR (nm)”.

The smaller the value of 3s, the smaller the roughness of the line width, and the more uniform width of the LS pattern was obtained.

### [Evaluation of Etching Resistance]

In the above-described <<Formation of resist pattern>>, with respect to each resist film after the PAB and before the exposure, a dry etching treatment was performed for 60 seconds using a dry etching device TCA-3822 (trade name, manufactured by Tokyo Ohka Kogyo Co., Ltd.) and CF₄ gas.

The residual film amount was determined from the film thicknesses of the resist film before and after the dry etching treatment.

A film thickness after the dry etching treatment was evaluated as “A” in a case where the film thickness after the dry etching treatment was 80% or more with respect to the film thickness before the dry etching treatment, and was evaluated as “B” in a case where the film thickness after the dry etching treatment was less than 80% with respect to the film thickness before the dry etching treatment. The results are listed in the columns of “etching resistance” in Tables 6 to 8.

TABLE 6

| Resist | PAB | PEB | Eop | LWR | Etching |
|---|---|---|---|---|---|
| composition | (° C.) | (° C.) | (μC/cm²) | (nm) | resistance |
| Example 1 | 110 | 100 | 106 | 3.2 | A |
| Example 2 | 110 | 100 | 103 | 3.4 | A |
| Example 3 | 110 | 100 | 112 | 3.5 | A |
| Example 4 | 110 | 100 | 112 | 3.6 | A |
| Example 5 | 110 | 100 | 116 | 3.5 | A |
| Example 6 | 110 | 100 | 112 | 3.2 | A |
| Example 7 | 110 | 100 | 107 | 3.3 | A |
| Example 8 | 110 | 100 | 103 | 3.5 | A |
| Example 9 | 110 | 100 | 100 | 2.9 | A |
| Example 10 | 110 | 100 | 111 | 3.2 | A |
| Example 11 | 110 | 100 | 96 | 2.7 | A |
| Example 12 | 110 | 100 | 104 | 3.1 | A |
| Example 13 | 110 | 100 | 97 | 3.2 | A |
| Example 14 | 110 | 100 | 106 | 3.5 | A |
| Example 15 | 110 | 100 | 112 | 3.2 | A |
| Example 16 | 110 | 100 | 105 | 3.4 | A |

TABLE 7

| Resist | PAB | PEB | Eop | LWR | Etching |
|---|---|---|---|---|---|
| composition | (° C.) | (° C.) | (μC/cm²) | (nm) | resistance |
| Example 17 | 110 | 100 | 102 | 2.9 | A |
| Example 18 | 110 | 100 | 102 | 3.0 | A |
| Example 19 | 110 | 100 | 98 | 3.7 | A |
| Example 20 | 110 | 100 | 109 | 3.2 | A |
| Example 21 | 110 | 100 | 105 | 3.3 | A |
| Example 22 | 110 | 100 | 111 | 3.5 | A |
| Example 23 | 110 | 100 | 112 | 3.3 | A |
| Example 24 | 110 | 100 | 119 | 3.6 | A |
| Example 25 | 110 | 100 | 102 | 3.9 | A |

TABLE 8

| Resist | PAB | PEB | Eop | LWR | Etching |
|---|---|---|---|---|---|
| composition | (° C.) | (° C.) | (μC/cm²) | (nm) | resistance |
| Example 26 | 110 | 100 | 110 | 3.5 | A |
| Example 27 | 110 | 100 | 105 | 3.6 | A |
| Example 28 | 110 | 100 | 103 | 3.0 | A |
| Example 29 | 110 | 100 | 98 | 2.7 | A |
| Example 30 | 110 | 100 | 94 | 2.7 | A |
| Example 31 | 110 | 100 | 100 | 2.9 | A |
| Example 32 | 110 | 100 | 105 | 3.4 | A |
| Example 33 | 110 | 100 | 102 | 3.2 | A |
| Example 34 | 110 | 100 | 95 | 2.8 | A |
| Comparative | 110 | 100 | 136 | 5.4 | A |
| Example 1 | | | | | |
| Comparative | 110 | 100 | 122 | 3.8 | B |
| Example 2 | | | | | |
| Comparative | 110 | 100 | 140 | 5.2 | B |
| Example 3 | | | | | |

From the results shown in Tables 6 to 8, it was confirmed that, in the resist compositions of Examples 1 to 34 to which the present invention was applied, the sensitivity, the roughness characteristic, and the etching resistance were all satisfactory.

On the other hand, in the resist composition of Comparative Example 1, which was out of the scope of the present invention, the sensitivity and the roughness characteristics were not sufficient because W⁰² does not have an iodine atom.

In Comparative Example 2, which is outside the scope of the present invention, the resist composition having Ra⁰¹ not having “group having an alicyclic group, which has no carbon-carbon unsaturated bond, group having a carbon-carbon unsaturated bond, or group having an aromatic group” had insufficient etching resistance.

In Comparative Example 3, which is outside the scope of the present invention, in which W⁰² does not have an iodine atom and Ra⁰¹ does not have “a group having an alicyclic group, which has no carbon-carbon unsaturated bond, a group having a carbon-carbon unsaturated bond, or a group having an aromatic group”, the sensitivity and the roughness characteristic were not sufficient, and the etching resistance was not sufficient.

1. A resist composition that generates an acid upon light exposure and in which solubility in a developing solution is changed by an action of the acid, the resist composition comprising:
a resin component (A1) in which solubility in a developing solution is changed by an action of an acid,
wherein the resin component (A1) has a constitutional unit (a0) derived from a compound represented by General Formula (a0-m0), 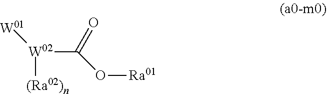
wherein W⁰¹ represents a polymerizable group-containing group, W⁰² represents an aromatic hydrocarbon group which may have a substituent, Ra⁰¹ is an acid dissociable group and represents a group having an alicyclic group, which has no carbon-carbon unsaturated bond, a group having a carbon-carbon unsaturated bond, or a group having an aromatic group, where the alicyclic group and the aromatic group may each have a substituent, Ra⁰² represents an iodine atom or a bromine atom, and n represents an integer of 1 or greater.

2. The resist composition according to claim 1, wherein the constitutional unit (a0) is represented by General Formula (a0-1), 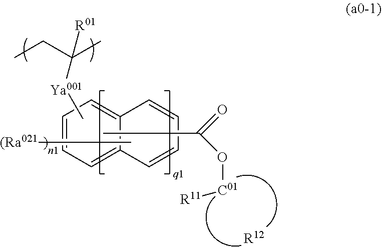
wherein R⁰¹ represents an alkyl group having 1 to 5 carbon atoms, a halogenated alkyl group having 1 to 5 carbon atoms, or a hydrogen atom, Ya⁰⁰¹ represents a divalent linking group or a single bond, C⁰¹ represents a tertiary carbon atom, R″ represents a chain-like saturated hydrocarbon group which may have a substituent, R¹² represents a group forming an alicyclic group with C⁰¹, which has no carbon-carbon unsaturated bond, the alicyclic group here may have a substituent, Ra⁰²¹ represents an iodine atom or a bromine atom, q1 represents an integer of 0 to 3, n1 represents an integer of 1 or greater, provided that, n1≤q1×2+4 is satisfied, and a benzene ring in Formula (a0-1) may have a substituent other than Ra⁰²¹.

3. The resist composition according to claim 1, wherein the constitutional unit (a0) is represented by General Formula (a0-2), 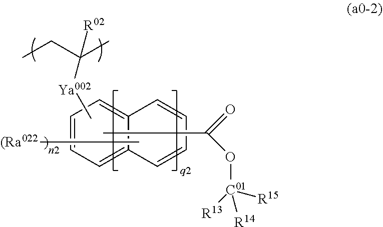
wherein R⁰² represents an alkyl group having 1 to 5 carbon atoms, a halogenated alkyl group having 1 to 5 carbon atoms, or a hydrogen atom, Ya⁰⁰² represents a divalent linking group or a single bond, C⁰² represents a secondary carbon atom or a tertiary carbon atom, any of α-positions of C⁰² represents a carbon atom constituting a carbon-carbon unsaturated bond with a β-position carbon atom or a carbon atom constituting an aromatic ring, R¹³ represents an aromatic group which may have a substituent, a chain unsaturated hydrocarbon group which may have a substituent, or a hydrogen atom, R¹⁴ and R¹⁵ each independently represent a chain hydrocarbon group which may have a substituent or a hydrogen atom, or a cyclic group which may have a substituent is formed by bonding R¹⁴ and R¹⁵ to each other, Ra⁰²² represents an iodine atom or a bromine atom, q2 represents an integer of 0 to 3, n2 represents an integer of 1 or greater, provided that, n2≤q2×2+4 is satisfied, and the benzene ring in Formula (a0-2) may have a substituent other than Ra⁰²².

4. The resist composition according to claim 1, wherein a proportion of the constitutional unit (a0) in the resin component (A1) is 20 mol % or greater and 80 mol % or less with respect 100 mol % of all constitutional units constituting the resin component (A1).

5. The resist composition according to claim 1, wherein the resin component (A1) further has a constitutional unit (a10) represented by General Formula (a10-1), 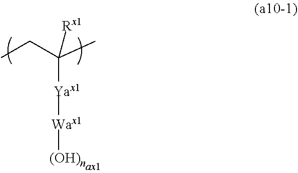
wherein R^{x1} represents an alkyl group having 1 to 5 carbon atoms, a halogenated alkyl group having 1 to 5 carbon atoms, or a hydrogen atom, Ya^{x1} represents a divalent linking group or a single bond,
Wa^{x1} represents an aromatic hydrocarbon group which may have a substituent, and n^{ax1} represents an integer of 1 or greater.

6. A resist pattern formation method comprising:
forming a resist film on a support using the resist composition according to claim 1;
exposing the resist film to light; and
developing the resist film exposed to light to form a resist pattern.

7. The resist pattern formation method according to claim 6, wherein the resist film is exposed with an extreme ultraviolet ray or an electron beam.

8. A compound represented by General Formula (a0-m0), 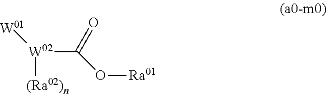
wherein W⁰¹ represents a polymerizable group-containing group, W⁰² represents an aromatic hydrocarbon group which may have a substituent, Ra⁰¹ represents is an acid dissociable group and represents a group having an alicyclic group, which has no carbon-carbon unsaturated bond, a group having a carbon-carbon unsaturated bond, or a group having an aromatic group, where the alicyclic group and the aromatic group may each have a substituent, Ra⁰² represents an iodine atom or a bromine atom, and n represents an integer of 1 or greater.

9. The compound according to claim 8, wherein the compound is represented by General Formula (a0-m1), 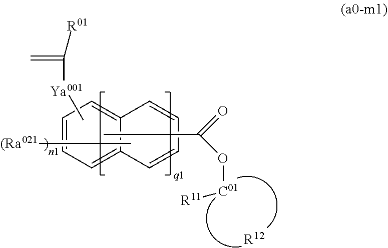
wherein R⁰¹ represents an alkyl group having 1 to 5 carbon atoms, a halogenated alkyl group having 1 to 5 carbon atoms, or a hydrogen atom, Ya⁰⁰¹ represents a divalent linking group or a single bond, C⁰¹ represents a tertiary carbon atom, R¹¹ represents a chain saturated hydrocarbon group which may have a substituent, R¹² represents a group forming an alicyclic group with C⁰¹, which has no carbon-carbon unsaturated bond, the alicyclic group here may have a substituent, Ra⁰²¹ represents an iodine atom or a bromine atom, q1 represents an integer of 0 to 3, n1 represents an integer of 1 or greater, provided that, n1≤q1×2+4 is satisfied, and the benzene ring in Formula (a0-m1) may have a substituent other than Ra⁰²¹.

10. The compound according to claim 8, wherein the compound is represented by General Formula (a0-m2), 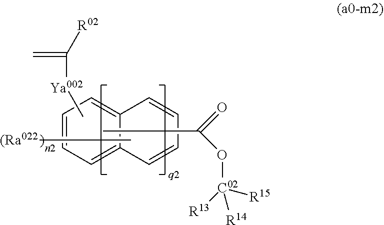
wherein R⁰ represents an alkyl group having 1 to 5 carbon atoms, a halogenated alkyl group having 1 to 5 carbon atoms, or a hydrogen atom, Ya⁰⁰² represents a divalent linking group or a single bond, C⁰² represents a secondary carbon atom or a tertiary carbon atom, any of α-positions of C⁰² represents a carbon atom constituting a carbon-carbon unsaturated bond with a β-position carbon atom or a carbon atom constituting an aromatic ring, R¹³ represents an aromatic group which may have a substituent, a chain-like unsaturated hydrocarbon group which may have a substituent, or a hydrogen atom, R¹⁴ and R¹⁵ each independently represent a chain-like hydrocarbon group which may have a substituent or a hydrogen atom, or a cyclic group which may have a substituent is formed by bonding R¹⁴ and R¹⁵ to each other, Ra⁰²² represents an iodine atom or a bromine atom, q2 represents an integer of 0 to 3, n2 represents an integer of 1 or greater, provided that, n2≤q2×2+4 is satisfied, and the benzene ring in Formula (a0-m2) may have a substituent other than Ra⁰²².
